(19) United States
(12) Patent Application Publication
(10) Pub. No.: US 2023/0015478 A1
(43) Pub. Date: Jan. 19, 2023

(54) GLP-1 RECEPTOR AGONISTS HAVING IMPROVED PHARMACOLOGICAL AND DRUG DELIVERY PROPERTIES

(71) Applicant: Resolute Bio, Inc., Beverly, MA (US)

(72) Inventors: Tomi K. Sawyer, Southborough, MA (US); Joseph Audie, Cheshire, CT (US); David J. Diller, East Windsor, NJ (US); Valentin Gribkoff, Wallingford, CT (US); Bradley L. Pentelute, Revere, MA (US); Solimar G. Santiago, Medford, MA (US); Jonathon R. Sawyer, Beverly, MA (US); Allison A. Shrier, Andover, MA (US); Jon T. Swanson, Wentzville, MO (US); Dinara S. Gunasekera, Cambridge, MA (US)

(21) Appl. No.: 17/839,981

(22) Filed: Jun. 14, 2022

Related U.S. Application Data
(60) Provisional application No. 63/210,321, filed on Jun. 14, 2021.

(51) Int. Cl. C07K 14/605

(52) U.S. Cl. CPC C07K 14/605; A61K 38/00

(57) ABSTRACT

Disclosed are polypeptides, compositions, and methods useful for activating a glucagon-like peptide-1 (GLP-1) receptor and treating or preventing diseases or disorders at least partially mediated by glucagon-like peptide 1 (GLP-1).

### RELATED APPLICATION

This application claims the benefit of priority to U.S. Provisional Patent Application No. 63/210,321, filed Jun. 14, 2021.

### BACKGROUND

Glucagon-like peptide-1 (GLP-1) is an important brain-gut peptide hormone having well-known physiological function in glucose metabolism, gastrointestinal secretion and metabolism, and furthermore therapeutic applications to diabetes, obesity and related metabolic disorders as well as emerging relationships to neurodegenerative diseases (1-16). Historically, GLP-1 was identified as an incretin hormone and shown to enhance meal-induced insulin secretion. It is a product of the glucagon gene encoding proglucagon. Human GLP-1 is a 30 amino acid peptide originating from preproglucagon, which is biosynthesized in the gastrointestinal tract (e.g., A-cells of the pancreas and L-cells in the distal ileum) and in the brain. Processing of preproglucagon to yield GLP-1(7-36)—NH2 and GLP-2 occurs mainly in the L-cells. GLP-1 is normally secreted in response to food intake, in particular carbohydrates and lipids stimulate GLP-1 secretion.

GLP-1 has been identified as a very potent and efficacious stimulator for insulin release. GLP-1 lowers plasma glucagon concentrations, slows gastric emptying, stimulates insulin biosynthesis and enhances insulin sensitivity. Furthermore, GLP-1 enhances the ability of the beta-cells to sense and respond to glucose in subjects with impaired glucose tolerance. The insulinotropic effect of GLP-1 in humans increases the rate of glucose metabolism partly due to increased insulin levels and partly due to enhanced insulin sensitivity. GLP-1 exerts non-insulinotropic actions, such as controlling pancreatic R cell proliferation and survival, bone metabolism, controlling food intake and satiety, enhancing proliferation of neuronal progenitors and protection against neuronal apoptosis, reducing cardiac contractility and improving cardiac performance following cardiac injuries. Collectively, such known pharmacological properties of GLP-1 make it a highly desirable therapeutic agent for the treatment of type-II diabetes, obesity and related metabolic disorders and their complications, including nonalcoholic steatohepatitis, with potential roles in neurodegenerative diseases and cardioprevention.

### SUMMARY

One aspect of the invention provides polypeptides, compositions, and methods useful for activating a glucagon-like peptide-1 (GLP-1) receptor.

Accordingly, provided herein is a polypeptide represented by the following sequence (I):

R_{XN}—Xₐₐ¹—Xₐₐ²—Xₐₐ³—Xₐₐ⁴—Xₐₐ⁵—Xₐₐ⁶—Xₐₐ⁷—Xₐₐ⁸—Xₐₐ⁹—Xₐₐ¹⁰—Xₐₐ¹¹—R_{yc} (I)

wherein

R_{XN} is the N-terminal group of Xₐₐ¹ selected from H (i.e., des-amino) and —N(Rx)₂, wherein Rx, independently for each occurrence, is H or an optionally substituted alkyl, arylalkyl, heteroarylalkyl, formyl, acetyl, alkanoyl, —C(O)-alkyloxy, —C(O)-aryloxy, —C(O)-arylalkyloxy, —C(O)-heterocyclyloxy, —C(O)-heteroarylalkyloxy, —C(O)NH-alkyl, —C(O)NH-aryl, —C(O)NH— aralkyl, —SO₂-heterocyclyl, —SO₂-alkyl, —SO₂-aryl, —SO₂-arylalkyl, —SO₂-heteroarylalkyl, —SO₂-heteroaryl, or ureido; or one occurrence of Rx is hydrogen and the other occurrence is an amino acid residue Xₐₐ⁰;

Xₐₐ⁰ is an optionally substituted amino acid residue selected from Gly, Pro, Arg, Glu, His, Phe and Trp;

Xₐₐ^{l} is an optionally substituted amino acid residue comprising an amino acid side chain that comprises an alkyl, aryl or heteroaryl;

Xₐₐ² is an optionally substituted amino acid residue selected from Gly, Aib, Ala, D-Ala, N-methyl-Ala, N-methyl-D-Ala, Pro, α-methyl-Pro, Val, D-Val, and D-His;

Xₐₐ³ is an optionally substituted amino acid residue comprising an amino acid side chain that comprises a carboxyl or sulfonic acid group;

Xₐₐ⁴ is an amino acid residue selected from Gly, Ala, Aib, and β-Ala;

Xₐₐ⁵ is an optionally substituted amino acid selected from Thr, Ser, Ala, Aib, Val, α-MeSer, α-MeThr, and α-MeVal;

Xₐₐ⁶ is an optionally substituted amino acid residue that is disubstituted at the α carbon, provided that one of the substituents is an optionally substituted aryl or heteroaryl;

Xₐₐ⁷ is an optionally substituted amino acid residue comprising an amino acid side chain that comprises a hydroxyl;

Xₐₐ⁸ is an optionally substituted amino acid residue selected from Ser, His, and Asn;

Xₐₐ⁹ is an optionally substituted amino acid residue comprising an amino acid side chain that comprises a carboxyl or sulfonic acid group;

Xₐₐ¹⁰ is an optionally substituted amino acid residue comprising an amino acid side chain that comprises a sulfide and/or an optionally substituted aryl or heteroaryl;

Xₐₐ¹¹ is an optionally substituted amino acid residue comprising an amino acid side chain that comprises a sulfide and/or an optionally substituted aryl or heteroaryl; and

R_{YC} is the C-terminal group of Xₐₐ¹¹ having the structure —C(O)N(R_{Y})₂, wherein R_{Y}, independently for each occurrence, is hydrogen or a PK modifier group.

Another aspect of the invention relates to methods of treating or preventing a disease or disorder at least partially mediated by glucagon-like peptide 1 in a subject in need thereof comprising administering to the subject an effective amount of a polypeptide of Formula (I).

A further aspect of the invention relates to a method of treating or preventing diabetes in a subject in need thereof comprising administering to the subject an effective amount of a polypeptide of sequence (I).

A further aspect of the invention relates to a method of treating or preventing a neurodegenerative disease in a subject in need thereof comprising administering to the subject an effective amount of a polypeptide of sequence (I).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWING

FIG. 1. Correlation between descriptor-based pEC50 calculations and experimental pEC50 results for 28 GLP-1 peptide analogs based on 5nx2-based model structures.

### DETAILED DESCRIPTION

Disclosed herein are polypeptide GLP-1 receptor agonists, which exhibit superior pharmacological properties relative to the native peptide, GLP-1, with respect to GLP-1 receptor activation, metabolic stability and pharmacokinetics (by parenteral or oral drug delivery). Accordingly, the disclosed polypeptides are useful for the treating or preventing GLP-1 related metabolic disorders, including type II diabetes and obesity, and neurodegenerative disease.

The polypeptides disclosed herein modulate the GLP-1 receptor, e.g. as an agonists or partial agonists of the GLP-1 receptor. The peptides disclosed herein exhibit similar or superior in vivo pharmacological and pharmacokinetic properties relative to GLP-1, thus making them ideal therapeutic candidates for subcutaneous, oral, pulmonary, nasal, buccal routes of drug delivery (including the use of sustained release formulations and/or excipients to enhance permeability for uptake into systemic circulation as dependent upon the exact route of drug delivery). In particular, the polypeptides disclosed herein exhibit superior postprandial plasma glucose lowering and concomitant increase in plasma insulin levels like other agonists of GLP-1 receptor. Agonists of GLP-1 receptor have shown clinical benefit in diabetes and its micro and macrovascular complications as well as obesity and related metabolic disorders and are undergoing evaluation in neurodegenerative diseases, nonalcoholic steatohepatosis (NASH), metabolic disorders in the setting of HIV and its treatment, polycystic ovary syndrome (PCOS), and cardioprevention. Accordingly, the disclosed polypeptides are effective in treating or preventing complications in type II diabetes and related metabolic disorders including NASH and obesity as well as neurodegenerative disease.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, compounds of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon—carbon double bond may be in an E (substituents are on opposite sides of the carbon—carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C—or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl goups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example, from 2 to 12 carbon atoms, which contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to a cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

“Ureido” refers to an optionally substituted urea moiety, e.g., —NHC(O)NH₂ or —NHC(O)NHR, wherein R is alkyl, aryl, aralkyl, heterocyclyl, heteroaryl, or heteroarylalkyl.

A “PK modifier group” refers to a group which alters, e.g. improves, the pharmacokinetic (PK) profile of the polypeptide to which it is attached. A PK modifier group may exploit known binders to human serum albumin (HSA). This interaction with albumin may result in in reduced in vivo clearance. Therefore, an example of a PK modifier group is a serum albumin binding group.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

The term “diabetes and related diseases or related conditions” refers, without limitation, to Type II diabetes, Type I diabetes, impaired glucose tolerance, obesity, hyperglycemia, Syndrome X, dysmetabolic syndrome, diabetic complications, and hyperinsulinemia.

For purposes of this invention, the chemical elements are identified in accordance with the Periodic Table of the Elements, CAS version, Handbook of Chemistry and Physics, 67th Ed., 1986-87, inside cover.

Compounds of the Invention One aspect of the invention relates to a polypeptide represented by the following sequence (I):

R_{XN}—Xₐₐ¹—Xₐₐ²—Xₐₐ³—Xₐₐ⁴—Xₐₐ⁵—Xₐₐ⁶—Xₐₐ⁷—Xₐₐ⁸—Xₐₐ⁹—Xₐₐ¹⁰—Xₐₐ¹¹—R_{yc} (I)

wherein

R_{XN} is the N-terminal group of Xₐₐ¹ selected from H (i.e., des-amino) and —N(Rx)₂, wherein Rx, independently for each occurrence, is H or an optionally substituted alkyl, arylalkyl, heteroarylalkyl, formyl, acetyl, alkanoyl, —C(O)-alkyloxy, —C(O)-aryloxy, —C(O)-aralkyloxy, —C(O)-heterocyclyloxy, —C(O)-heteroarylalkyloxy, —C(O)NH-alkyl, —C(O)NH-aryl, —C(O)NH— arylalkyl, —SO₂-heterocyclyl, —SO₂-alkyl, —SO₂-aryl, —SO₂-arylalkyl, —SO₂-heteroarylalkyl, —SO₂-heteroaryl, or ureido; or one occurrence of Rx is hydrogen and the other occurrence is an amino acid residue Xₐₐ⁰;

Xₐₐ⁰ is an optionally substituted amino acid residue selected from Gly, Pro, Arg, Glu, His, Phe and Trp;

Xₐₐ^{l} is an optionally substituted amino acid residue comprising an amino acid side chain that comprises an alkyl, aryl or heteroaryl;

Xₐₐ² is an optionally substituted amino acid residue selected from Gly, Aib, Ala, D-Ala, N-methyl-Ala, N-methyl-D-Ala, Pro, α-methyl-Pro, Val, D-Val, and D-His;

Xₐₐ³ is an optionally substituted amino acid residue comprising an amino acid side chain that comprises a carboxyl or sulfonic acid group;

Xₐₐ⁴ is an amino acid residue selected from Gly, Ala, Aib, and β-Ala;

Xₐₐ⁵ is an optionally substituted amino acid selected from Thr, Ser, Ala, Aib, Val, α-MeSer, α-MeThr, and α-MeVal;

Xₐₐ⁶ is an optionally substituted amino acid residue that is disubstituted at the α carbon, provided that one of the substituents is an optionally substituted aryl or heteroaryl;

Xₐₐ⁷ is an optionally substituted amino acid residue comprising an amino acid side chain that comprises a hydroxyl;

Xₐₐ⁸ is an optionally substituted amino acid residue selected from Ser, His, and Asn;

Xₐₐ⁹ is an optionally substituted amino acid residue comprising an amino acid side chain that comprises a carboxyl or sulfonic acid group;

Xₐₐ¹⁰ is an optionally substituted amino acid residue comprising an amino acid side chain that comprises a sulfide and/or an optionally substituted aryl or heteroaryl; Xₐₐ¹¹ is an optionally substituted amino acid residue comprising an amino acid side chain that comprises a sulfide and/or an optionally substituted aryl or heteroaryl; and

R_{YC} is the C-terminal group of Xₐₐ¹¹ having the structure —C(O)N(R_{Y})₂, wherein R_{Y}, independently for each occurrence, is hydrogen or a PK modifier group.

In certain embodiments, R_{YC} is the C-terminal group of Xₐₐ¹¹ having the structure —C(O)N(R_{Y})₂, wherein R_{Y}, independently for each occurrence, is hydrogen or a serum albumin binding group.

In certain embodiments, Xₐₐ^{l} is an optionally substituted amino acid residue comprising an amino acid side chain that comprises a (C₁-C₄) alkyl, imidazole, or phenyl.

In certain embodiments, Xₐₐ^{l} is an optionally substituted amino acid residue selected from, Leu, His, and Tyr.

In certain embodiments, the amino acid residue, when substituted, is substituted with at least one halo, hydroxyl, or alkyl.

In certain embodiments, Xₐₐ² is an unsubstituted amino acid residue selected from Gly, Aib, Ala, D-Ala, N-methyl-Ala, N-methyl-D-Ala, Pro, α-methyl-Pro, Val, and D-Val.

In certain embodiments, Xₐₐ² is a substituted amino acid residue selected from Gly, Aib, Ala, D-Ala, N-methyl-Ala, N-methyl-D-Ala, Pro, α-methyl-Pro, Val, and D-Val.

In certain embodiments, the amino acid residue is selected from Aib, Pro, α-methyl-Pro, and Val.

In certain embodiments, the amino acid residue is substituted with at least one halo or alkyl.

In certain embodiments, Xₐₐ³ is an optionally substituted amino acid residue comprising an amino acid side chain that comprises a carboxyl.

In certain embodiments, Xₐₐ³ is an amino acid residue selected from Asp and Glu.

In certain embodiments, Xₐₐ³ is an optionally substituted amino acid residue comprising an amino acid side chain that comprises a sulfonic acid group.

In certain embodiments, Xₐₐ³ is an amino acid residue selected from cysteic acid.

In certain embodiments, the amino acid residue, when substituted, is substituted with at least one halo or alkyl.

In certain embodiments, Xₐₐ⁴ is an amino acid residue selected from Gly and Ala.

In certain embodiments, Xₐₐ⁵ is an unsubstituted amino acid residue selected from Thr, Ser, Ala, Aib, Val, α-MeSer, α-MeThr, and α-MeVal.

In certain embodiments, Xₐₐ⁵ is a substituted amino acid residue selected from Thr, Ser, Ala, Aib, Val, α-MeSer, α-MeThr, and α-MeVal.

In certain embodiments, Xₐₐ⁵ is unsubstituted or substituted Thr.

In certain embodiments, Xₐₐ⁵ is substituted with at least one halo or alkyl.

In certain embodiments, Xₐₐ⁶ is an optionally substituted amino acid residue represented by:

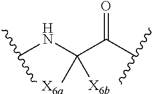

wherein X₆a is alkyl; and X_{6b} is substituted arylalkyl.

In certain embodiments, the arylalkyl is substituted with at least one halo.

In certain embodiments, X₆ₐ is methyl; and X_{6b} is benzyl, 2-fluorobenzyl, or 2,4-difluorobenzyl.

In certain embodiments, Xₐₐ⁶ is an optionally substituted amino acid residue selected from α-MePhe, α-MePhe(₂-F), and α-MePhe(2,6-DiF) In certain embodiments, Xₐₐ⁷ is an optionally substituted amino acid residue selected from Thr, α-MeThr, Ser, and α-MeSer.

In certain embodiments, the amino acid residue, when substituted, is substituted with at least one halo or alkyl.

In certain embodiments, Xₐₐ⁸ is an unsubstituted amino acid residue selected from Ser, His, and Asn.

In certain embodiments, Xₐₐ⁸ is a substituted amino acid residue selected from Ser, His, and Asn.

In certain embodiments, Xₐₐ⁸ is unsubstituted or substituted Ser.

In certain embodiments, Xₐₐ⁸, when substituted, is substituted with at least one halo or alkyl.

In certain embodiments, Xₐₐ⁹ is an optionally substituted amino acid residue comprising an amino acid side chain that comprises a carboxyl.

In certain embodiments, Xₐₐ⁹ is an amino acid residue selected from Asp and Glu.

In certain embodiments, Xₐₐ⁹ is an optionally substituted amino acid residue comprising an amino acid side chain that comprises a sulfonic acid group.

In certain embodiments, Xₐₐ⁹ is optionally substituted cysteic acid.

In certain embodiments, the amino acid residue, when substituted, is substituted with at least one halo or alkyl.

In certain embodiments, Xₐₐ¹⁰ is an amino acid residue comprising an amino acid side chain that comprises a substituted aryl.

In certain embodiments, Xₐₐ¹⁰ is further substituted at the α-carbon. In certain embodiments, Xₐₐ¹⁰ is further substituted with an alkyl at the α-carbon. In other embodiments, Xₐₐ¹⁰ is further substituted with a methyl at the α-carbon.

In certain embodiments, Xₐₐ¹⁰ is represented by:

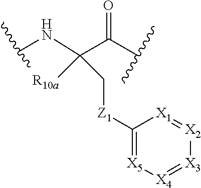

wherein

X₁ is N or CR_{10b};

X₂ is N or CR_{10c};

X₃ is N or CR_{10d};

X₄ is N or CR₁₀ₑ;

X₅ is N or CR_{10f};

Z₁ is absent or present, and when present is S or SO₂;

R₁₀ₐ is H or alkyl; and

R_{10b}, R_{10c}, R_{10d}, R₁₀ₑ, and R_{10f} are independently selected from H, halogen, and alkyl.

In certain embodiments, Xₐₐ^{l} is represented by:

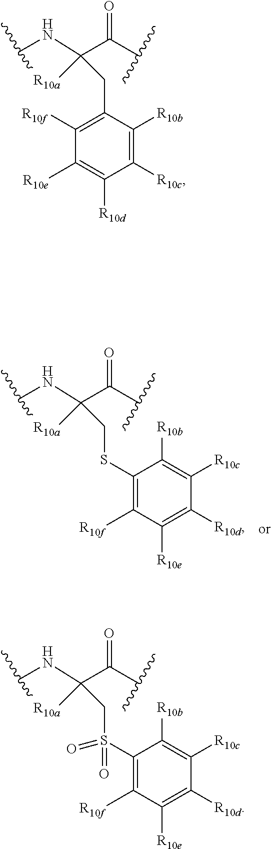

In certain embodiments, Xₐₐ¹⁰ is represented by:

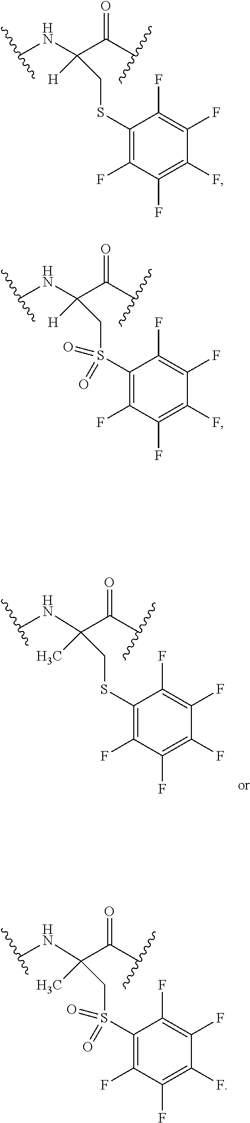 [SMILES: *NC(CSc1c(F)c(F)c(F)c(F)c1F)C(*)=O ; *NC(CS(=O)(=O)c1c(F)c(F)c(F)c(F)c1F)C(*)=O ; *NC(C)(CSc1c(F)c(F)c(F)c(F)c1F)C(*)=O ; *NC(C)(CS(=O)(=O)c1c(F)c(F)c(F)c(F)c1F)C(*)=O]

In certain embodiments, Xₐₐ¹⁰ is represented by:

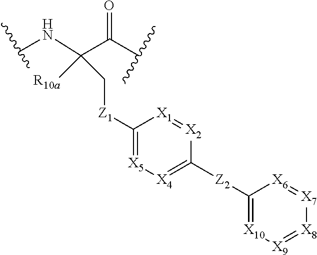

wherein

X₁ is N or CR_{10b};

X₂ is N or CR_{10c};

X₄ is N or CR₁₀ₑ;

X₅ is N or CR_{10f};

X₆ is N or CR_{10g};

X₇ is N or CR₁₀ₕ;

X₈ is N or CR₁₀ᵢ;

X₉ is N or CR₁₀ⱼ;

X₁₀ is N or R₁₀ₖ;

Z₁ is absent or present, and when present is S or SO₂;

Z₂ is absent or present, and when present is S or SO₂;

R₁₀ₐ is selected from H and alkyl; and

R_{10b}, R_{10c}, R₁₀ₑ, R_{10f}, R_{10g}, R₁₀ₕ, R₁₀ᵢ, R₁₀ⱼ, and R₁₀ₖ are independently selected from H, halogen, and alkyl.

In certain embodiments, Xₐₐ¹⁰ is represented by:

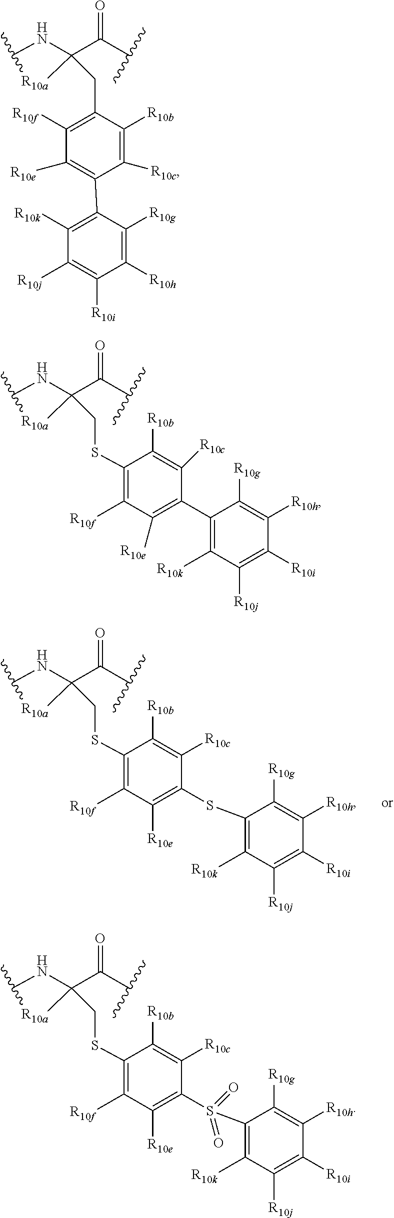

In certain embodiments, Xₐₐ¹⁰ is represented by:

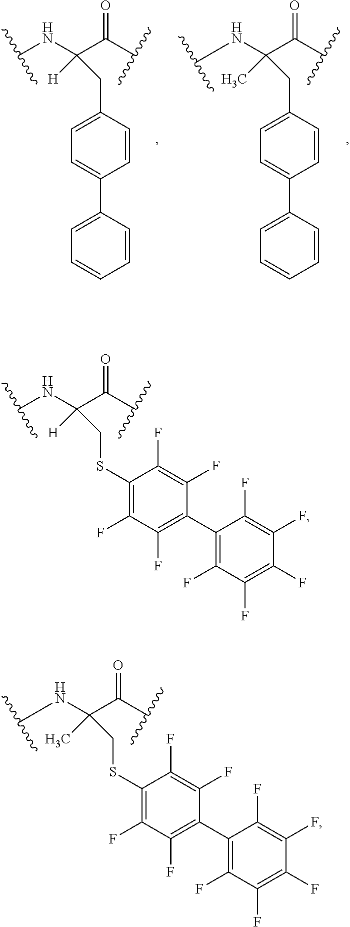 [SMILES: *NC(Cc1ccc(-c2ccccc2)cc1)C(*)=O ; *NC(C)(Cc1ccc(-c2ccccc2)cc1)C(*)=O ; *NC(CSc1c(F)c(F)c(-c2c(F)c(F)c(F)c(F)c2F)c(F)c1F)C(*)=O ; *NC(C)(CSc1c(F)c(F)c(-c2c(F)c(F)c(F)c(F)c2F)c(F)c1F)C(*)=O]

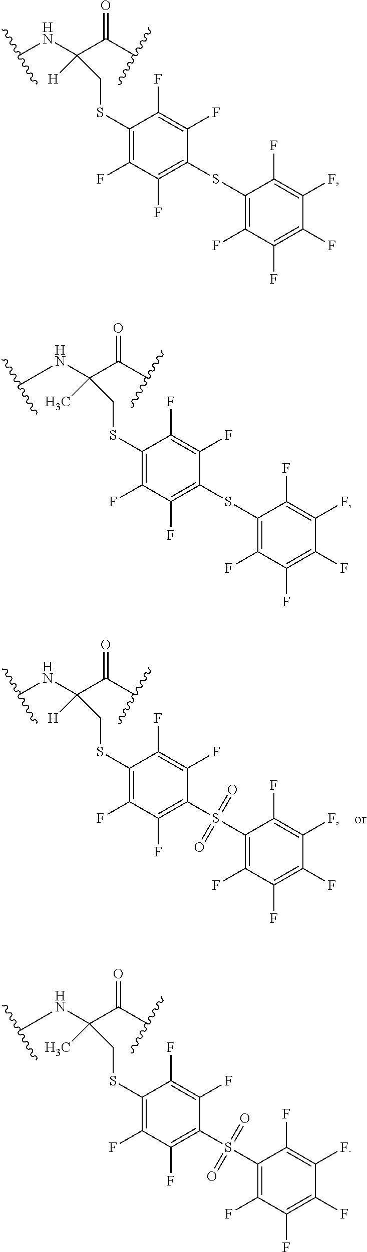 [SMILES: *NC(CSc1c(F)c(F)c(Sc2c(F)c(F)c(F)c(F)c2F)c(F)c1F)C(*)=O ; *NC(C)(CSc1c(F)c(F)c(Sc2c(F)c(F)c(F)c(F)c2F)c(F)c1F)C(*)=O ; *NC(CSc1c(F)c(F)c(S(=O)(=O)c2c(F)c(F)c(F)c(F)c2F)c(F)c1F)C(*)=O ; *NC(C)(CSc1c(F)c(F)c(S(=O)(=O)c2c(F)c(F)c(F)c(F)c2F)c(F)c1F)C(*)=O]

In certain embodiments, Xₐₐ¹⁰ is represented by:

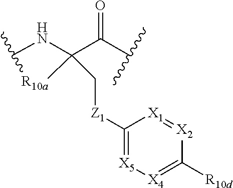

wherein

X₁ is N or CR_{10b};

X₂ is N or CR_{10c};

X₄ is N or CR₁₀ₑ;

X₅ is N or CR_{10f};

Z₁ is absent or present, and when present is selected from CH₂, S, O, NH, SO₂, SO₂—NH, NH—SO₂, NHC(O), and C(O)NH;

R₁₀ₐ is selected from H and alkyl;

R_{10d} is —OH or -L₁-L₂-L₃-R_{10d}′;

R₁₀ₐ′ is —NH₂, (C₁-C₂₀ alkyl)-CO₂H or optionally substituted (C₁-C₆ alkyl)-aryl;

L₁ is absent or present and when present is a linker;

L₂ is a linker which comprises an ether moiety;

L₃ is absent or present and when present is a linker that comprises an amino acid moiety; and

R_{10b}, R_{10c}, R_{10d}, R₁₀ₑ and R_{10f} are independently selected from H, halogen, and alkyl.

In certain embodiments, R_{10d} is-L₁-L₂-L₃-R₁₀ₐ′.

In certain embodiments, Xₐₐ¹⁰ is represented by:

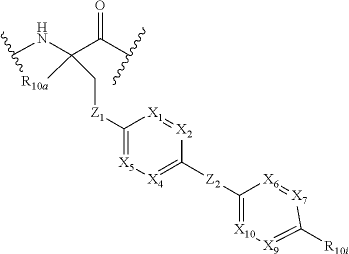

wherein

X₁ is N or CR_{10b};

X₂ is N or CR_{10c};

X₄ is N or CR₁₀ₑ;

X₅ is N or CR_{10f};

X₆ is N or CR_{10g};

X₇ is N or CR₁₀ₕ;

X₉ is N or CR₁₀ⱼ;

X₁₀ is N or CR₁₀ₖ;

Z₁ is absent or present, and when present is selected from CH₂, NH, S, So₂, O, SO₂—NH, NH—SO₂, NHC(O), and C(O)NH;

Z₂ is absent or present, and when present is selected from NH, S, SO₂, O, SO₂—NH, NH—SO₂, NHC(O), and C(O)NH;

R₁₀ₐ is selected from H and alkyl;

R₁₀ᵢ is —L₁-L₂-L₃-R₁₀ᵢ′;

R₁₀ᵢ′ is —NH₂, (C₁-C₂₀ alkyl)-CO₂H or optionally substituted (C₁-C₆ alkyl)-aryl;

L₁ is absent or present and when present is a linker;

L₂ is a linker which comprises an ether moiety;

L₃ is absent or present and when present is a linker that comprises an amino acid moiety; and

R_{10b}, R_{10c}, R₁₀ₑ, R_{10f}, R_{10g}, R₁₀ₕ, R₁₀ⱼ, and R₁₀ₖ are independently selected from H, halogen, and alkyl.

In certain embodiments, Xₐₐ¹⁰ is represented by:

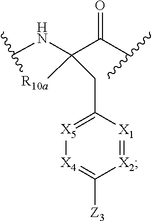

wherein

X₁ is N or CR_{10b};

X₂ is N or CR_{10c};

X₄ is N or CR₁₀ₑ;

X₅ is N or CR_{10f};

R₁₀ₐ is selected from H and alkyl;

R_{10b}, R_{10c}, R₁₀ₑ, and R_{10f} are independently selected from H, halogen, and alkyl; and

Z₃ is a substituted heteroaryl.

In certain embodiments, Z₃ is a substituted 5-membered heteroaryl.

In certain embodiments, the substituted 5-membered heteroaryl is a substituted triazolyl.

In certain embodiments, the substituted heteroaryl is substituted with a —(C₁₋₂₀ alkyl)-CO₂H.

In certain embodiments, the substituted heteroaryl is substituted with a —(C₁₀₋₂₀ alkyl)-CO₂H.

In certain embodiments, the substituted heteroaryl is substituted with a —(C₁₅ alkyl)-CO₂H.

In certain embodiments, Xₐₐ¹⁰ is represented by:

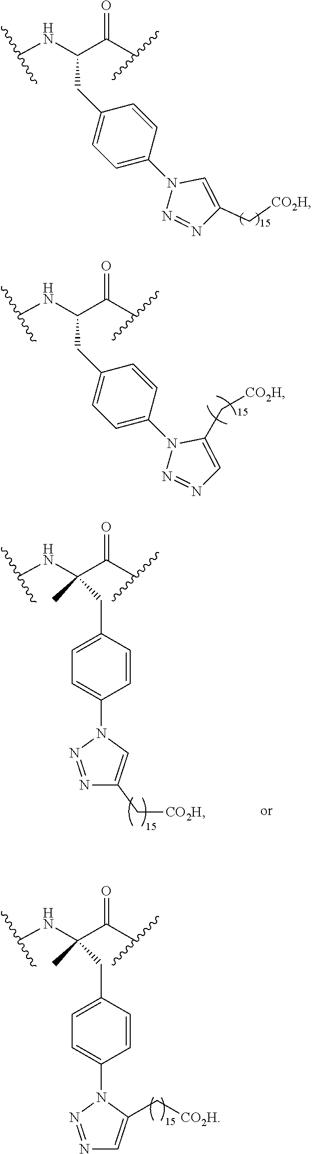

In certain embodiments, Xₐₐ¹¹ is an amino acid residue comprising an amino acid side chain that comprises a substituted aryl.

In certain embodiments, Xₐₐ¹¹ is further substituted at the α-carbon. In certain embodiments, Xₐₐ¹¹ is further substituted with an alkyl at the α-carbon. In other embodiments, Xₐₐ¹¹ is further substituted with a methyl at the α-carbon.

In certain embodiments, Xₐₐ¹¹ is represented by:

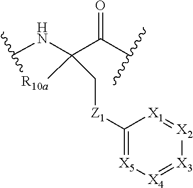

wherein X₁ is N or CR_{10b}; X₂ is N or CR_{10c}; X₃ is N or CR_{10d}; X₄ is N or CR₁₀ₑ;

X₅ is N or CR_{10f};

Z₁ is absent or present, and when present is S or SO₂;

R₁₀ₐ is selected from H and alkyl; and

R_{10b}, R_{10c}, R_{10d}, R₁₀ₑ, and R_{10f} are independently selected from H, halogen, and alkyl.

In certain embodiments, Xₐₐ¹¹ is represented by:

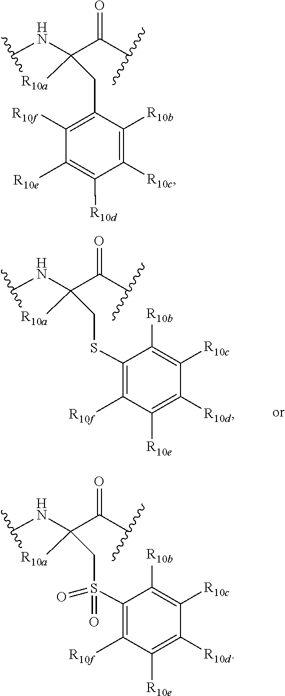

In certain embodiments, Xₐₐ¹¹ is represented by:

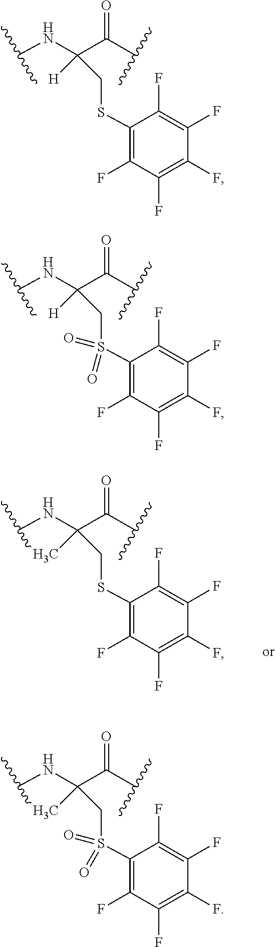 [SMILES: *NC(CSc1c(F)c(F)c(F)c(F)c1F)C(*)=O ; *NC(CS(=O)(=O)c1c(F)c(F)c(F)c(F)c1F)C(*)=O ; *NC(C)(CSc1c(F)c(F)c(F)c(F)c1F)C(*)=O ; "or" ; *NC(C)(CS(=O)(=O)c1c(F)c(F)c(F)c(F)c1F)C(*)=O]

In certain embodiments, in Xₐₐ¹¹ is represented by:

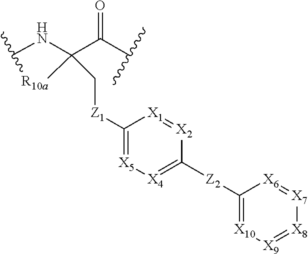

wherein

X₁ is N or CR_{10b};

X₂ is N or CR_{10c};

X₄ is N or CR₁₀ₑ;

X₅ is N or CR_{10f};

X₆ is N or CR_{10g};

X₇ is N or CR₁₀ₕ;

X₈ is N or CR₁₀ᵢ;

X₉ is N or CR₁₀ⱼ;

X₁₀ is N or CR₁₀ₖ;

Z₁ is absent or present, and when present is S or SO₂;

Z₂ is absent or present, and when present is S or SO₂;

R₁₀ₐ is selected from H and alkyl; and

R_{10b}, R_{10c}, R₁₀ₑ, R_{10f}, R_{10g}, R₁₀ₕ, R₁₀ᵢ, R₁₀ⱼ, and R₁₀ₖ are independently selected from H, halogen, and alkyl.

In certain embodiments, Xₐₐ¹¹ is represented by:

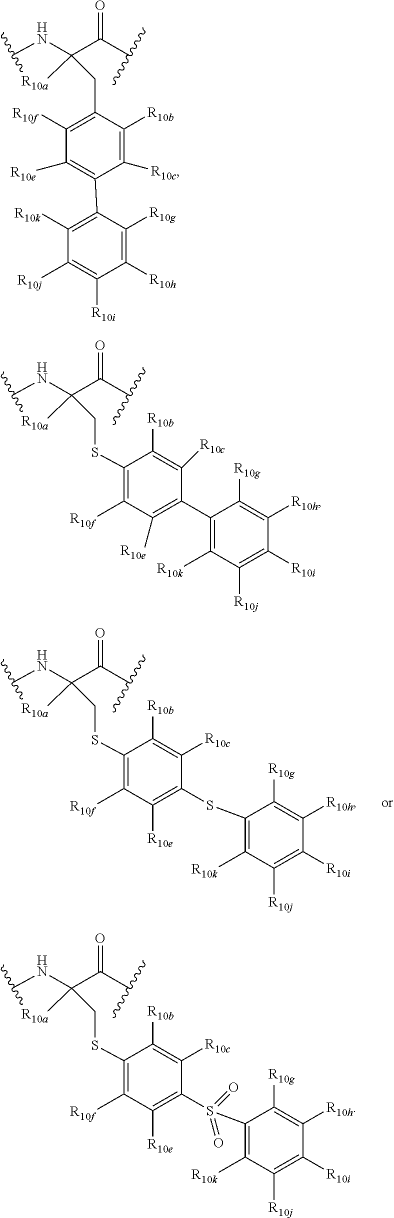

In certain embodiments, Xₐₐ^{l} is represented by:

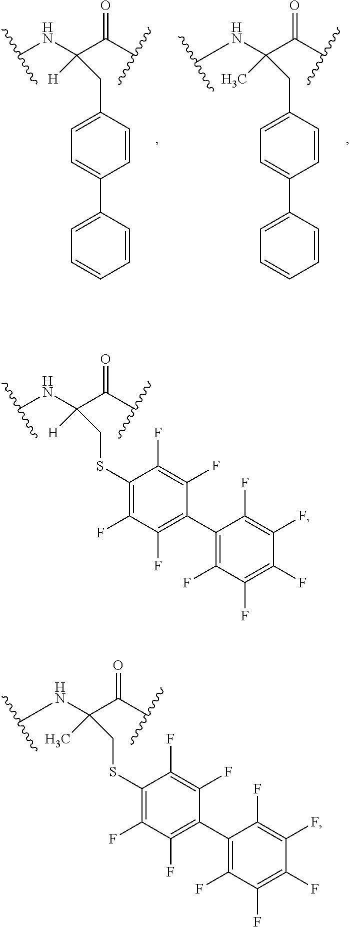 [SMILES: *NC(Cc1ccc(-c2ccccc2)cc1)C(*)=O ; *NC(C)(Cc1ccc(-c2ccccc2)cc1)C(*)=O ; *NC(CSc1c(F)c(F)c(-c2c(F)c(F)c(F)c(F)c2F)c(F)c1F)C(*)=O ; *NC(C)(CSc1c(F)c(F)c(-c2c(F)c(F)c(F)c(F)c2F)c(F)c1F)C(*)=O]

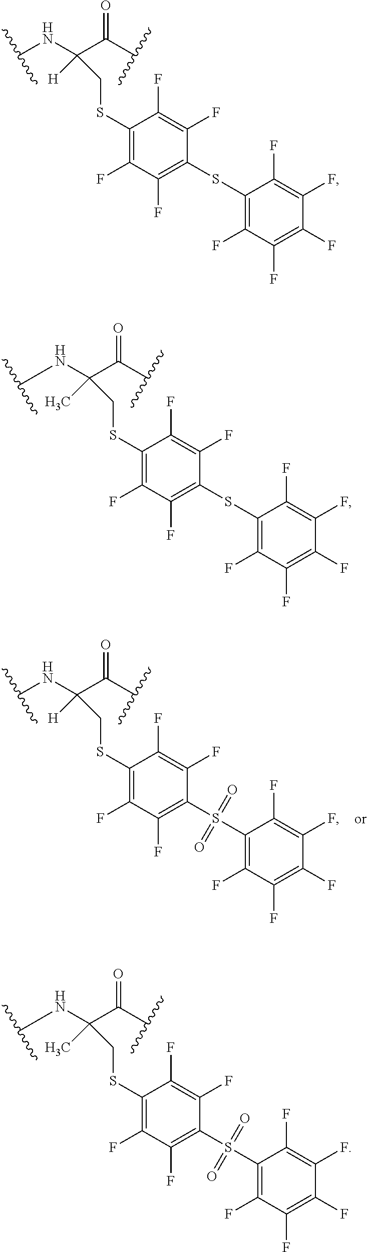 [SMILES: *NC(CSc1c(F)c(F)c(Sc2c(F)c(F)c(F)c(F)c2F)c(F)c1F)C(*)=O ; *NC(C)(CSc1c(F)c(F)c(Sc2c(F)c(F)c(F)c(F)c2F)c(F)c1F)C(*)=O ; *NC(CSc1c(F)c(F)c(S(=O)(=O)c2c(F)c(F)c(F)c(F)c2F)c(F)c1F)C(*)=O ; *NC(C)(CSc1c(F)c(F)c(S(=O)(=O)c2c(F)c(F)c(F)c(F)c2F)c(F)c1F)C(*)=O]

In certain embodiments, Xₐₐ¹¹ is represented by:

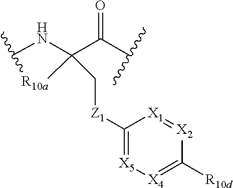

wherein

X₁ is N or CR_{10b};

X₂ is N or CR_{10c};

X₄ is N or CR₁₀ₑ;

X₅ is N or CR_{10f};

Z₁ is absent or present, and when present is selected from CH₂, S, O, NH, SO₂, SO₂—NH, NH—SO₂, NHC(O), and C(O)NH;

R₁₀ₐ is selected from H and alkyl;

R_{10d}′ is —OH or -L₁-L₂-L₃-R_{10d}′;

R₁₀ₐ′ is —NH₂, (C₁-C₂₀ alkyl)-CO₂H or optionally substituted (C₁-C₆ alkyl)-aryl;

L₁ is absent or present and when present is a linker;

L₂ is a linker which comprises an ether moiety;

L₃ is absent or present and when present is a linker that comprises an amino acid moiety; and

R_{10b}, R_{10c}, R_{10d}, R₁₀ₑ, and R_{10f} are independently selected from H, halogen, and alkyl.

In certain embodiments, Rio is —L₁-L₂-L₃-R₁₀ₐ′.

In certain embodiments, Xₐₐ¹¹ is represented by:

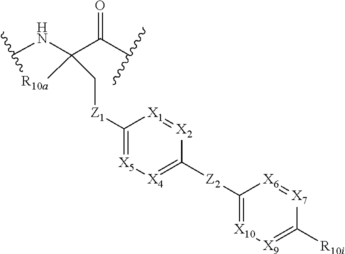

wherein

X₁ is N or CR_{10b};

X₂ is N or CR_{10c};

X₄ is N or CR₁₀ₑ;

X₅ is N or CR_{10f};

X₆ is N or CR_{10g};

X₇ is N or CR₁₀ₕ;

X₉ is N or CR₁₀ⱼ;

X₁₀ is N or CR₁₀ₖ;

Z₁ is absent or present, and when present is selected from CH₂, NH, S, SO₂, O, SO₂—NH, NH—SO₂, NHC(O), and C(O)NH;

Z₂ is absent or present, and when present is selected from NH, S, SO₂, O, SO₂—NH, NH—SO₂, NHC(O), and C(O)NH;

R₁₀ₐ is selected from H and alkyl;

R₁₀ᵢ is —OH or -L₁-L₂-L₃-R₁₀ᵢ′;

R₁₀ᵢ′ is —NH₂, (C₁-C₂₀ alkyl)-CO₂H or optionally substituted (C₁-C₆ alkyl)-aryl;

L₁ is absent or present and when present is a linker;

L₂ is a linker which comprises an ether moiety;

L₃ is absent or present and when present is a linker that comprises an amino acid moiety; and

R_{10b}, R_{10c}, R₁₀ₑ, R_{10f}, R_{10g}, R₁₀ₕ, R₁₀ⱼ, and R₁₀ₖ are independently selected from H, halogen, and alkyl.

In certain embodiments, R₁₀ᵢ is —L₁-L₂-L₃-R₁₀ᵢ′.

In certain embodiments, Xₐₐ¹¹ is represented by:

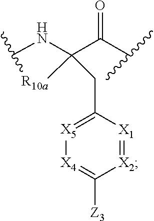

wherein

X₁ is N or CR_{10b};

X₂ is N or CR_{10c};

X₄ is N or CR₁₀ₑ;

X₅ is N or CR_{10f};

R₁₀ₐ is selected from H and alkyl;

R_{10b}, R_{10c}, R₁₀ₑ, and R_{10f} are independently selected from H, halogen, and alkyl; and

Z₃ is a substituted heteroaryl.

In certain embodiments, Z₃ is a substituted 5-membered heteroaryl.

In certain embodiments, the substituted 5-membered heteroaryl is a substituted triazolyl.

In certain embodiments, the substituted heteroaryl is substituted with a —(C₁₋₂₀ alkyl)-CO₂H.

In certain embodiments, the substituted heteroaryl is substituted with a —(C₁₀₋₂₀ alkyl)-CO₂H.

In certain embodiments, the substituted heteroaryl is substituted with a —(C₁₅ alkyl)-CO₂H.

In certain embodiments, Xₐₐ¹¹ is represented by:

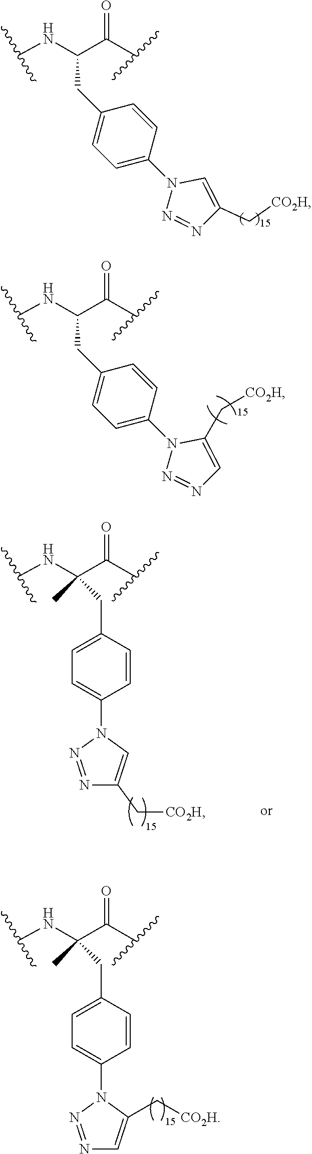

In certain embodiments, L₁, when present, is selected from

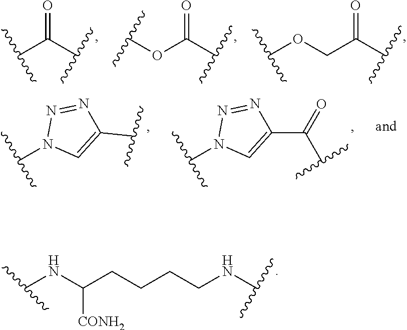

In certain embodiments, L₂ is

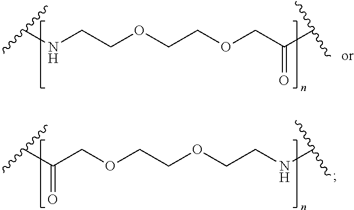

and each n is independently 1-6.

In certain embodiments, L₃, when present, is

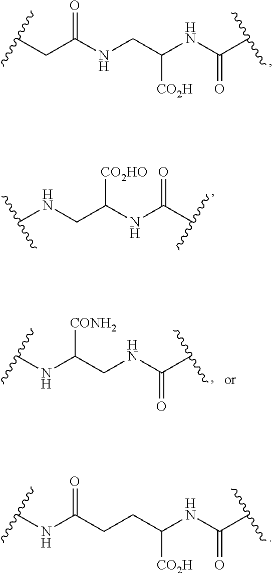

In certain embodiments, R_{10d}′ is (C₁-C₁₅ alkyl)-CO₂H.

In certain embodiments, R_{10d}′ is

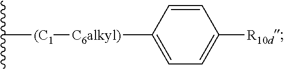

and R_{10d}″ is a halo.

In certain embodiments, R_{10d}″ is I.

In certain embodiments, R₁₀ᵢ″ is (C₁-C₁₅ alkyl)-CO₂H.

In certain embodiments, R₁₀ᵢ′ is

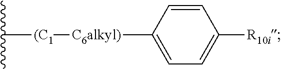

and R₁₀ᵢ″ is a halo.

In certain embodiments, R₁₀ᵢ″ is I.

In certain embodiments, R_{10d} is selected from

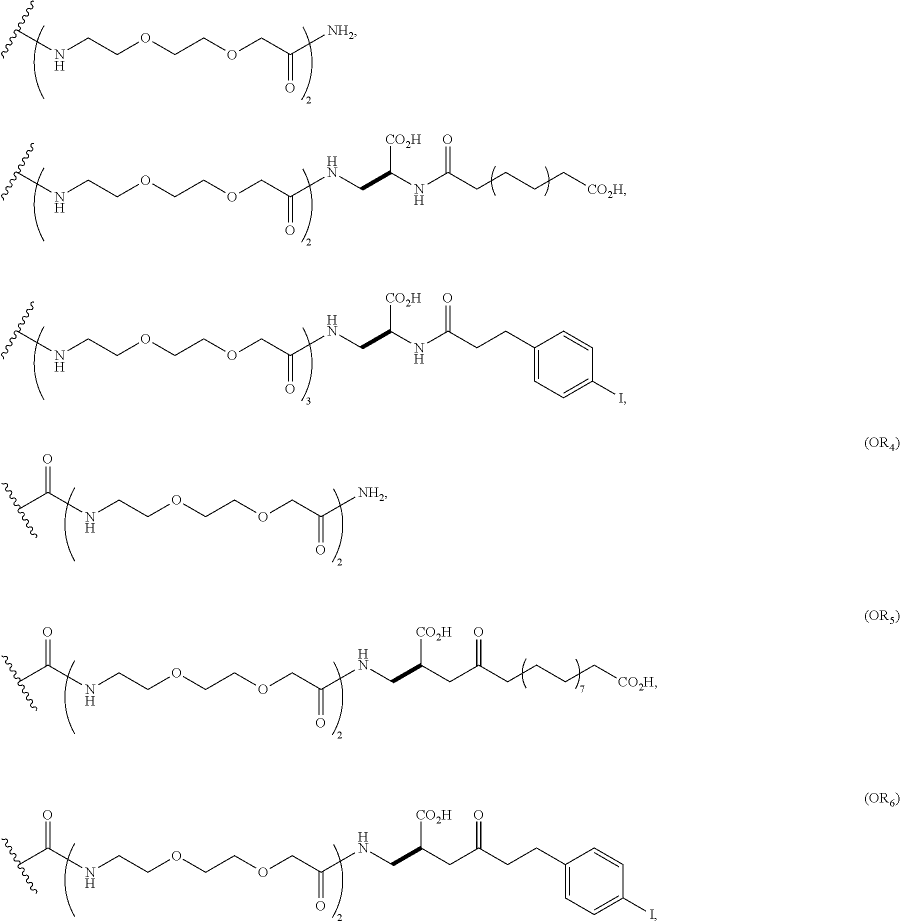

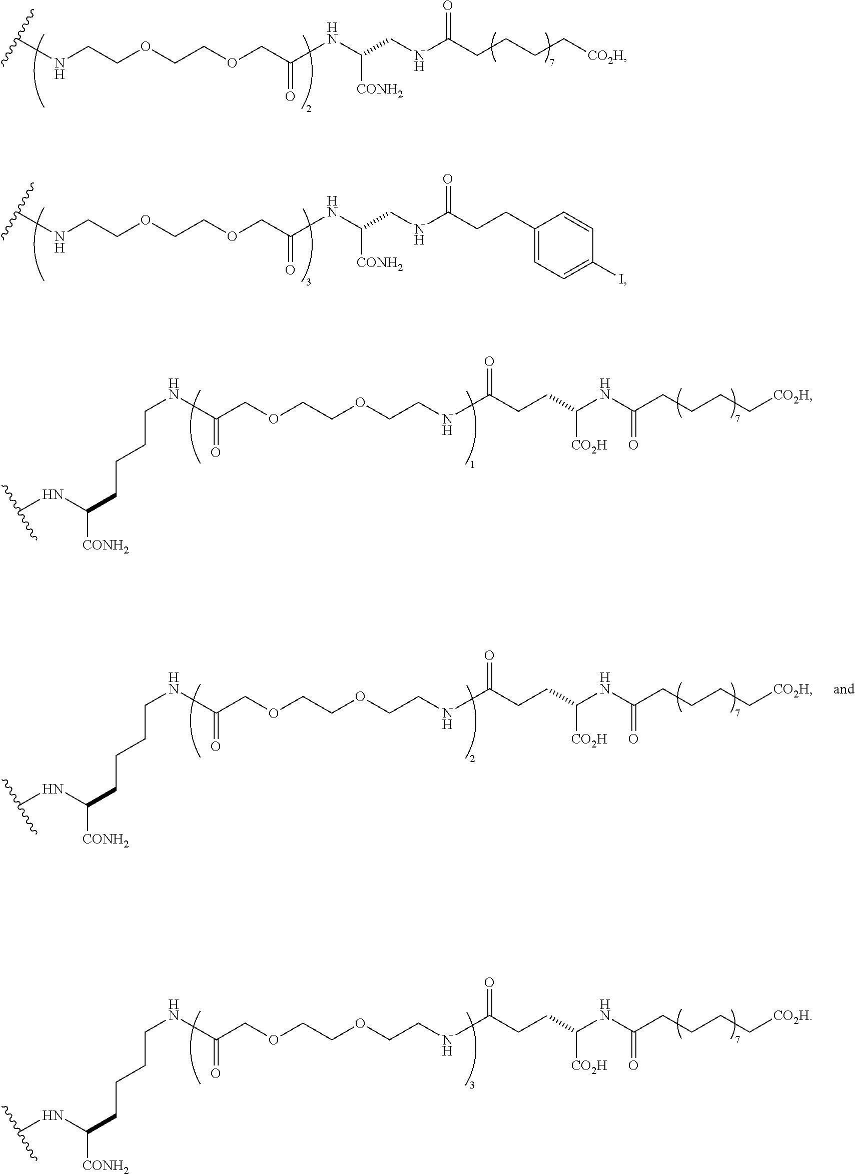

In certain embodiments, R₁₀ᵢ is selected from

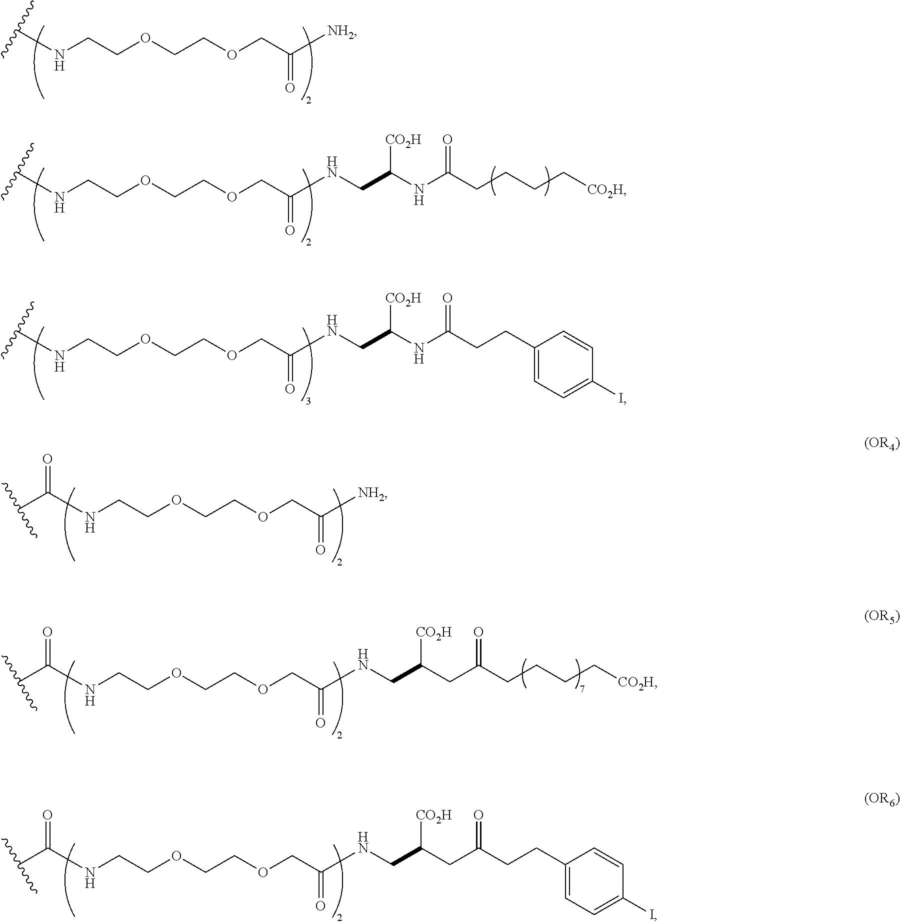

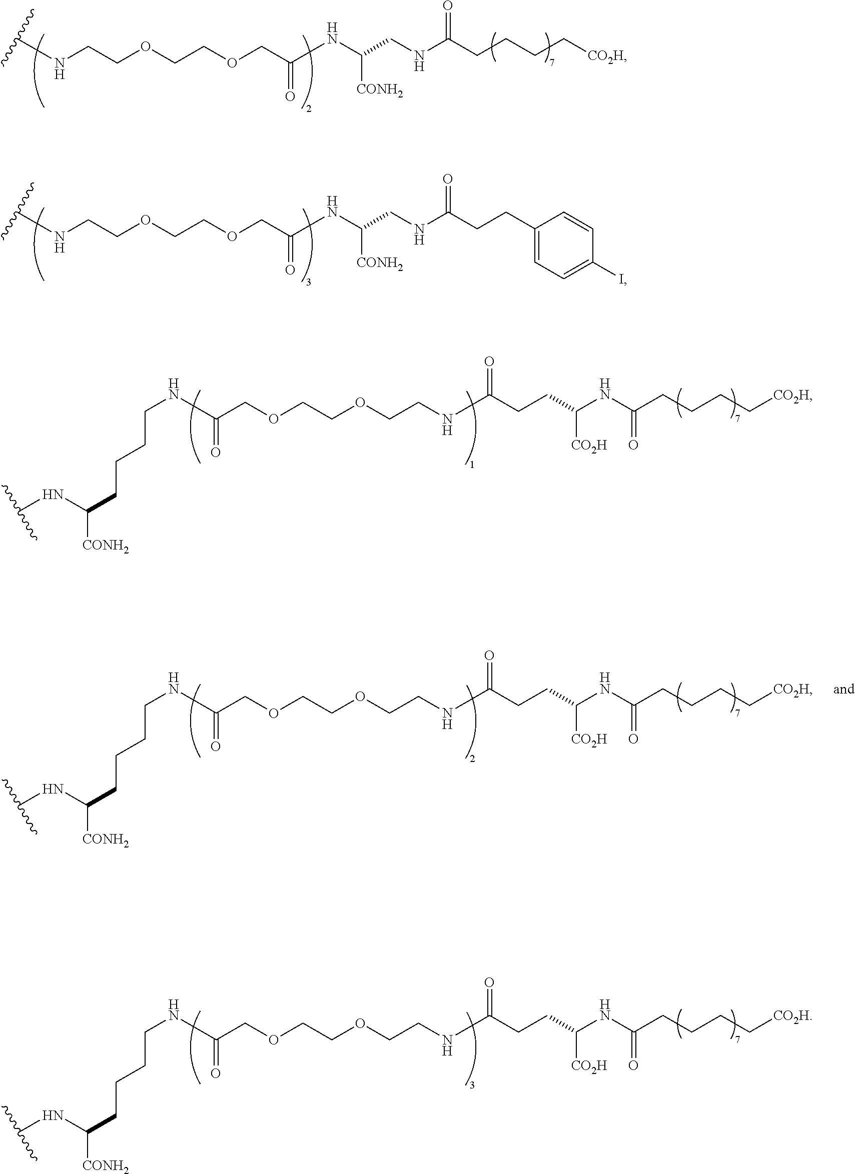

In certain embodiments, Xₐₐ¹⁰ is an optionally substituted amino acid residue selected from Phe, Tyr, Trp, homophenylalanine (Hph), homotyrosine (Hty), Bip, (α-MeBip, 4-phenyl-3-pyridylalanine, 4-phenyl-4-pyridylalanine, α-MeHph, α-MeTyr, α-MeHty, Tyr(O-phenyl), Phe(4-S-phenyl), Phe(4—SO₂—NH-phenyl), Phe(4-CO—NH-phenyl), Cys(S-phenyl), Cys(S-phenyl[2,3,4,5,6-F₅]), Cys(S-pheny[2,3,4,5-F₄]-4-phenyl[2′,3′,4′,5′,6′-F₅]), Cys(S-pheny[2,3,4,5-F₄]-4-SO₂-phenyl[2′,3′,4′,5′,6′-F₅]), Cy(SO₂—NH-phenyl), Dap(3-C[═O]-phenyl), Dap(3-[C═O]-pyridyl), Asp(3-NH-phenyl), and Asp(3-NH-pyridyl).

In certain embodiments, Xₐₐ¹¹ is an optionally substituted amino acid residue selected from Phe, Tyr, Trp, homophenylalanine (Hph), homotyrosine (Hty), Bip, (α-MeBip, 4-phenyl-3-pyridylalanine, 4-phenyl-₄-pyridylalanine, α-MeHph, α-MeTyr, α-MeHty, Tyr(O-phenyl), Phe(4-S-phenyl), Phe(4—SO₂—NH-phenyl), Phe(4-CO—NH-phenyl), Cys(S-phenyl), Cys(S-phenyl[2,3,4,5,6-F₅]), Cys(S-pheny[2,3,4,5-F₄]-4-phenyl[2′,3′,4′,5′,6′-F₅]), Cys(S-pheny[2,3,4,5-F₄]-4—SO₂-phenyl[2′,3′,4′,5′,6′-F₅]), Cy(SO₂—NH-phenyl), Dap(3-C[═O]-phenyl), Dap(3-[C═O]-pyridyl), Asp(3-NH-phenyl), and Asp(3-NH-pyridyl).

In certain embodiments, R_{XN} is —N(Rx)₂, wherein each Rx is H.

In certain embodiments, R_{XN} is —N(Rx)₂, wherein one occurrence of Rx is hydrogen and the other occurrence is an amino acid residue Xₐₐ⁰.

In certain embodiments, R_{YC} is —C(O)(NR_{Y})₂, wherein each R_{Y} is H.

In certain embodiments, R_{YC} is —C(O)(NR_{Y})₂;

one occurrence of R_{Y} is hydrogen and the other occurrence of R_{Y} is —L₄-L₅-L₆-L₇-R_{Y}′,

R_{Y}′ is (C₁-C₂₀ alkyl)-CO₂H or optionally substituted (C₁-C₆ alkyl)-aryl;

L₄ is absent or present and when present is a linker that comprises an amino acid moiety;

L₅ is absent or present and when present is a linker that comprises an amino acid moiety;

L₆ is absent or present and when present is a linker that comprises an ether moiety; and

L₇ is a linker that comprises an amino acid moiety.

In certain embodiments, L₄ is present and is

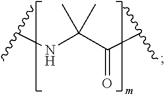

and m is 1-6.

In certain embodiments, L₅ is present and is

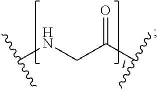

and l is 1-6.

In certain embodiments, L₅ is present and is

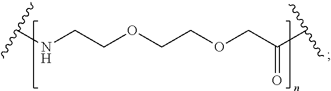

and n is 1-6.

In certain embodiments, L₆ is

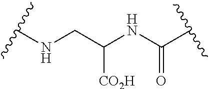 [SMILES: *NCC(NC(*)=O)C(=O)O]

or

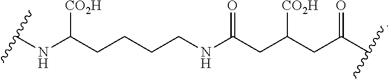 [SMILES: *NC(CCCCNC(=O)CC(CC(*)=O)C(=O)O)C(=O)O]

In certain embodiments, R_{Y}′ is (C₁₀-C₁₆ alkyl)-CO₂H.

In certain embodiments, R_{Y}′ is

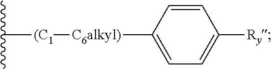

and R_{Y}″ is a halo.

In certain embodiments, R_{Y}″ is a I.

In certain embodiments, one occurrence of R_{Y} is hydrogen and the other occurrence is selected from

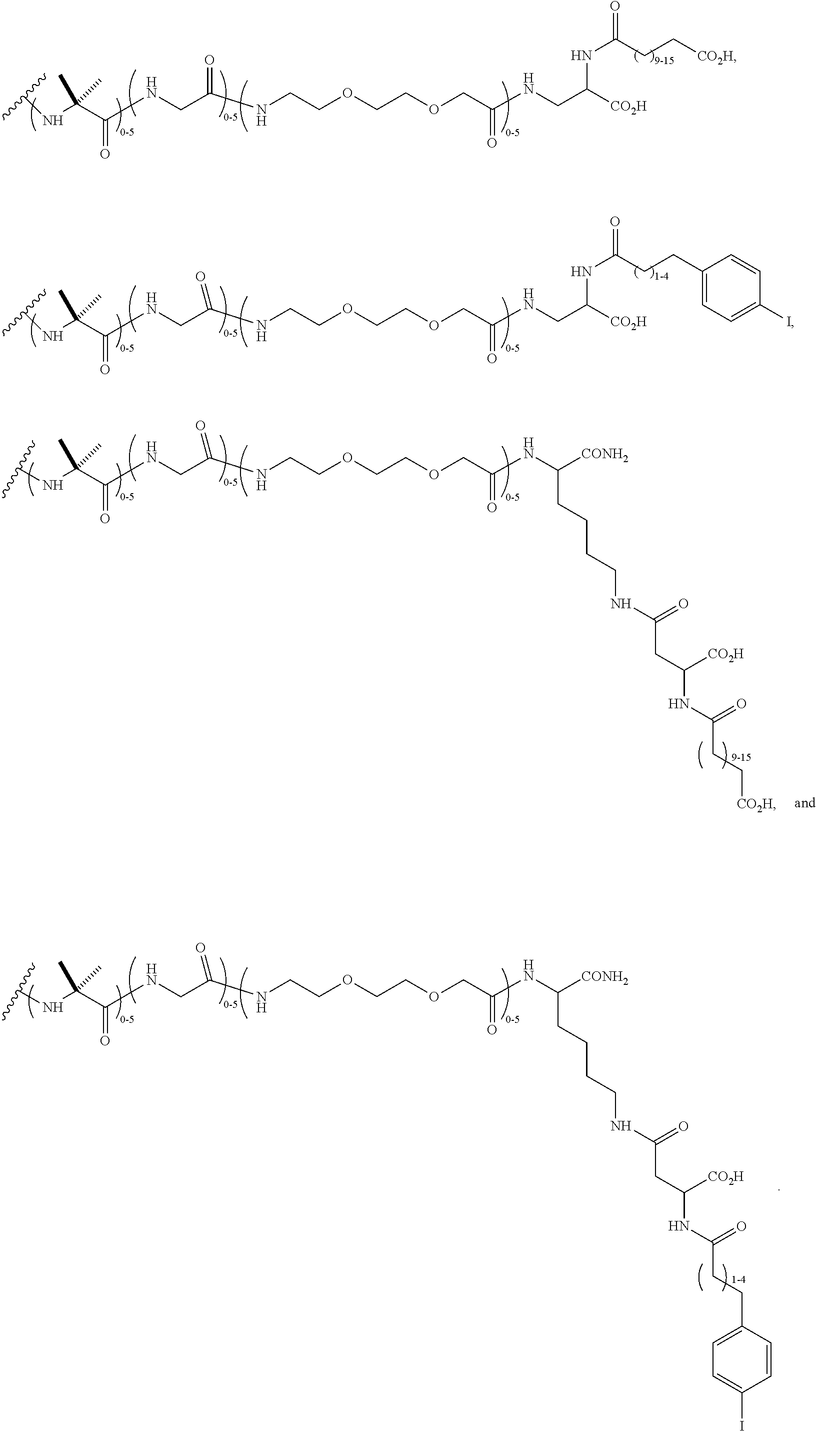

In certain embodiments, the polypeptide is selected from the polypeptides recited in Table 4.

In certain embodiments, the polypeptide is selected from the polypeptides recited in Table 5.

In certain embodiments, the polypeptide is selected from the polypeptides recited in Table 6.

In certain embodiments, the polypeptide is represented by:

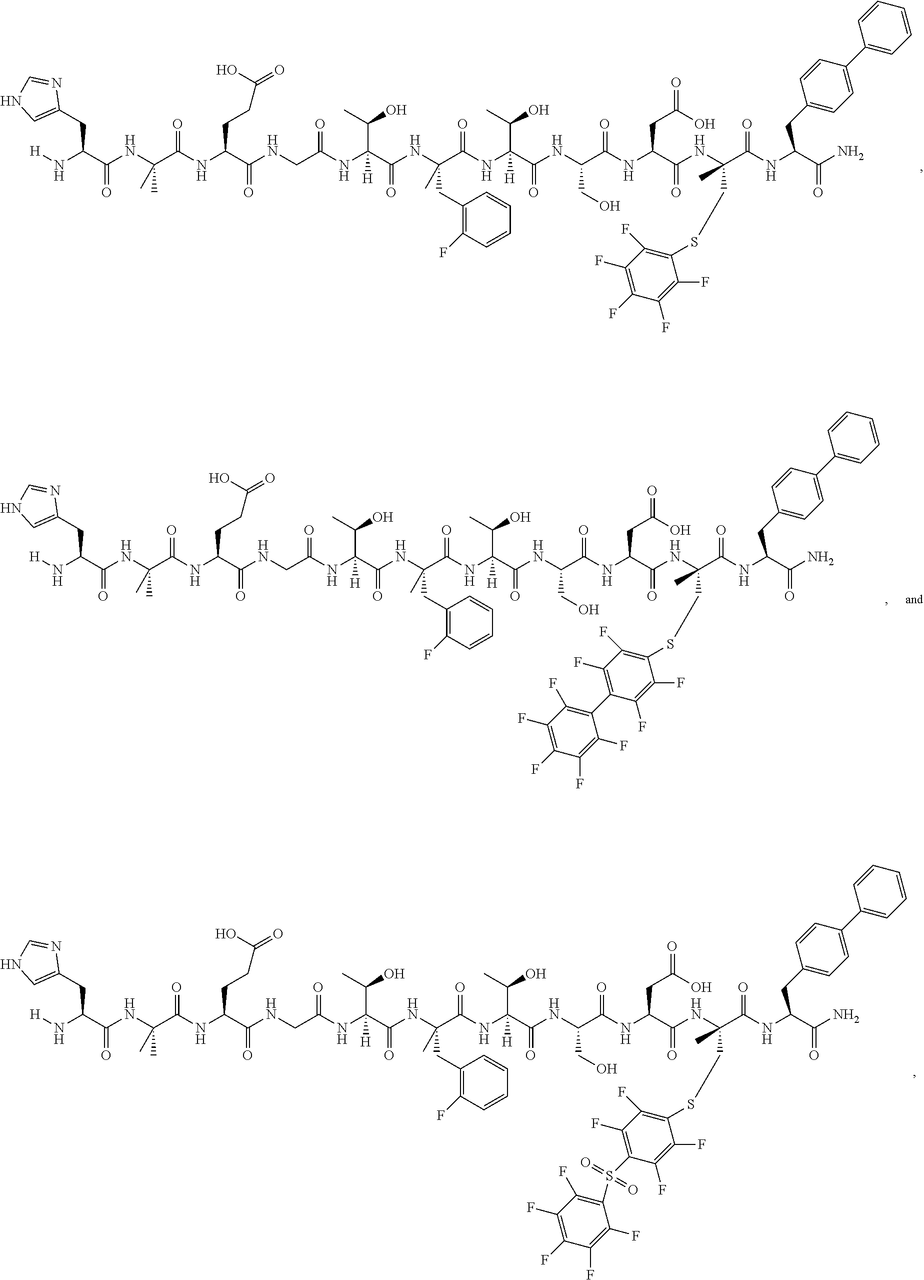

In certain embodiments, the polypeptide is represented by:

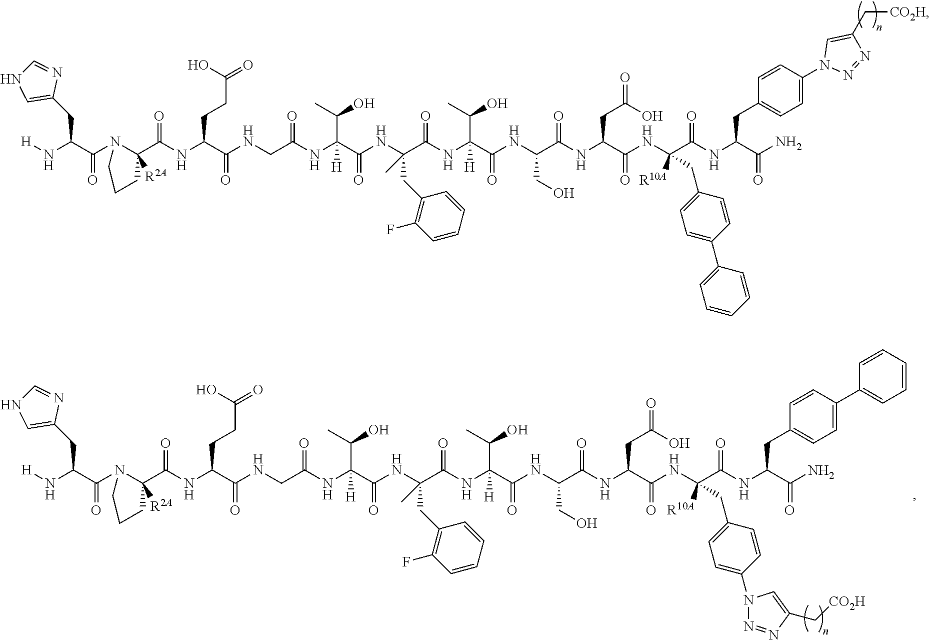

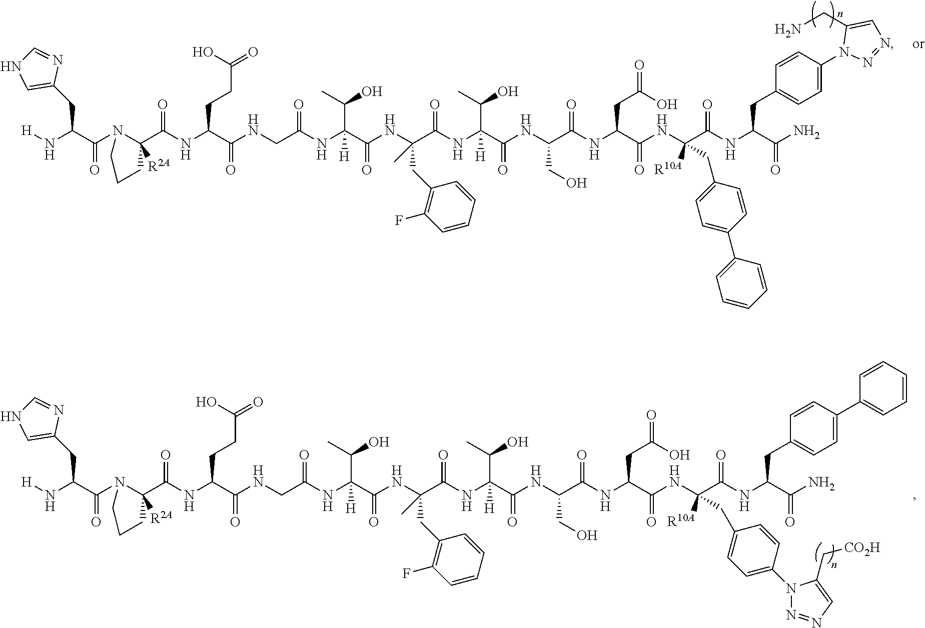

wherein

each n is independently 10 to 20 (e.g., 15);

each R^{2A} is independently —H or alkyl; and.

each R^{10A} is independently —H or alkyl.

In certain embodiments, each n is independently 10 to 20 (e.g., 15); each R^{2A} is independently —H or —CH₃; and each R^{10A} is independently —H or —CH₃.

In certain embodiments, the polypeptide is represented by:

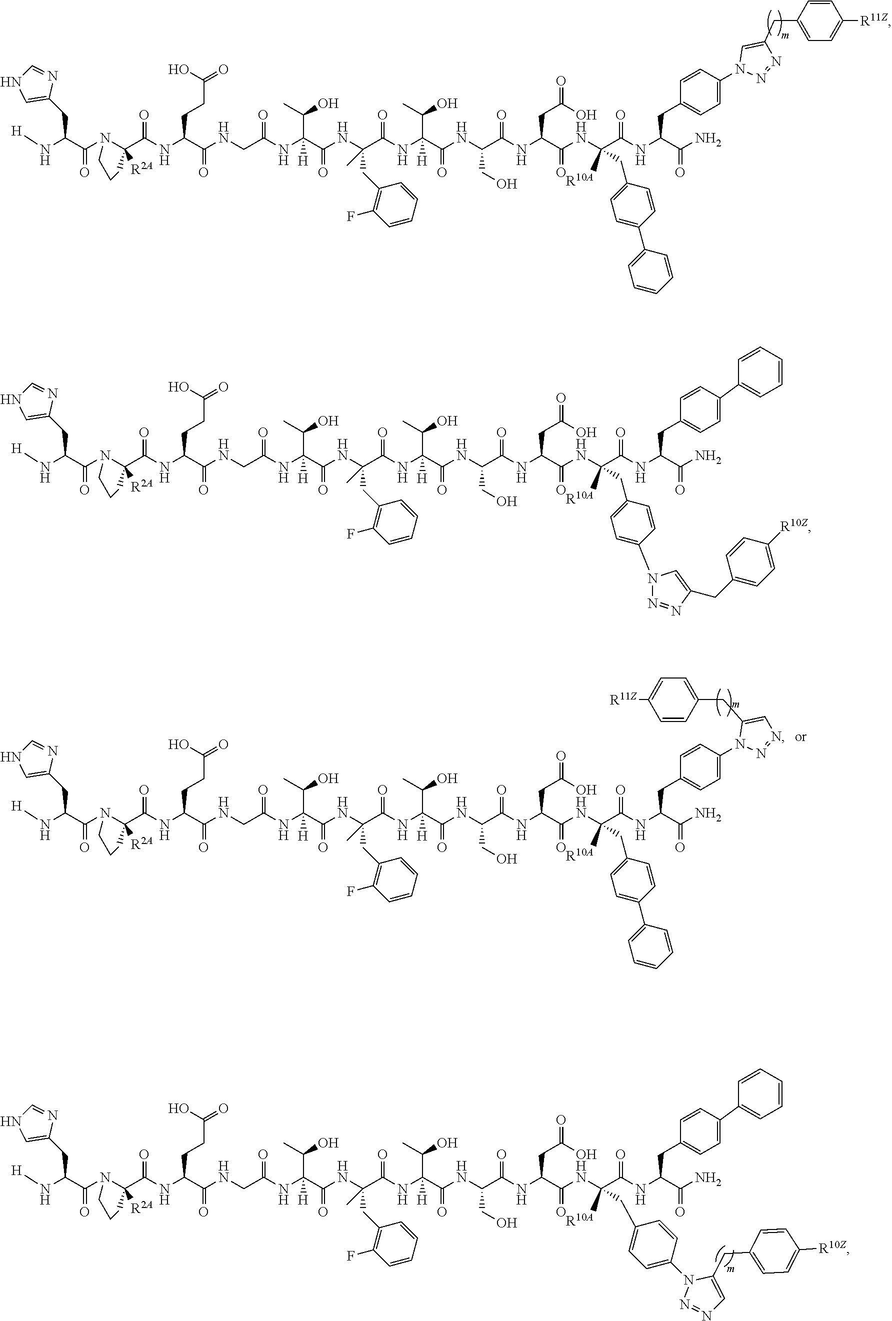

wherin

each m is independently 1 to 10;

each R^{2A} is independently —H or alkyl;

each R^{10A} is independently —H or alkyl;

each R^{10Z} is independently halo (e.g., I); and

each R^{11Z} is independently halo (e.g., I).

In certain embodiments, each n is independently 1 to 10; each R^{2A} is independently —H or —CH₃; each R^{10A} is independently —H or —CH₃; each R^{10Z} is independently halo (e.g., I); and each R^{1aZ} is independently halo (e.g., I).

In certain embodiments, each R^{10Z} is I; and each R^{11Z} is I.

In certain embodiments, the polypeptide is represented by:

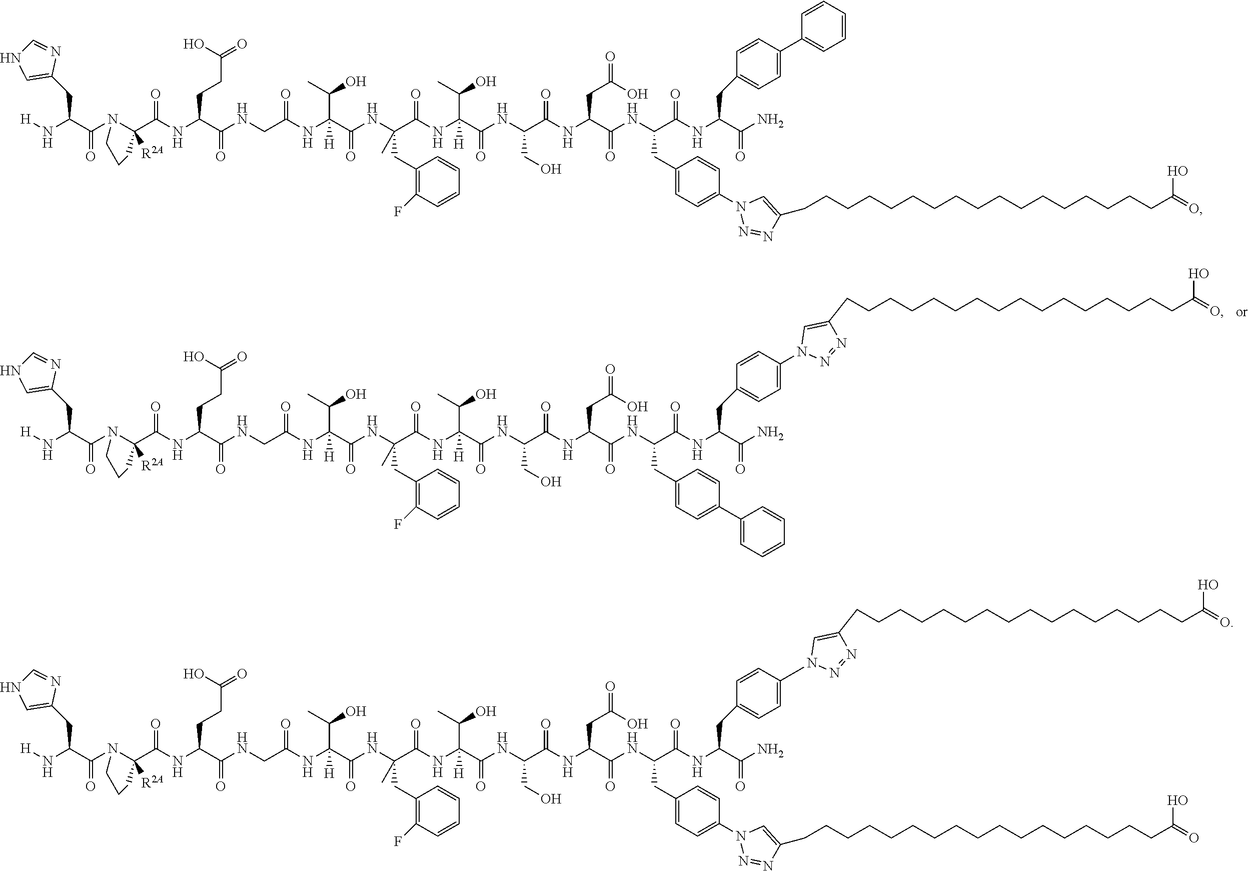

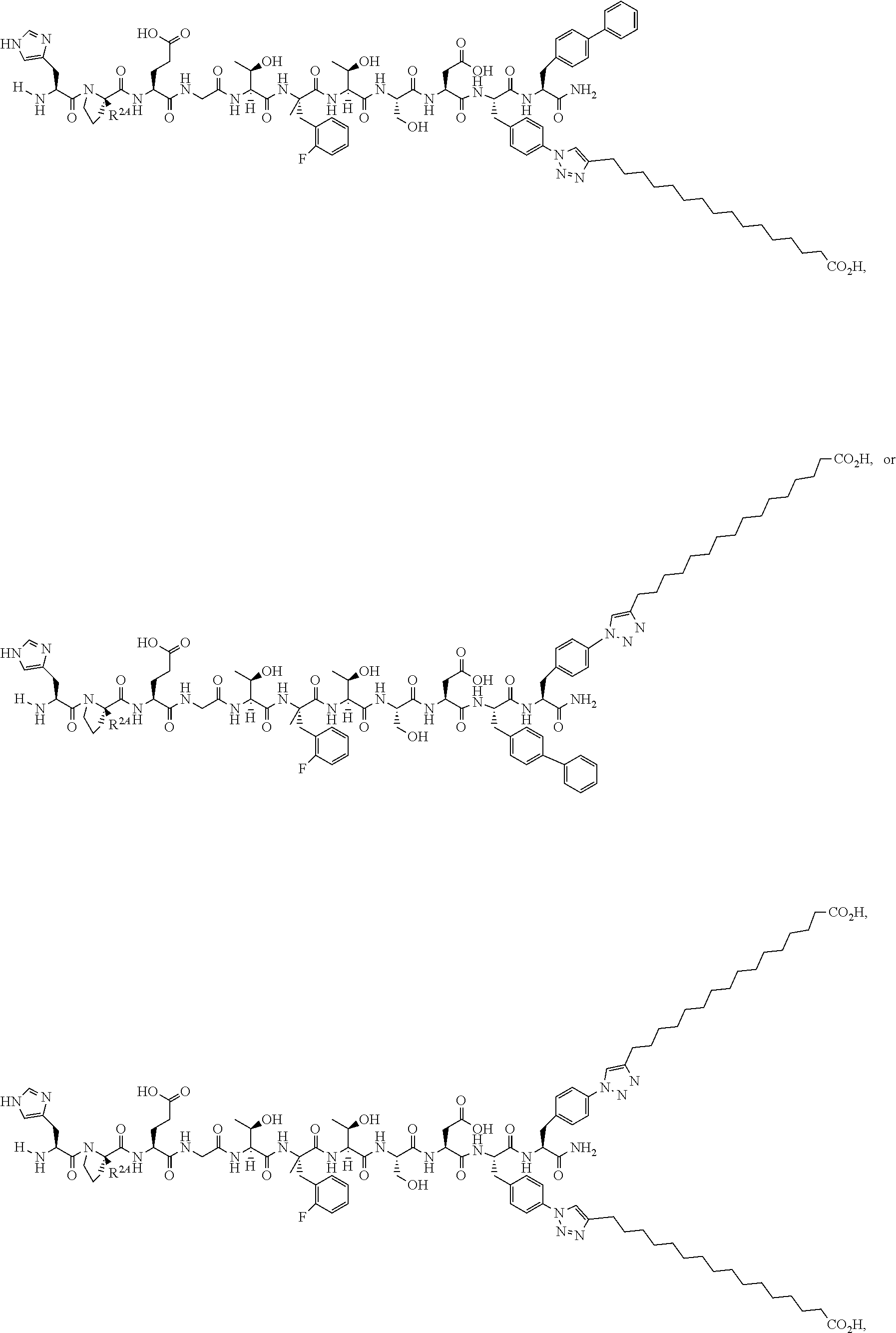

In certain embodiments, the polypeptide is represented by:

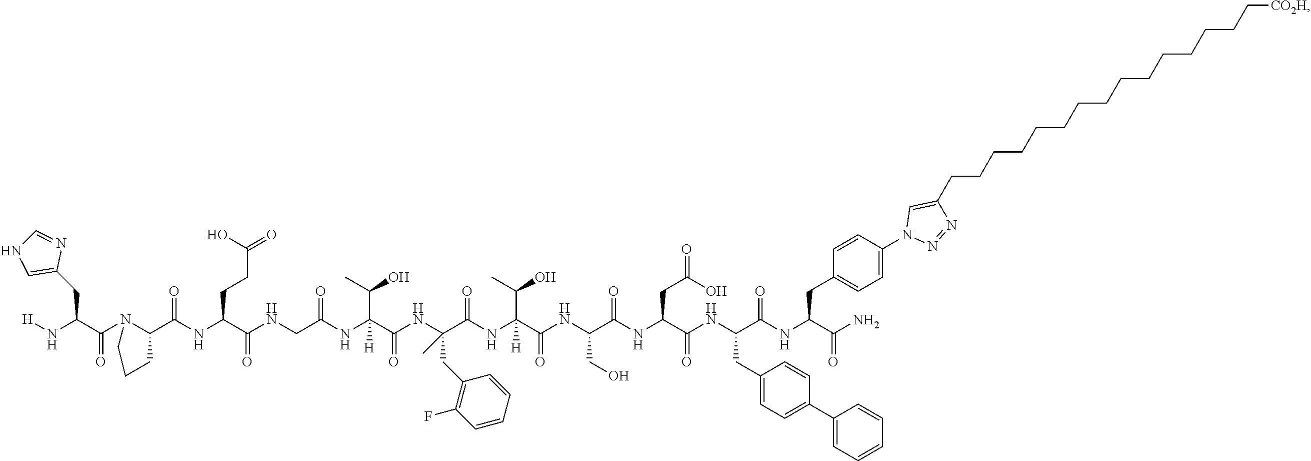 [SMILES: C[C@@H](O)[C@H](NC(=O)CNC(=O)[C@H](CCC(=O)O)NC(=O)[C@@H]1CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-c2ccccc2)cc1)C(=O)N[C@@H](Cc1ccc(-n2cc(CCCCCCCCCCCCCCCC(=O)O)nn2)cc1)C(N)=O)[C@@H](C)O]

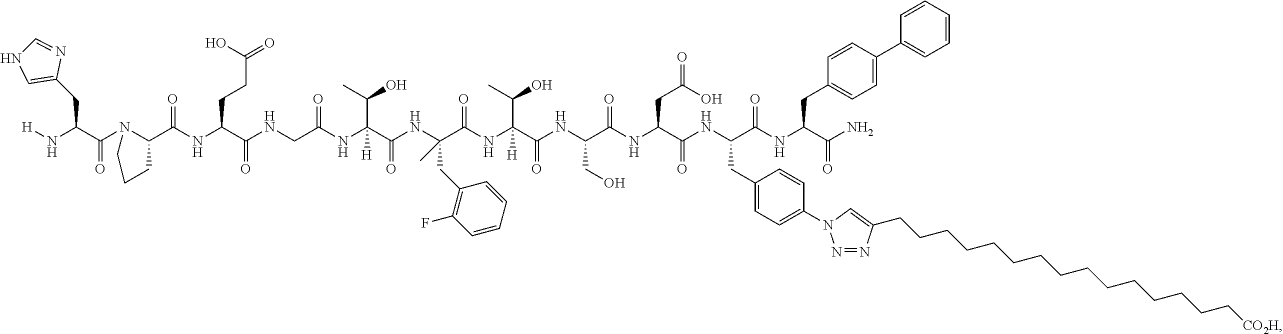 [SMILES: C[C@@H](O)[C@H](NC(=O)CNC(=O)[C@H](CCC(=O)O)NC(=O)[C@@H]1CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-n2cc(CCCCCCCCCCCCCCCC(=O)O)nn2)cc1)C(=O)N[C@@H](Cc1ccc(-c2ccccc2)cc1)C(N)=O)[C@@H](C)O]

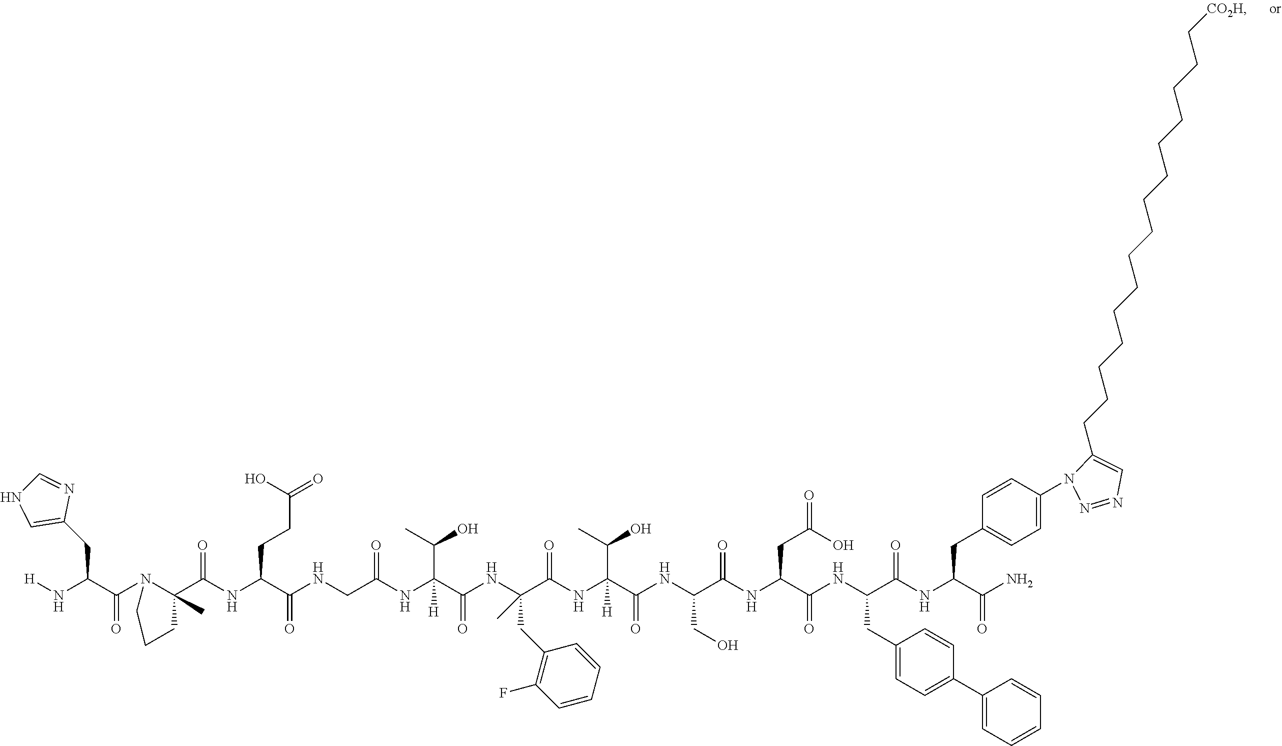 [SMILES: C[C@@H](O)[C@H](NC(=O)CNC(=O)[C@H](CCC(=O)O)NC(=O)[C@]1(C)CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-c2ccccc2)cc1)C(=O)N[C@@H](Cc1ccc(-n2nncc2CCCCCCCCCCCCCCCC(=O)O)cc1)C(N)=O)[C@@H](C)O]

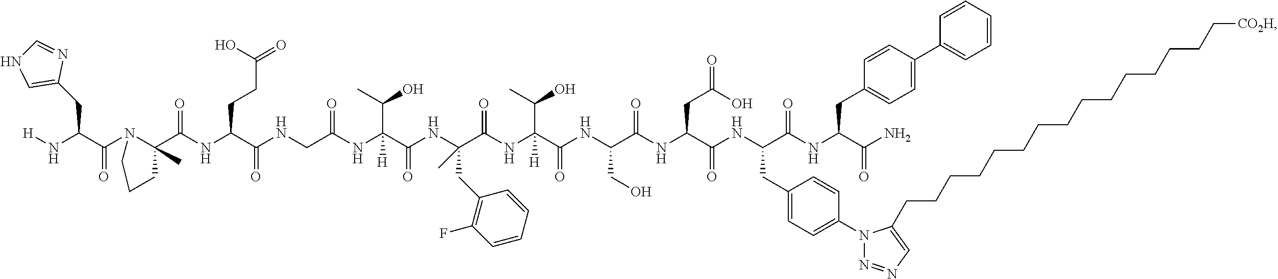 [SMILES: C[C@@H](O)[C@H](NC(=O)CNC(=O)[C@H](CCC(=O)O)NC(=O)[C@]1(C)CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-n2nncc2CCCCCCCCCCCCCCCC(=O)O)cc1)C(=O)N[C@@H](Cc1ccc(-c2ccccc2)cc1)C(N)=O)[C@@H](C)O]

In certain embodiments, the polypeptide is represented by:

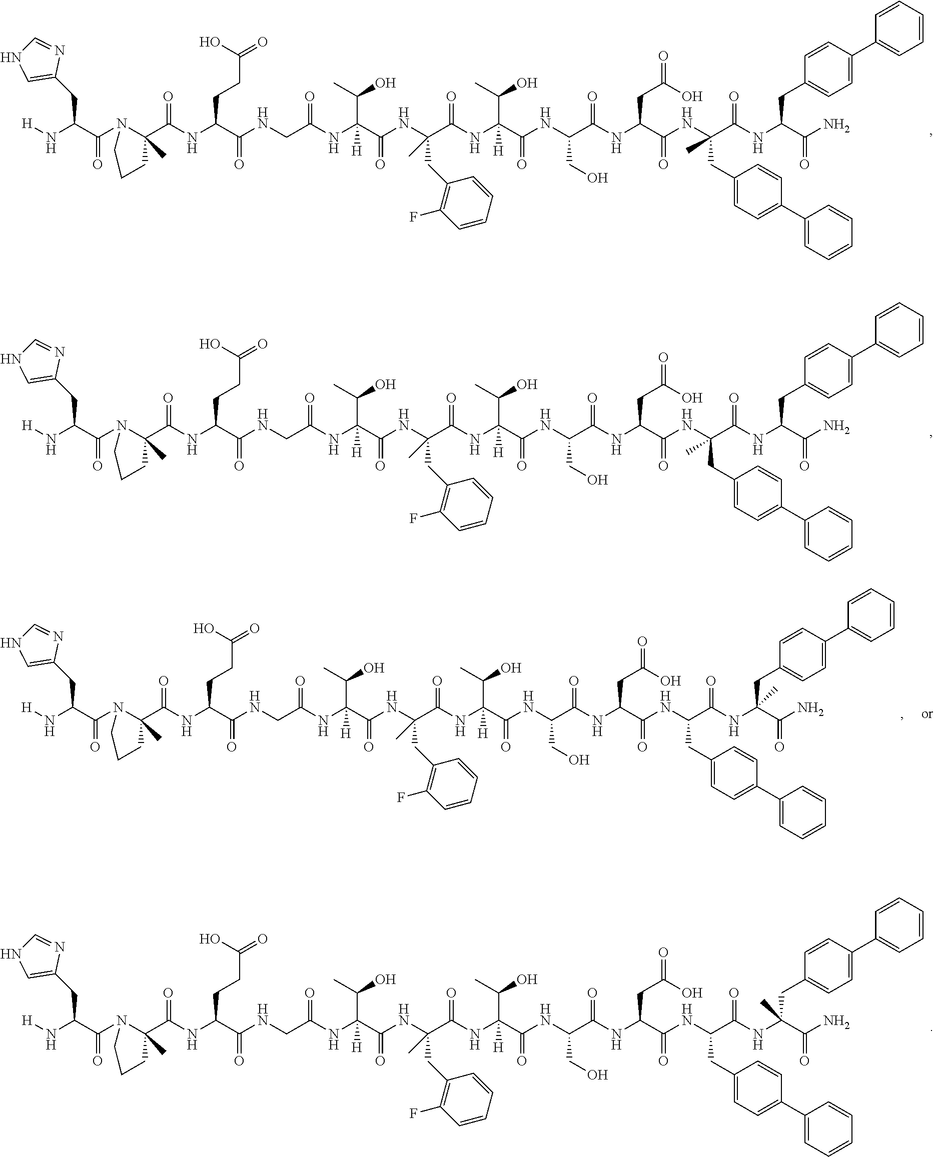

In certain embodiments, S-Aryl1 has the structure:

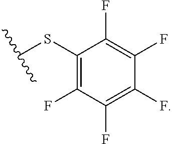 [SMILES: *Sc1c(F)c(F)c(F)c(F)c1F]

In certain embodiments, S-Aryl12 has the structure:

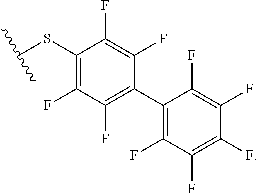 [SMILES: *Sc1c(F)c(F)c(-c2c(F)c(F)c(F)c(F)c2F)c(F)c1F]

In certain embodiments, S-Aryl3 has the structure:

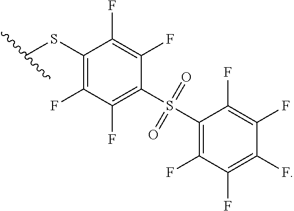 [SMILES: *Sc1c(F)c(F)c(S(=O)(=O)c2c(F)c(F)c(F)c(F)c2F)c(F)c1F]

In certain embodiments, R_{10d} or R₁₀ᵢ are selected from a group consisting of hydroxyl, amino, carboxyl, azido, alkynyl, or a methyl group that is substituted by thiol, hydroxyl, amino, carboxyl, azido or alkynyl as well as further conjugation through such substituents (e.g., thioethers, ethers, amines, esters, triazines) to functionalities that enhance the pharmacokinetic properties by binding to serum albumin in a manner exemplified by GLP-1 peptide agonists such as Liraglutide and Semaglutide (17-19) by incorporation of fatty acids (e.g., C₁₆ or C₁₈) or yet by other known serum albumin binding moieties as represented by aryl-halides, as shown by, but not limited to, phenyl-iodide (20) and exemplified below.

In certain embodiments, R_{10d} or R₁₀ᵢ is selected from:

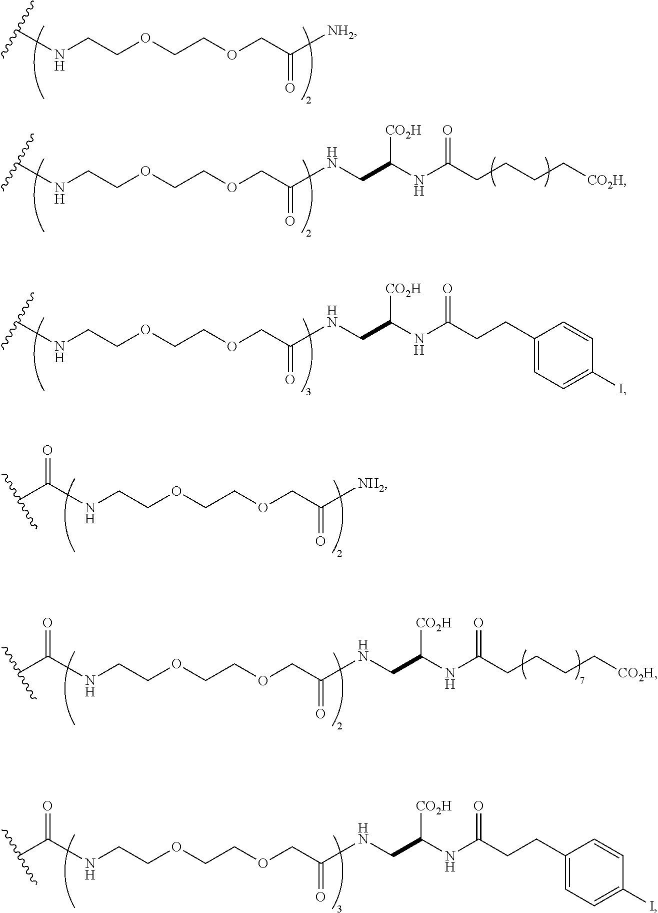

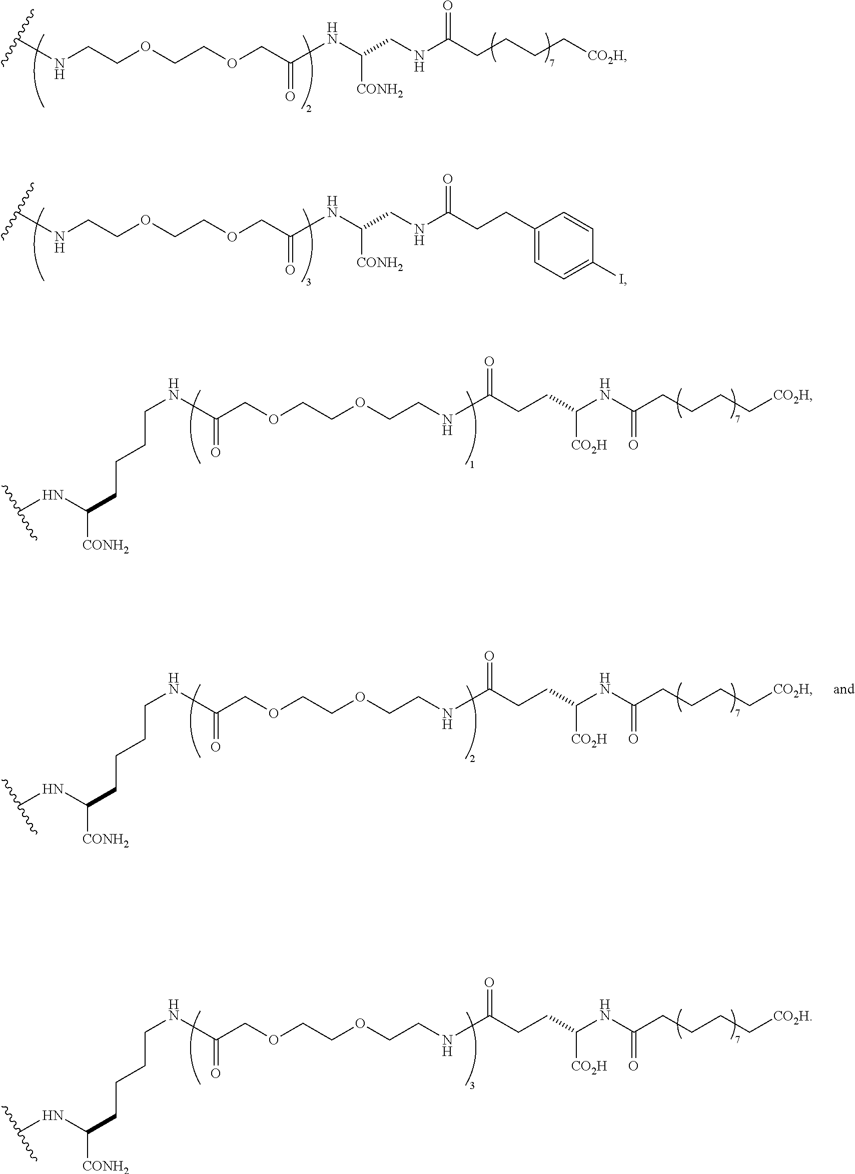

In certain embodiments, R_{YC} is —C(O)NHR_{Y}, wherein NHR_{Y} is selected from:

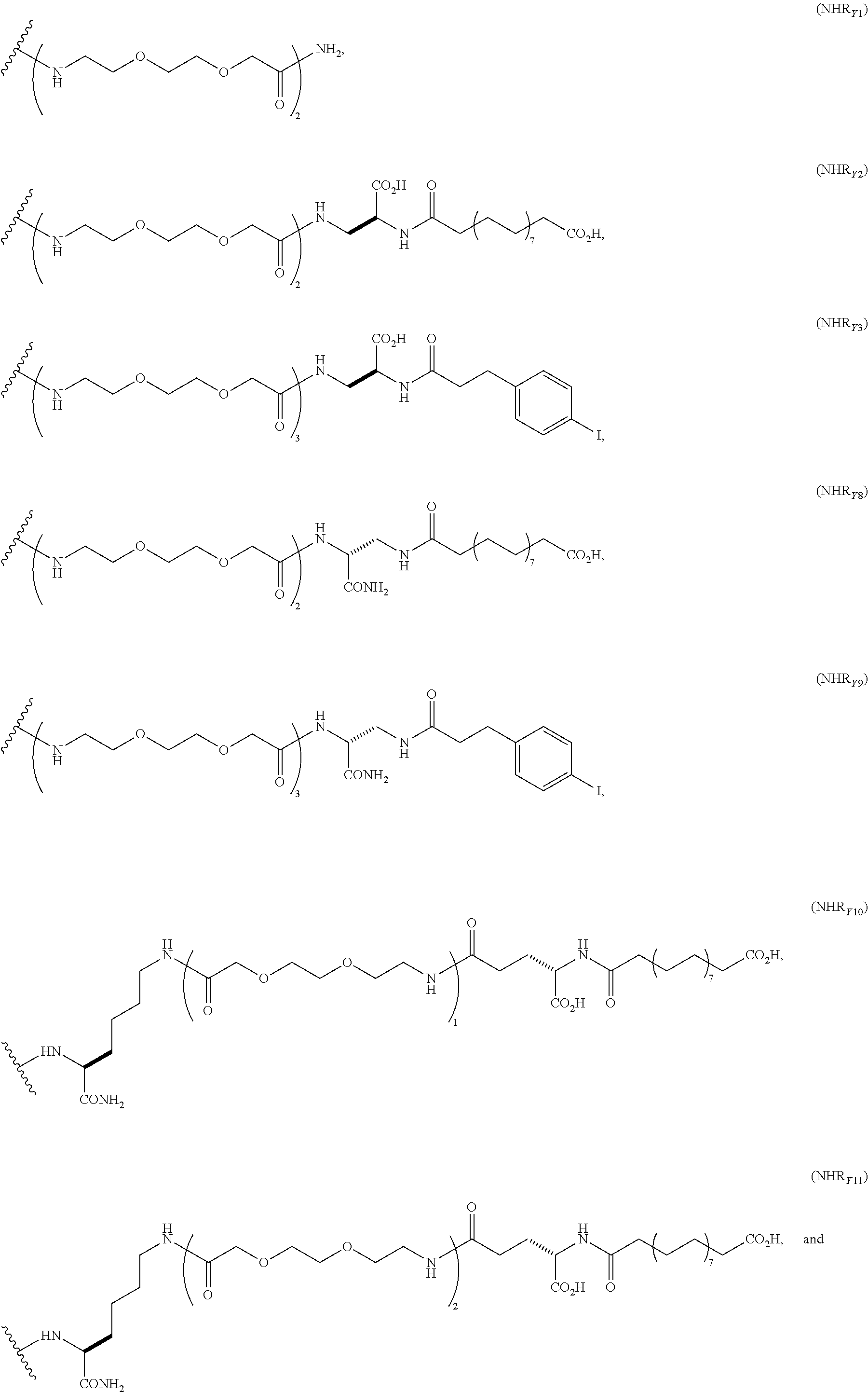

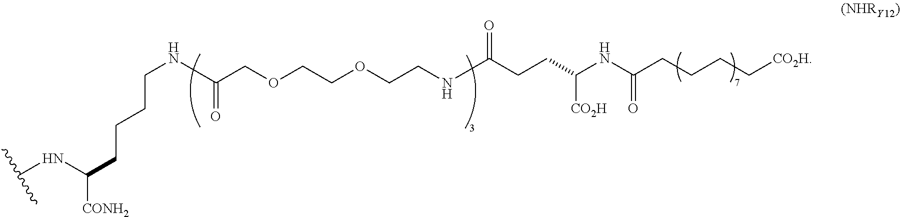

In certain embodiments, R_{YC} is —C(O)NHR_{Y}, wherein NHR_{Y} is selected from:

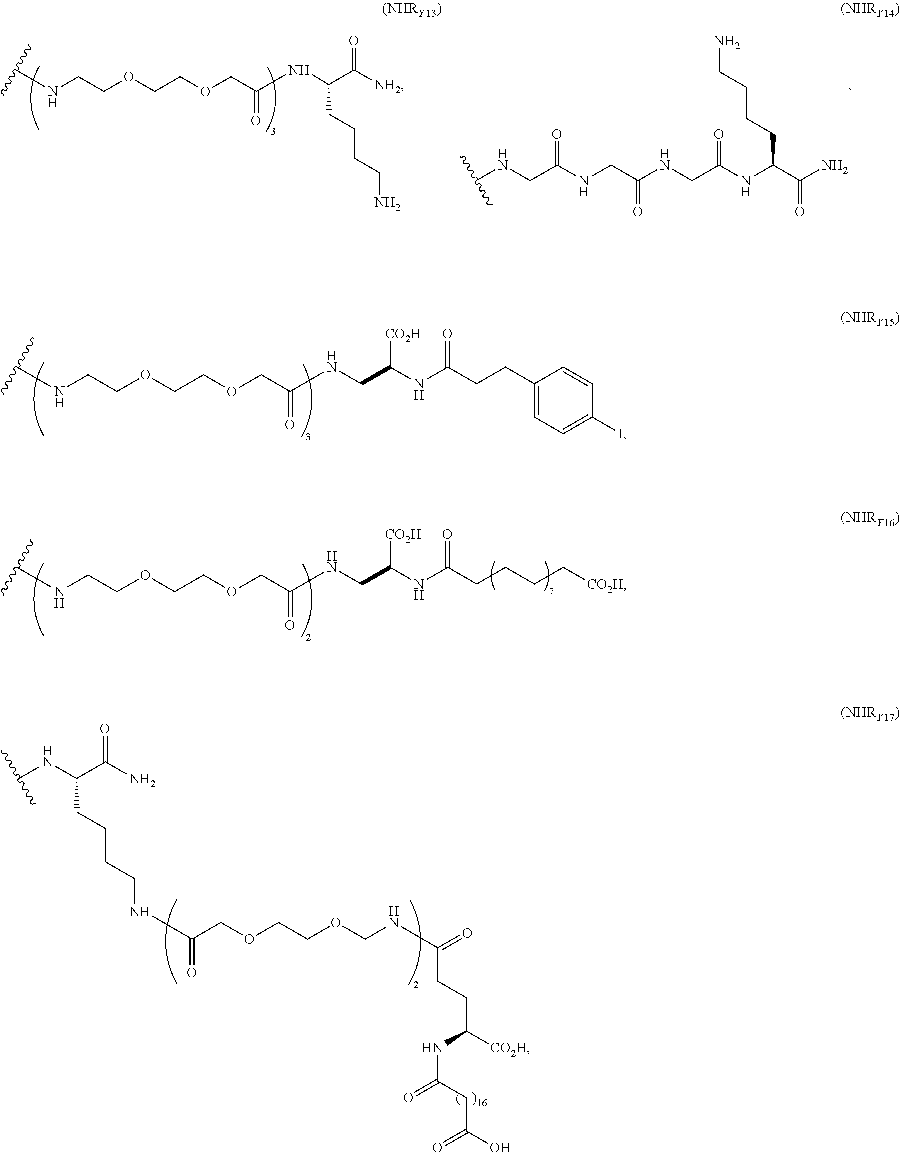

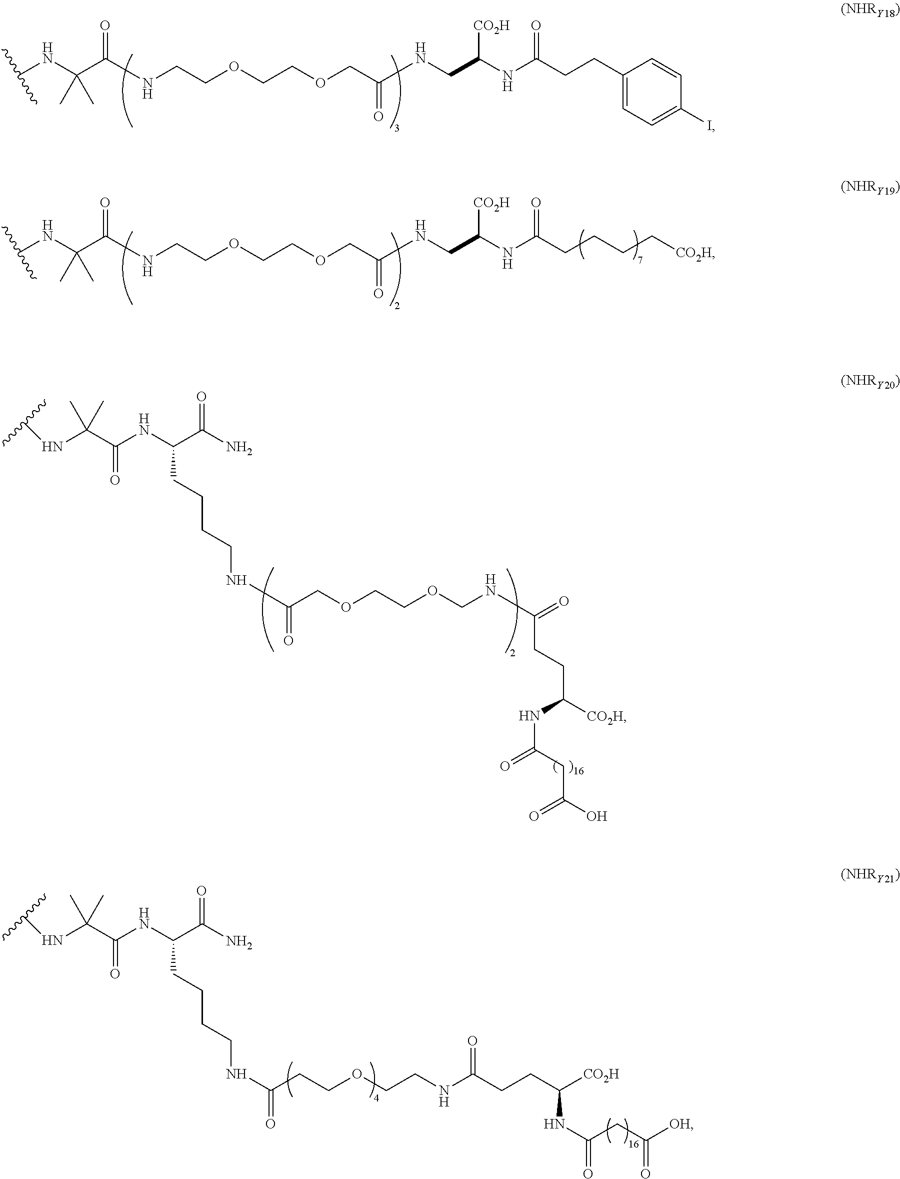

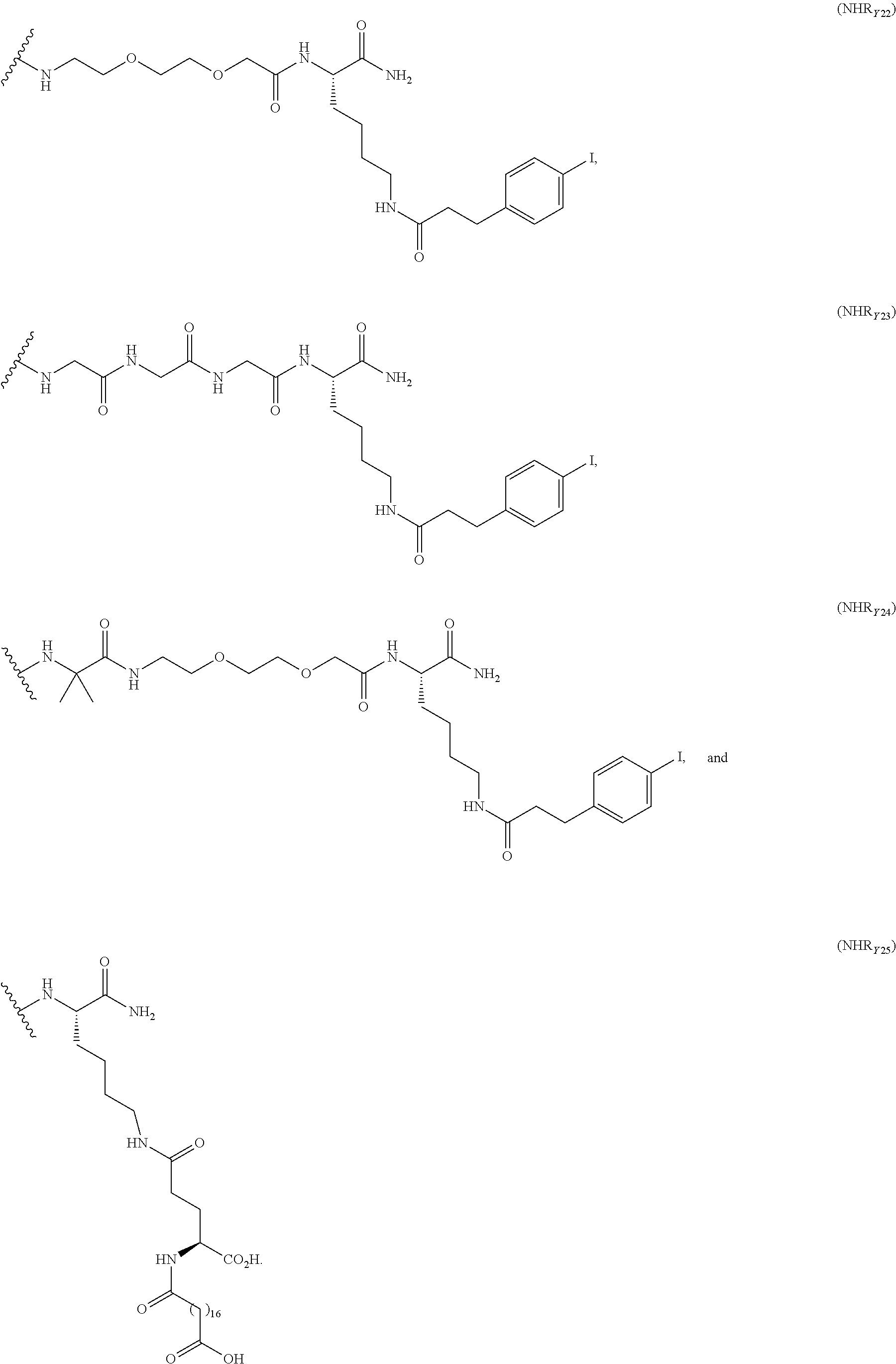

In certain embodiments, R_{YC} is —C(O)NHR_{Y}, wherein NHR_{Y} is selected from:

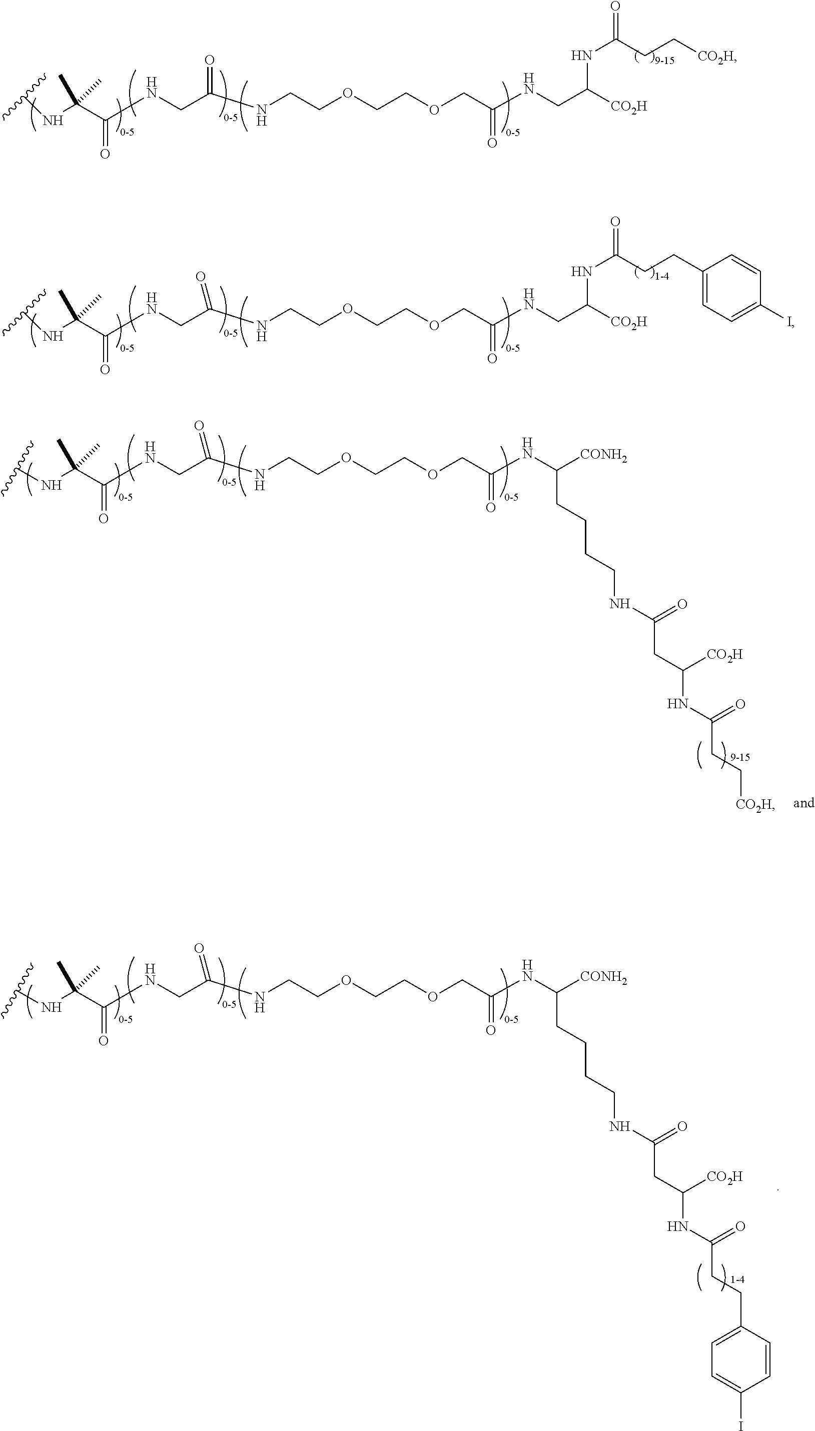

In certain embodiments, Xₐₐ¹⁰ is an optionally substituted amino acid residue selected from Phe, Tyr, Trp, homophenylalanine (Hph), homotyrosine (Hty), Bip, (α-MeBip, 4-phenyl-₃-pyridylalanine, 4-phenyl-₄-pyridylalanine, α-MeHph, α-MeTyr, α-MeHty, Tyr(O-phenyl), Phe(4-S-phenyl), Phe(4—SO₂—NH-phenyl), Phe(4-CO—NH-phenyl), Cys(S-phenyl), Cys(S-phenyl[2,3,4,5,6-F₅]), Cys(S-pheny[2,3,4,5-F₄]-4-phenyl[2′,3′,4′,5′,6′-F₅]), and Cys(S-pheny[2,3,4,5-F₄]-4—SO₂-phenyl[2′,3′,4′,5′,6′-F₅]).

In certain embodiments, Xₐₐ¹⁰ is an optionally substituted Cy(SO₂—NH-phenyl), i.e. where the —SO₃H group of cysteic acid is replaced with an —SO₂—NH-phenyl.

In certain embodiments, Xₐₐ¹⁰ is an optionally substituted amino acid residue selected from Dap(3-C[═O]-phenyl), i.e. where the amino group of the sidechain is replaced with —NHC(O)-phenyl, Dap(3-[C═O]-pyridyl), i.e. where the amino group of the sidechain is replaced with —NHC(O)-4-pyridinyl, Asp(3-NH-phenyl), i.e. where the carboxyl group of the sidechain is replaced with —C(O)NH-phenyl, and Asp(3-NH-pyridyl), i.e. where the carboxyl group of the sidechain is replaced with —C(O)NH-4-pyridinyl.

In certain embodiments, Xₐₐ¹¹ is an optionally substituted Cy(SO₂—NH-phenyl), i.e. where the —SO₃H group of cysteic acid is replaced with an —SO₂—NH-phenyl.

In certain embodiments, Xₐₐ¹¹ is an optionally substituted amino acid residue selected from Dap(3-C[═O]-phenyl), i.e. where the amino group of the sidechain is replaced with —NHC(O)-phenyl, Dap(3-[C═O]-pyridyl), i.e. where the amino group of the sidechain is replaced with —NHC(O)-4-pyridinyl, Asp(3-NH-phenyl), i.e. where the carboxyl group of the sidechain is replaced with —C(O)NH-phenyl, and Asp(3-NH-pyridyl), i.e. where the carboxyl group of the sidechain is replaced with —C(O)NH-4-pyridinyl.

In certain embodiments, Xₐₐ² and/or Xₐₐ¹¹ are D-amino acid residues.

In certain embodiments, each of Xₐₐ¹ to Xₐₐ¹¹ is an L-amino acid residue.

In certain embodiments, the amino acid residue, when substituted, is substituted with alkyl or halo.

In certain embodiments, the amino acid residue, when substituted, is substituted with alkyl, hydroxyl, or halo.

In certain embodiments, any one of the amino acid residue are selected from natural amino acids.

In certain embodiments, any one of the amino acid residues is selected from unnatural amino acids.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C—or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Any compound of the invention can also be radiolabed for the preparation of a radiopharmaceutical agent.

### Methods of Treatment

One aspect of the invention relates to a method of treating or preventing a disease or disorder at least partially mediated by glucagon-like peptide 1 in a subject in need thereof comprising administering to the subject an effective amount of a polypeptide of sequence (I).

In certain embodiments, a method of treating or preventing diabetes in a subject in need thereof comprising administering to the subject an effective amount of a polypeptide of sequence (I).

In certain embodiments, the diabetes is type-II diabetes.

In certain embodiments, a method of treating, preventing, or delaying the onset of complications related to diabetes, including macrovascular and microvascular complications such as retinopathy, neuropathy, nephropathy and delayed wound healing, and related diseases such as insulin resistance (impaired glucose homeostasis), hyperglycemia, hyperinsulinemia, elevated blood levels of fatty acids or glycerol, obesity, hyperlipidemia including hypertriglyceridemia, Syndrome X, atherosclerosis and hypertension, and for increasing high density lipoprotein levels.

In certain embodiments, the method further comprising administering an anti-diabetic agent.

In certain embodiments, the method further comprising administering a lipid lowering agent, which may be applied in the setting of human immunodeficiency virus (HIV) and its treatment.

In certain embodiments, a method of treating or preventing obesity or related metabolic disorders such as polycystic ovarian disease (PCOS) in a subject in need thereof comprising administering to the subject an effective amount of a polypeptide of sequence (I).

In certain embodiments, the method further comprising administering an anti-obesity agent.

In certain embodiments, a method of treating or preventing cardiovascular disease in a subject in need thereof comprising administering to the subject an effective amount of a polypeptide of sequence (I).

In certain embodiments, the method further comprising administering an anti-hypertensive agent.

In certain embodiments, a method of treating or preventing a neurodegenerative disease in a subject in need thereof comprising administering to the subject an effective amount of a polypeptide of sequence (I).

In certain embodiments, the neurodegenerative disease is selected from Alzheimer's disease, Parkinson's disease, multiple sclerosis, amyotrophic lateral sclerosis, Huntington's disease, and prion diseases.

In certain embodiments, a method of treating or preventing a traumatic brain injury (TBI) in a subject in need thereof comprising administering to the subject an effective amount of a polypeptide of sequence (I).

In certain embodiments, a method of treating or preventing non-alcoholic steatohepatitis (NASH) in a subject in need thereof comprising administering to the subject an effective amount of a polypeptide of sequence (I).

In some embodiments of any one of the disclosed methods, the polypetide of sequence (I) is defined as:

R_{XN}—Xₐₐ¹—Xₐₐ²—Xₐₐ³—Xₐₐ⁴—Xₐₐ⁵—Xₐₐ⁶—Xₐₐ⁷—Xₐₐ⁸—Xₐₐ⁹—Xₐₐ¹⁰—Xₐₐ¹¹—R_{YC}

wherein

R_{XN} is the N-terminal group of Xₐₐ¹ selected from H (i.e., des-amino) and —N(Rx)₂, wherein Rx, independently for each occurrence, is H or an optionally substituted alkyl, arylalkyl, heteroarylalkyl, formyl, acetyl, alkanoyl, —C(O)-alkyloxy, —C(O)-aryloxy, —C(O)-aralkyloxy, —C(O)-heterocyclyloxy, —C(O)-heteroarylalkyloxy, —C(O)NH-alkyl, —C(O)NH-aryl, —C(O)NH— arylalkyl, —SO₂-heterocyclyl, —SO₂-alkyl, —SO₂-aryl, —SO₂-arylalkyl, —SO₂-heteroarylalkyl, —SO₂-heteroaryl, or ureido; or one occurrence of Rx is hydrogen and the other occurrence is an amino acid residue Xₐₐ⁰;

Xₐₐ⁰ is an optionally substituted amino acid residue selected from Gly, Pro, Arg, Glu, His, Phe and Trp;

Xₐₐ^{l} is an optionally substituted amino acid residue comprising an amino acid side chain that comprises an alkyl, aryl or heteroaryl;

Xₐₐ² is an optionally substituted amino acid residue selected from Gly, Aib, Ala, D-Ala, N-methyl-Ala, N-methyl-D-Ala, Pro, α-methyl-Pro, Val, D-Val, and D-His;

Xₐₐ³ is an optionally substituted amino acid residue comprising an amino acid side chain that comprises a carboxyl or sulfonic acid group;

Xₐₐ⁴ is an amino acid residue selected from Gly, Ala, Aib, and β-Ala;

Xₐₐ⁵ is an optionally substituted amino acid selected from Thr, Ser, Ala, Aib, Val, α-MeSer, α-MeThr, and α-MeVal;

Xₐₐ⁶ is an optionally substituted amino acid residue that is disubstituted at the α carbon, provided that one of the substituents is an optionally substituted aryl or heteroaryl;

Xₐₐ⁷ is an optionally substituted amino acid residue comprising an amino acid side chain that comprises a hydroxyl;

Xₐₐ⁸ is an optionally substituted amino acid residue selected from Ser, His, and Asn;

Xₐₐ⁹ is an optionally substituted amino acid residue comprising an amino acid side chain that comprises a carboxyl or sulfonic acid group;

Xₐₐ¹⁰ is an optionally substituted amino acid residue comprising an amino acid side chain that comprises a sulfide and/or an optionally substituted aryl or heteroaryl;

Xₐₐ¹¹ is an optionally substituted amino acid residue comprising an amino acid side chain that comprises a sulfide and/or an optionally substituted aryl or heteroaryl; and

R_{YC} is the C-terminal group of Xₐₐ¹¹ having the structure —C(O)N(R_{Y})₂, wherein R_{Y}, independently for each occurrence, is hydrogen or a PK modifier group.

### Pharmaceutical Compositions, Routes of Administration, and Dosing

In certain embodiments, the invention is directed to a pharmaceutical composition, comprising a compound, i.e. polypeptide, of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of, e.g., diabetes.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (m), most preferably 0.5 to 5 m, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above

### Combinations

The present invention includes within its scope pharmaceutical compositions comprising, as an active ingredient, a therapeutically effective amount of at least one of the polypeptides of sequence I, alone or in combination with a pharmaceutical carrier or diluent. Optionally, polypeptides of the present invention can be used in any one of the disclosed methods alone, in combination with other compounds of the invention, or in combination with one or more other therapeutic agent(s) as disclosed herein, e.g., an antidiabetic agent or other pharmaceutically active material.

The polypeptides of the present invention may be employed in combination with other GLP-1 receptor modulators (e.g., agonists or partial agonists, such as a peptide agonist) or other suitable therapeutic agents useful in the treatment of the aforementioned disorders including: anti-diabetic agents; anti-hyperglycemic agents; hypolipidemic/lipid lowering agents; anti-obesity agents (including appetite suppressants/modulators) and anti-hypertensive agents. In addition, the compounds of the present invention may be combined with one or more of the following therapeutic agents; infertility agents, agents for treating polycystic ovary syndrome, agents for treating growth disorders, agents for treating frailty, agents for treating arthritis, agents for preventing allograft rejection in transplantation, agents for treating autoimmune diseases, anti-AIDS agents, anti-osteoporosis agents, agents for treating immunomodulatory diseases, antithrombotic agents, agents for the treatment of cardiovascular disease, antibiotic agents, anti-psychotic agents, agents for treating chronic inflammatory bowel disease or syndrome and/or agents for treating anorexia nervosa.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), DPP-IV inhibitors, and SGLT2 inhibitors. Other suitable thiazolidinediones include Mitsubishi's MCC-555 (disclosed in U.S. Pat. No. 5,594,016), Glaxo-Welcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi).

Suitable PPAR alpha/gamma dual agonists include muraglitazar (Bristol-Myers Squibb), AR-HO39242 (Astra/Zeneca), GW-409544 (Glaxo-Wellcome), KRP297 (Kyorin Merck) as well as those disclosed by Murakami et al, “A Novel Insulin Sensitizer Acts as a Coligand for Peroxisome Proliferation-Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998), and in U.S. application Ser. No. 09/644,598, filed Sep. 18, 2000, the disclosure of which is incorporated herein by reference, employing dosages as set out therein, which compounds designated as preferred are preferred for use herein.

Suitable aP2 inhibitors include those disclosed in U.S. application Ser. No. 09/391,053, filed Sep. 7, 1999, and in U.S. application Ser. No. 09/519,079, filed Mar. 6, 2000, employing dosages as set out herein.

Suitable DPP4 inhibitors that may be used in combination with the compounds of the invention include those disclosed in WO 99/38501, WO 99/46272, WO 99/67279 (PROBIODRUG), WO 99/67278 (PROBIODRUG), WO 99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al, Biochemistry, 38(36), 11597-11603, 1999, LAF237, saxagliptin, MK0431, TSL-225 (tryptophyl-1,2,3,4-tetrahydroisoquinoline-3-carboxylic acid (disclosed by Yamada et al, Bioorg. & Med. Chem. Lett. 8 (1998) 1537-1540, 2-cyanopyrrolidides and 4-cyanopyrrolidides, as disclosed by Ashworth et al, Bioorg. & Med. Chem. Lett., Vol. 6, No. 22, pp 1163-1166 and 2745-2748 (1996) employing dosages as set out in the above references.

Suitable meglitinides include nateglinide (Novartis) or KAD1229 (PF/Kissei). Examples of other suitable glucagon-like peptide-1 (GLP-1) compounds that may be used in combination with the GLP-1 receptor modulators (e.g., agonists or partial agonists) of the present invention include GLP-1 (1-36) amide, GLP-1 (7-36) amide, GLP-1 (7-37) (as disclosed in U.S. Pat. No. 5,614,492 to Habener), as well as AC2993 (Amylin), LY-315902 (Lilly) and NN2211 (Novo Nordisk).

Examples of suitable hypolipidemic/lipid lowering agents for use in combination with the compounds of the present invention include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na+/bile acid cotransporter inhibitors, upregulators of LDL receptor activity, bile acid sequestrants, cholesterol ester transfer protein inhibitors (e.g., CP-529414 (Pfizer)) and/or nicotinic acid and derivatives thereof.

MTP inhibitors which may be employed as described above include those disclosed in U.S. Pat. Nos. 5,595,872, 5,739,135, 5,712,279, 5,760,246, 5,827,875, 5,885,983 and 5,962,440, all of which are incorporated by reference herein.

The HMG CoA reductase inhibitors which may be employed in combination with one or more compounds of Formula I include mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, visastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0142146 A2, and quinoline and pyridine derivatives, as disclosed in U.S. Pat. Nos. 5,506,219 and 5,691,322. Desired hypolipidemic agents are pravastatin, lovastatin, simvastatin, atorvastatin, fluvastatin, cerivastatin, atavastatin and ZD-4522.

In addition, phosphinic acid compounds useful in inhibiting HMG CoA reductase, such as those disclosed in GB 2205837, are suitable for use in combination with the compounds of the present invention.

The squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinyl-methyl)phosphonates, as well as other known squalene synthetase inhibitors, for example, as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J. A. C. S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, Summary.

The fibric acid derivatives which may be employed in combination with one or more compounds of Formula I include fenofibrate, gemfibrozil, clofibrate, bezafibrate, ciprofibrate, clinofibrate and the like, probucol, and related compounds, as disclosed in U.S. Pat. No. 3,674,836, probucol and gemfibrozil being preferred, bile acid sequestrants, such as cholestyramine, colestipol and DEAE-Sephadex (Secholex®, Policexide®), as well as lipostabil (Rhone-Poulenc), Eisai E-₅₀₅₀ (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents.

The ACAT inhibitor which may be employed in combination with one or more compounds of Formula I include those disclosed in Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd). The hypolipidemic agent may be an upregulator of LD2 receptor activity, such as MD-700 (Taisho Pharmaceutical Co. Ltd) and LY295427 (Eli Lilly).

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na+/bile acid cotransporter inhibitors for use in combination with the compounds of the invention include compounds as disclosed in Drugs of the Future, 24, 425-430 (1999).

The lipoxygenase inhibitors which may be employed in combination with one or more compounds of Formula I include 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-obesity agents for use in combination with the compounds of the present invention include a NPY receptor antagonist, a NPY—Y2 or NPY—Y4 receptor agonist, a MCH antagonist, a GHSR antagonist, a CRH antagonist, a beta 3 adrenergic agonist, a lipase inhibitor, a serotonin (and dopamine) reuptake inhibitor, a thyroid receptor beta drug, a CB-1 antagonist and/or an anorectic agent.

The beta 3 adrenergic agonists which may be optionally employed in combination with compounds of the present invention include AJ9677 (Takeda/Dainippon), L750355 (Merck), or CP331648 (Pfizer) or other known beta 3 agonists, as disclosed in U.S. Pat. Nos. 5,541,204, 5,770,615, 5,491,134, 5,776,983 and 5,488,064, with AJ9677, L750,355 and CP331648 being preferred.

Examples of lipase inhibitors which may be optionally employed in combination with compounds of the present invention include orlistat or ATL-962 (Alizyme), with orlistat being preferred.

The serotonin (and dopamine) reuptake inhibitor which may be optionally employed in combination with a compound of Formula I may be sibutramine, topiramate (Johnson & Johnson) or axokine (Regeneron), with sibutramine and topiramate being preferred. Examples of thyroid receptor beta compounds which may be optionally employed in combination with compounds of the present invention include thyroid receptor ligands, such as those disclosed in WO97/21993 (U. Cal SF), WO99/00353 (KaroBio) and GB98/284425 (KaroBio), with compounds of the KaroBio applications being preferred.

Examples of CB-1 antagonists which may be optionally employed in combination with compounds of the present invention include CB-1 antagonists and rimonabant (SR141716A) Examples of NPY—Y2 and NPY—Y4 receptor agonists include PYY(3-36) and Pancreatic Polypeptide (PP), respectively.

The anorectic agent which may be optionally employed in combination with compounds of the present invention include dexamphetamine, phentermine, phenylpropanolamine or mazindol, with dexamphetamine being preferred.

Examples of suitable anti-psychotic agents include clozapine, haloperidol, olanzapine (Zyprexa®), Prozac® and aripiprazole (Abilify®).

The aforementioned patents and patent applications are incorporated herein by reference. The above other therapeutic agents, when employed in combination with the compounds of the present invention may be used, for example, in those amounts indicated in the Physician's Desk Reference, as in the patents set out above or as otherwise determined by one of ordinary skill in the art.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Examples

The invention is further described in the following examples, which do not limit the scope of the invention described in the claims.

### Example 1: Framework of Peptide Design

The structure-based design and structure-activity analysis of a series of α-helical biased peptide mimics of the N-terminus of GLP-1 (Tables 1, 2 and 3) provided a framework to inform optimization by iterative chemical modifications to advance novel peptide analogs having drug-like properties, including high potency, metabolic stability and improved pharmacokinetics by parenteral or oral drug delivery.

The simplification of GLP-1 to a N-terminal fragment (i.e. His¹-Ala²-Glu³-Gly⁴-Thr⁵-Phe⁶-Thr⁷-Ser⁸-Asp⁹-Val¹⁰-Ser¹¹˜) consisting of eleven amino acids with key modifications, including Aib² (replacing Ala), α-MePhe(2,6-F)⁶ (replacing Phe), Bip¹⁰ (replacing Val) and Bip¹¹ (replacing Ser) has been reported previously (21-23). To inform novel N-terminal GLP-1 fragment analog optimization, twenty-one analogs of the generic structure H₂N-His-Aib-Glu-Gly-Thr-Xaa6-Thr-Ser-Asp-Val-Ser-C(O)NH₂ were synthesized and test for their GLP-1 receptor functional activity (EC₅₀, cAMP assay vide infra). As shown in Table 1, significantly increased potency was observed for Phe(2-F), Phe(2.6-F), Phe(2,3,4,5,6-F), (α-MePhe and α-MePhe(₂-F). These data confirmed what was previously described and expands the known structure-activity relationships of numerous other Phe analogs having modifications of the sidechain by varying substituents as well as homologation (to Hph), chiral inversion (to D-Phe), removal of the phenyl ring (to Ala) or replacement (to Bip or Aib). It was observed that both Phe⁶ and Ala⁶ were markedly less potent than their α-Me modified amino acid analogs (i.e., α-MePhe and Aib, respectively). Such data informs the impact of helical induction by oa-methylation that may be achieved within the core N-terminal eleven amino acid sequence of GLP-1.

TABLE 1
SAR of GLP₁₋₁₁ analogs with focus on Phe⁶ modifications

| RX- | | | | cAMP EC50, nM |
|---|---|---|---|---|
| Peptide | Peptide Structure (N-Terminus, Amino Acid Sequence, C-Terminus) | | | (0.1% albumin) |
| GLP-1 | H₂N | His¹-Ala-Glu-Gly-Thr-Phe⁶-Thr-Ser-Asp-Val-Ser¹¹ | Residues 12-30 | *** |
| 1-1 | H₂N | His-Aib-Glu-Gly-Thr-Phe-Thr-Ser-Asp-Bip-Bip | C(O)NH₂ | 6.82 |
| | | | | (San Diego) |
| 3-1 | H₂N | His-Aib-Glu-Gly-Thr-Phe(2-F)-Thr-Ser-Asp-Bip-Bip | C(O)NH₂ | 0.32 |
| 3-2 | H₂N | His-Aib-Glu-Gly-Thr-Phe(2-Cl)-Thr-Ser-Asp-Bip-Bip | C(O)NH₂ | 4.5 |
| 3-3 | H₂N | His-Aib-Glu-Gly-Thr-Phe(2-CF₃)-Thr-Ser-Asp-Bip-Bip | C(O)NH₂ | 97.1 |
| 3-4 | H₂N | His-Aib-Glu-Gly-Thr-Phe(2-CH₃)-Thr-Ser-Asp-Bip-Bip | C(O)NH₂ | 9.55 |
| 3-5 | H₂N | His-Aib-Glu-Gly-Thr-Phe(2-NO2)-Thr-Ser-Asp-Bip-Bip | C(O)NH₂ | 6.67 |
| 3-6 | H₂N | His-Aib-Glu-Gly-Thr-Phe(2-CN)-Thr-Ser-Asp-Bip-Bip | C(O)NH₂ | 33.1 |
| 3-7 | H₂N | His-Aib-Glu-Gly-Thr-Phe(3-CF₃)-Thr-Ser-Asp-Bip-Bip | C(O)NH₂ | 97.5 |
| 3-8 | H₂N | His-Aib-Glu-Gly-Thr-Phe(2,6F)-Thr-Ser-Asp-Bip-Bip | C(O)NH₂ | 0.16 |
| | | | | (0% albumin) |
| 3-9 | H₂N | His-Aib-Glu-Gly-Thr-Phe(3,4,5-F)-Thr-Ser-Asp-Bip- | C(O)NH₂ | 44.6 |
| | | Bip | | |
| 3-10 | H₂N | His-Aib-Glu-Gly-Thr-Phe(2,3,4,5,6-F)-Thr-Ser-Asp- | C(O)NH₂ | 1.8 |
| | | Bip-Bip | | |
| 3-11 | H₂N | His-Aib-Glu-Gly-Thr-hPhe-Thr-Ser-Asp-Bip-Bip | C(O)NH₂ | 32.6 |
| 3-12 | H₂N | His-Aib-Glu-Gly-Thr-Tyr-Thr-Ser-Asp-Bip-Bip | C(O)NH₂ | 7.3 |
| 3-13 | H₂N | His-Aib-Glu-Gly-Thr-D-Phe-Thr-Ser-Asp-Bip-Bip | C(O)NH₂ | 57.0 |
| 3-14 | H₂N | His-Aib-Glu-Gly-Thr-Trp-Thr-Ser-Asp-Bip-Bip | C(O)NH₂ | 67.6 |
| 3-15 | H₂N | His-Aib-Glu-Gly-Thr-Bip-Thr-Ser-Asp-Bip-Bip | C(O)NH₂ | 26.2 |
| 1-2 | H₂N | His-Aib-Glu-Gly-Thr-α-MePhe(2-F)-Thr-Ser-Asp-Bip- | C(O)NH₂ | 0.09 |
| | | Bip | | |
| 3-16 | H₂N | His-Aib-Glu-Gly-Thr-α-MePhe-Thr-Ser-Asp-Bip-Bip | C(O)NH₂ | 0.32 |
| 3-17 | H₂N | His-Aib-Glu-Gly-Thr-α-MePhe-Thr-Ser-Asp-Bip-Bip | C(O)NH₂ | 0.08 |
| | | | | (0% albumin) |
| 3-18 | H₂N | His-Aib-Glu-Gly-Thr-Aib-Thr-Ser-Asp-Bip-Bip | C(O)NH₂ | 152 |
| 3-19 | H₂N | His-Aib-Glu-Gly-Thr-Ala-Thr-Ser-Asp-Bip-Bip | C(O)NH₂ | 2,800 |
| | | | | (0% albumin) |

For the first time relative to this series of N-terminal GLP-1 fragment analogs, Ala-scanning was performed (Table 2) to inform novel GLP-1 analog optimization. The structure-activity relationship of ten peptides showed Ala substitution for His¹, Gly⁴ and Thr⁷ resulted in >100-fold decreased potency, whereas Aib², Glu³, Thr⁵, Ser⁸, and Asp⁹ substitutions by Ala resulted in 100-fold decreased potency. Most noteworthy was the >1000-fold decreased potency shown by Ala substitutions of Bip¹⁰ and Bip¹¹. Such data informs the impact of simplification of amino acid sidechains to a methyl group (Ala) and that several amino acids (e.g., Glu⁵, Thr⁵, Ser⁸, and Asp⁹ may tolerate further modifications to modulate their hydrophilic character (e.g., H-bonding and charge) and helicity propensity (vide infra; Aib-scanning) to enable optimization of the drug-like properties within Formula I.

TABLE 2
SAR of GLP1-11 analogs with singular Ala-scanning of 3-1

| RX- | | | | cAMP EC50, nM |
|---|---|---|---|---|
| Peptide | Peptide Structure (N-Terminus, Amino Acid Sequence, C-Terminus) | | | (0.1% albumin) |
| GLP-1 | H₂N | His¹-Ala-Glu-Gly-Thr-Phe⁶-Thr-Ser-Asp-Val-Ser¹¹ | Residues | *** |
| | | | 12-30 | |
| 3-1 | H₂N | His-Aib-Glu-Gly-Thr-Phe(2-F)-Thr-Ser-Asp-Bip-Bip | C(O)NH₂ | 0.32 |
| 3-20 | H₂N | Ala-Aib-Glu-Gly-Thr-Phe(2-F)-Thr-Ser-Asp-Bip-Bip | C(O)NH₂ | 66.0 |
| 3-21 | H₂N | His-Ala-Glu-Gly-Thr-Phe(2-F)-Thr-Ser-Asp-Bip-Bip | C(O)NH₂ | 0.29 |
| 3-22 | H₂N | His-Aib-Ala-Gly-Thr-Phe(2-F)-Thr-Ser-Asp-Bip-Bip | C(O)NH₂ | 20.3 |
| 3-23 | H₂N | His-Aib-Glu-Ala-Thr-Phe(2F)-Thr-Ser-Asp-Bip-Bip | C(O)NH₂ | 347 |
| | | | | (0% albumin) |
| 3-24 | H₂N | His-Aib-Glu-Gly-Ala-Phe(2-F)-Thr-Ser-Asp-Bip-Bip | C(O)NH₂ | 28.1 |
| 3-25 | H₂N | His-Aib-Glu-Gly-Thr-Phe(2-F)-Ala-Ser-Asp-Bip-Bip | C(O)NH₂ | 79.1 |
| 3-26 | H₂N | His-Aib-Glu-Gly-Thr-Phe(2-F)-Thr-Ala-Asp-Bip-Bip | C(O)NH₂ | 18.9 |
| 3-27 | H₂N | His-Aib-Glu-Gly-Thr-Phe(2-F)-Thr-Ser-Ala-Bip-Bip | C(O)NH₂ | 67.0 |
| 3-28 | H₂N | His-Aib-Glu-Gly-Thr-Phe(2-F)-Thr-Ser-Asp-Ala-Bip | C(O)NH₂ | >1000 |
| 3-29 | H₂N | His-Aib-Glu-Gly-Thr-Phe(2-F)-Thr-Ser-Asp-Bip-Ala | C(O)NH₂ | >1000 |

Also, for the first time relative to this series of N-terminal GLP-1 fragment analogs, Aib-scanning was performed (Table 3) to inform novel GLP-1 analog optimization. The structure-activity relationships of eight peptides showed Aib substitution for Ser⁴, Thr⁷, Ser⁸, and Asp9 resulted in >1000-fold decreased potency, whereas His¹, Glu³, Gly⁴, and Thr⁵ substitutions by Aib resulted in <200-fold decreased potency. In fact, His¹ replacement by Aib was surprisingly potent (only <30-fold difference). Such data informs the design of both specific modifications by α-methylation or in some cases (e.g., His¹, Glu³ or Thr⁵) replacement by Aib. Furthermore, such data implicates α-methylation to nucleate and/or sustain helicity which is known by X-ray structures of GLP-1 and N-terminal fragment analogs (vide infra), and that incorporating such α-methylation may enable optimization of the drug-like properties within Formula I.

TABLE 3
SAR of GLP₁₋₁₁ analogs with Aib-scanning of 3-1

| | | | | cAMP |
|---|---|---|---|---|
| | | | | EC50, nM |
| RX- | | | | (0.1% |
| Peptide | Peptide Structure (N-Terminus, Amino Acid Sequence, C-Terminus) | | | albumin) |
| GLP-1 | H | His¹-Ala-Glu-Gly-Thr-Phe⁶-Thr-Ser-Asp-Val-Ser¹¹ | Residues | *** |
| | | | 12-30 | |
| 1-2 | H₂N | His-Aib-Glu-Gly-Thr-α-MePhe(2-F)-Thr-Ser-Asp-Bip-Bip | C(O)NH₂ | 0.09 |
| 3-30 | H₂N | Aib-Aib-Glu-Gly-Thr-αMePhe(2-F)-Thr-Ser-Asp-Bip-Bip | C(O)NH₂ | 2.7 |
| 3-31 | H₂N | His-Aib-Aib-Gly-Thr-αMePhe(2-F)-Thr-Ser-Asp-Bip-Bip | C(O)NH₂ | 4.9 |
| 3-32 | H₂N | His-Aib-Glu-Aib-Thr-αMePhe(2-F)-Thr-Ser-Asp-Bip-Bip | C(O)NH₂ | 15.8 |
| 3-33 | H₂N | His-Aib-Glu-Gly-Aib-αMePhe(2-F)-Thr-Ser-Asp-Bip-Bip | C(O)NH₂ | 7.3 |
| 3-34 | H₂N | His-Aib-Glu-Gly-Thr-Aib-Thr-Ser-Asp-Bip-Bip | C(O)NH₂ | 152 |
| 3-35 | H₂N | His-Aib-Glu-Gly-Thr-αMePhe(2-F)-Aib-Ser-Asp-Bip-Bip | C(O)NH₂ | 91.4 |
| 3-36 | H₂N | His-Aib-Glu-Gly-Thr-αMePhe(2-F)-Thr-Aib-Asp-Bip-Bip | C(O)NH₂ | 150 |
| 3-37 | H₂N | His-Aib-Glu-Gly-Thr-αMePhe(2-F)-Thr-Ser-Aib-Bip-Bip | C(O)NH₂ | 91.6 |

### Example 2: GLP-1 Peptide Agonists that Incorporate Chemical Modifications to Achieve Drug-Like Properties

Exemplified in Tables 4, 5 and 6 are novel N-terminal GLP-1 peptides representative of the scope of Formula 1 and illustrating the design and structure-activity properties of three series of analogs having N-terminal and/or C-terminal modifications. Specifically, peptides having N-terminal modifications which include extension beyond His¹ (e.g., 2-1, 2-2, 2-3, 2-4, and 2-5) were designed from computational modeling studies (vide infra) and predicted to bind to the GLP-1 receptor (Table 4). These exemplary peptides showed GLP-1 receptor agonist functional potencies within 3-fold of the parent peptide analog (1-1). Furthermore, peptides having both replacement of His¹ and Glu³ as well as extension beyond His¹ (e.g., 2-6, 2-7, 2-8, and 2-9) were designed from computer modeling studies (vide infra) and predicted to bind to the GLP-1 receptor (Table 1). These exemplary peptides showed GLP-1 receptor agonist functional potencies within 100-fold of the parent peptide analog (1-2). Additionally, peptides having replacement of Aib2 (3-38, 3-39, and 3-40) showed (Table 4) similar potency (e.g., Pro²), slightly less potency (Val²) or significantly greater potency (α-MePro²) than the parent peptide analog (1-2). Moreover, a series of exemplary peptides incorporating combinations of the preceding N-terminal modifications are enumerated (Table 4). Collectively, such described N-terminal modifications will enable selection of GLP-1 peptide analogs having superior drug-like properties relative to agonist potency, metabolic stability, GLP-1 receptor (and GLP-1 receptor family) selectivity (and co-selectivity), and biophysical properties (e.g., helicity, solubility and hydrophobicity/hydrophilicity).

TABLE 4
SAR of GLP₁₋₁₁ analogs with N-terminal modifications of 1-1 & 3-1

| | | | | cAMP EC50, |
|---|---|---|---|---|
| RX- | | | | nM (0.1% |
| Peptide | Peptide Structure (N-Terminus, Amino Acid Sequence, C-Terminus) | | | albumin) |
| GLP-1 | H | His¹-Ala-Glu-Gly-Thr-Phe⁶-Thr-Ser-Asp-Val-Ser¹¹ | Residues | *** |
| | | | 12-30 | |

| | | | | |
|---|---|---|---|---|
| 1-1 | H₂N | His-Aib-Glu-Gly-Thr-Phe-Thr-Ser-Asp-Bip-Bip | C(O)NH₂ | 6.82 |
| 2-1 | H₂N | Arg-His-Aib-Glu-Gly-Thr-Phe-Thr-Ser-Asp-Bip-Bip | C(O)NH₂ | 10.6 |
| 2-2 | H₂N | Glu-His-Aib-Glu-Gly-Thr-Phe-Thr-Ser-Asp-Bip-Bip | C(O)NH₂ | 40.1 |
| 2-3 | H₂N | Gly-His-Aib-Glu-Gly-Thr-Phe-Thr-Ser-Asp-Bip-Bip | C(O)NH₂ | 21.1 |
| 2-4 | H₂N | Pro-His-Aib-Glu-Gly-Thr-Phe-Thr-Ser-Asp-Bip-Bip | C(O)NH₂ | 14.7 |
| 2-5 | H₂N | Trp-His-Aib-Glu-Gly-Thr-Phe-Thr-Ser-Asp-Bip-Bip | C(O)NH₂ | 10.9 |
| 1-2 | H₂N | His-Aib-Glu-Gly-Thr-α-MePhe(2-F)-Thr-Ser-Asp-Bip-Bip | C(O)NH₂ | 0.09 |
| 2-6 | H₂N | Glu-Leu-Aib-Glu-Gly-Thr-α-MePhe(2-F)-Thr-Ser-Asp-Bip-Bip | C(O)NH₂ | 9.2 |
| 2-7 | H₂N | Glu-Leu-Aib-Asp-Gly-Thr-α-MePhe(2-F)-Thr-Ser-Asp-Bip-Bip | C(O)NH₂ | 3.1 |
| 2-8 | H₂N | Phe-Leu-Aib-Glu-Gly-Thr-α-MePhe(2-F)-Thr-Ser-Asp-Bip-Bip | C(O)NH₂ | 4.1 |
| 2-9 | H₂N | His-Leu-Aib-Asp-Gly-Thr-α-MePhe(2-F)-Thr-Ser-Asp-Bip-Bip | C(O)NH₂ | 6.4 |
| 3-38 | H₂N | His-Pro-Glu-Gly-Thr-α-MePhe(2-F)-Thr-Ser-Asp-Bip-Bip | C(O)NH₂ | 0.04 |
| 3-39 | H₂N | His-Val-Glu-Gly-Thr-α-MePhe(2-F)-Thr-Ser-Asp-Bip-Bip | C(O)NH₂ | 0.37 |
| 3-40 | H₂N | His-α-MePro-Glu-Gly-Thr-α-MePhe(2-F)-Thr-Ser-Asp-Bip-Bip | C(O)NH₂ | 0.003 |
| 3-41 | H₂N | His-α-MePro-Glu-Gly-Thr-α-MePhe-Thr-Ser-Asp-Bip-Bip | C(O)NH₂ | — |
| 3-42 | H₂N | Arg-His-α-MePro-Glu-Gly-Thr-α-MePhe-Thr-Ser-Asp-Bip-Bip | C(O)NH₂ | — |
| 3-43 | H₂N | Glu-His-α-MePro-Glu-Gly-Thr-α-MePhe-Thr-Ser-Asp-Bip-Bip | C(O)NH₂ | — |
| 3-44 | H₂N | Gly-His-α-MePro-Glu-Gly-Thr-α-MePhe-Thr-Ser-Asp-Bip-Bip | C(O)NH₂ | — |
| 3-45 | H₂N | Pro-His-α-MePro-Glu-Gly-Thr-α-MePhe-Thr-Ser-Asp-Bip-Bip | C(O)NH₂ | — |
| 3-46 | H₂N | Trp-His-α-MePro-Glu-Gly-Thr-α-MePhe-Thr-Ser-Asp-Bip-Bip | C(O)NH₂ | — |
| 3-47 | H₂N | Arg-His-Pro-Glu-Gly-Thr-α-MePhe-Thr-Ser-Asp-Bip-Bip | C(O)NH₂ | — |
| 3-48 | H₂N | Glu-His-Pro-Glu-Gly-Thr-α-MePhe-Thr-Ser-Asp-Bip-Bip | C(O)NH₂ | — |
| 3-59 | H₂N | Gly-His-Pro-Glu-Gly-Thr-α-MePhe-Thr-Ser-Asp-Bip-Bip | C(O)NH₂ | — |
| 3-50 | H₂N | Pro-His-Pro-Glu-Gly-Thr-α-MePhe-Thr-Ser-Asp-Bip-Bip | C(O)NH₂ | — |
| 3-51 | H₂N | Trp-His-Pro-Glu-Gly-Thr-α-MePhe-Thr-Ser-Asp-Bip-Bip | C(O)NH₂ | — |
| 3-52 | H₂N | Aib-α-MePro-Glu-Gly-Thr-α-MePhe-Thr-Ser-Asp-Bip-Bip | C(O)NH₂ | — |
| 3-53 | H₂N | His-α-MePro-Aib-Gly-Thr-α-MePhe-Thr-Ser-Asp-Bip-Bip | C(O)NH₂ | — |
| 3-54 | H₂N | Aib-Pro-Glu-Gly-Thr-α-MePhe-Thr-Ser-Asp-Bip-Bip | C(O)NH₂ | — |
| 3-55 | H₂N | His-Pro-Aib-Gly-Thr-α-MePhe-Thr-Ser-Asp-Bip-Bip | C(O)NH₂ | — |
| 3-56 | H₂N | Aib-Pro-Glu-Gly-Thr-α-MePhe(2-F)-Thr-Ser-Asp-Bip-Bip | C(O)NH₂ | — |
| 3-57 | H₂N | His-Pro-Aib-Gly-Thr-α-MePhe(—2-F)-Thr-Ser-Asp-Bip-Bip | C(O)NH₂ | — |
| 3-58 | H₂N | His-α-MePro-Glu-Gly-Thr-α-MePhe(2-F)-Thr-Ser-Asp-Bip-Bip-Aib | C(O)AIB(NH₂) | 0.3 |
| 3-59 | H₂N | His-α-MePro-Glu-Gly-Thr-α-MePhe(2-F)-α-MeSer-Ser-Asp-Bip-Bip | C(O)NH₂ | 20.9 |
| 3-60 | H₂N | His-α-MePro-Glu-Gly-Thr-α-MePhe(2-F)-Thr-Ser-Asp-Aib-Bip | C(O)NH₂ | >1000 |
| 3-61 | H₂N | His-α-MePro-Glu-Gly-Thr-α-MePhe-Thr-Ser-Asp-Bip-Aib | C(O)NH₂ | 8.3 |
| 3-62 | H₂N | His-Aib-Glu-Gly-Thr-α-MePhe(2-F)-Asn-Ser-Asp-Bip-Bip | C(O)NH₂ | 3.1 |

Specifically, peptides incorporating C-terminal modifications of Bip¹⁰-Bip¹¹ such as, but not limited to, Hph¹⁰-Bip¹¹, Bip¹⁰-Hph¹¹, Bip¹⁰-α-MeHph¹¹, α-MeHph¹⁰-Bip¹¹, Bip¹⁰-Hph(4-OH)¹¹ and Bip¹⁰-NH—(CH₂)₃-phenyl (see e.g., 1-3, 1-4, 4-1, 4-2, 4-3, 4-4, 4-4a) were designed as GLP-1 receptor agonists (Table 5). These exemplary peptides showed GLP-1 receptor agonist functional potencies in the range of 30-to 600-fold of the parent peptide (3-1). C-terminal replacement of the carboxamide by a carboxylic acid (4-1) or by a hydrogen as each were less potent (about 30- or >1,000-fold, respectively) to their parent peptide analogs (1-4 and 3-1, respectively). Furthermore, peptides having novel modified Cys¹⁰ or Cys¹¹ or α-MeCys analogs thereof may be synthetically converted to thioether within the scope of varying S-aryl, S-heteroaryl, S-heterocyclyl, and S-cycloalkyl groups (e.g., 4-5 to 4-36, Table 5). Moreover, a series of exemplary peptides incorporating combinations of the preceding N-terminal modifications are enumerated (Table 5). Collectively, such described N-terminal modifications will enable selection of GLP-1 peptide analogs having superior drug-like properties relative to agonist potency, metabolic stability, GLP-1 receptor (and GLP-1 receptor family) selectivity (and co-selectivity), and biophysical properties (e.g., helicity, solubility and hydrophobicity/hydrophilicity).

TABLE 5
SAR of GLP₁₋₁₁ analogs with C-terminal modifications of 1-1 & 3-1

| | | | | cAMP |
|---|---|---|---|---|
| | | | | EC50, nM |
| RX- | | | | (0% |
| Peptide | Peptide Structure (N-Terminus, Amino Acid Sequence, C-Terminus) | | | albumin) |
| GLP-1 | H₂N | His¹-Ala-Glu-Gly-Thr-Phe⁶-Thr-Ser-Asp-Val-Ser¹¹ | Residues | *** |
| | | | 12-30 | |
| 1-1 | H₂N | His-Aib-Glu-Gly-Thr-Phe-Thr-Ser-Asp-Bip-Bip | C(O)NH₂ | 6.82 |
| 1-2 | H₂N | His-Aib-Glu-Gly-Thr-α-MePhe(2-F)-Thr-Ser-Asp-Bip-Bip | C(O)NH₂ | 0.09 |
| 1-3 | H₂N | His-Aib-Glu-Gly-Thr-α-MePhe(2-F)-Thr-Ser-Asp-Hph-Bip | C(O)NH₂ | 11 |
| 1-4 | H₂N | His-Aib-Glu-Gly-Thr-α-MePhe(2-F)-Thr-Ser-Asp-Bip-Hph | C(O)NH₂ | 11 |
| 4-1 | H₂N | His-Aib-Glu-Gly-Thr-α-MePhe(2-F)-Thr-Ser-Asp-Bip-Hph | C(O)OH | 272 |
| 4-2 | H₂N | His-Aib-Glu-Gly-Thr-α-MePhe(2-F)-Thr-Ser-Asp-Bip-α-MeHph | C(O)NH₂ | 5.5 |
| 4-3 | H₂N | His-Aib-Glu-Gly-Thr-α-MePhe(2-F)-Thr-Ser-Asp-α-MeHph-Bip | C(O)NH₂ | 184 |
| 4-4 | H₂N | His-Aib-Glu-Gly-Thr-α-MePhe(2-F)-Thr-Ser-Asp-Bip-Hph(4-OH) | C(O)NH₂ | 16.6 |
| 4-5 | H₂N | His-Aib-Glu-Gly-Thr-α-MePhe(2-F)-Thr-Ser-Asp-Cys(S-Ary11)-Bip | C(O)NH₂ | — |
| 4-6 | H₂N | His-Aib-Glu-Gly-Thr-α-MePhe(2-F)-Thr-Ser-Asp-Cys(S-Ary12)-Bip | C(O)NH₂ | — |
| 4-7 | H₂N | His-Aib-Glu-Gly-Thr-α-MePhe(2-F)-Thr-Ser-Asp-Cys(S-Ary13)-Bip | C(O)NH₂ | — |
| 4-8 | H₂N | His-Aib-Glu-Gly-Thr-α-MePhe(2-F)-Thr-Ser-Asp-α-MeCys(S-Ary11)- | C(O)NH₂ | — |
| | | Bip | | |
| 4-9 | H₂N | His-Aib-Glu-Gly-Thr-α-MePhe(2-F)-Thr-Ser-Asp-α-MeCys(S-Ary12)- | C(O)NH₂ | — |
| | | Bip | | |
| 4-10 | H₂N | His-Aib-Glu-Gly-Thr-α-MePhe(2-F)-Thr-Ser-Asp-Bip-Cys(S-Ary11) | C(O)NH₂ | — |
| 4-11 | H₂N | His-Aib-Glu-Gly-Thr-α-MePhe(2-F)-Thr-Ser-Asp-Bip-Cys(S-Ary12) | C(O)NH₂ | — |
| 4-12 | H₂N | His-Aib-Glu-Gly-Thr-α-MePhe(2-F)-Thr-Ser-Asp-Bip-Cys(S-Ary13) | C(O)NH₂ | — |
| 4-13 | H₂N | His-Aib-Glu-Gly-Thr-α-MePhe(2-F)-Thr-Ser-Asp-Bip-α-MeCys(S- | C(O)NH₂ | — |
| | | Ary11) | | |
| 4-14 | H₂N | His-Aib-Glu-Gly-Thr-α-MePhe(2-F)-Thr-Ser-Asp-Bip-α-MeCys(S- | C(O)NH₂ | — |
| | | Ary12) | | |
| 4-15 | H₂N | His-Aib-Glu-Gly-Thr-α-MePhe(2-F)-Thr-Ser-Asp-Bip-α-MeCys(S- | C(O)NH₂ | — |
| | | Ary13) | | |
| 4-16 | H₂N | His-α-MePro-Glu-Gly-Thr-α-MePhe(2-F)-Thr-Ser-Asp-Bip-Cys(S- | C(O)NH₂ | — |
| | | Ary11) | | |
| 4-17 | H₂N | His-α-MePro-Glu-Gly-Thr-α-MePhe(2-F)-Thr-Ser-Asp-Bip-Cys(S- | C(O)NH₂ | — |
| | | Ary12) | | |
| 4-18 | H₂N | His-α-MePro-Glu-Gly-Thr-α-MePhe(2-F)-Thr-Ser-Asp-Bip-Cys(S- | C(O)NH₂ | — |
| | | Ary13) | | |
| 4-19 | H₂N | His-α-MePro-Glu-Gly-Thr-α-MePhe-Thr-Ser-Asp-Bip-α-MeCys(S- | C(O)NH₂ | — |
| | | Ary11) | | |
| 4-20 | H₂N | His-α-MePro-Glu-Gly-Thr-α-MePhe-Thr-Ser-Asp-Bip-α-MeCys(S- | C(O)NH₂ | — |
| | | Ary12) | | |
| 4-21 | H₂N | His-α-MePro-Glu-Gly-Thr-α-MePhe-Thr-Ser-Asp-Bip-α-MeCys(S- | C(O)NH₂ | — |
| | | Ary13) | | |
| 4-22 | H₂N | His-Pro-Glu-Gly-Thr-α-MePhe-Thr-Ser-Asp-Bip-α-MeCys(S-Ary11) | C(O)NH₂ | — |
| 4-23 | H₂N | His-Pro-Glu-Gly-Thr-α-MePhe-Thr-Ser-Asp-Bip-α-MeCys(S-Ary12) | C(O)NH₂ | — |
| 4-24 | H₂N | His-Pro-Glu-Gly-Thr-α-MePhe-Thr-Ser-Asp-Bip-α-MeCys(S-Ary13) | C(O)NH₂ | — |
| 4-25 | H₂N | His-Aib-Glu-Gly-Thr-α-MePhe-Thr-Ser-Asp-Bip-α-MeCys(S-Ary11) | C(O)NH₂ | — |
| 4-26 | H₂N | His-Aib-Glu-Gly-Thr-α-MePhe-Thr-Ser-Asp-Bip-α-MeCys(S-Ary12) | C(O)NH₂ | — |
| 4-27 | H₂N | His-Aib-Glu-Gly-Thr-α-MePhe-Thr-Ser-Asp-Bip-α-MeCys(S-Ary13) | C(O)NH₂ | — |

Furthermore, peptides having C-terminal modifications which include extension beyond Bip¹¹ (e.g., 4-28, 4-29, 4-30, 4-31, and 4-32) were designed as novel N-terminal GLP-1 peptide analogs and intermediates with respect to further functionalization of Lys (albeit not limited to Lys as any amino acid having a primary amino group, such as Dap or Dab, would be as effective) and conjugation with PK-modifier groups (Table 6). These exemplary peptides showed GLP-1 receptor agonist functional potencies within a range of 20- to 100-fold of the parent peptide analog (3-1). Such peptides illustrate the design of C-terminally extended linkers, including, but not limited to, polyethylene glycol (e.g., AEEA) or simple amino acids (e.g., Gly or Aib) or combinations thereof to yet another chemical moiety hereinafter referred to as pharmacokinetic (PK) modifiers which exploit known binders to human serum albumin (HSA) such as, but not limited to fatty acids and aryl-halides (10-14). A series of peptide analogs of 1-4 (e.g., 4-35, 4-36, and 4-37) exemplify C-terminal backbone extension through the linker (AEEA)₂ and conjugation to PK modifiers (C18 diacid or 4-I-phenylproprionic acid) via Dap as shown in Table 6 and noting the designation of R_{Y1}, R_{Y2} and R_{Y3} (vide infra) with respect to the C-terminal modifications that incorporate the linker and PK modifier conjugates. The two peptides incorporating PK modifiers showed GLP-1 receptor agonist functional potencies in the range of equipotency (4-37) to 15-fold lower potency (4-36) of the parent peptide (1-4). C-terminal replacement of the carboxamide by a carboxylic acid (4-1) or by a hydrogen as each were less potent (about 30- or >1,000-fold, respectively) to their parent peptide analogs (1-4 and 3-1 respectively).

TABLE 6
SAR of GLP₁₋₁₁ analogs (C-terminal PK-modifier conjugates of 3-1 and 3-40)

| | | | | cAMP EC50, |
|---|---|---|---|---|
| | | | | nM |
| RX- | | | | (0.1% |
| Peptide | Peptide Structure (N-Terminus, Amino Acid Sequence, C-Terminus) | | | albumin) |
| GLP-1 | H | His¹-Ala-Glu-Gly-Thr-Phe⁶-Thr-Ser-Asp-Val-Ser¹¹ | Residues 12-30 | *** |
| 1-2 | H₂N | His-Aib-Glu-Gly-Thr-α-MePhe(2-F)-Thr-Ser-Asp-Bip-Bip | C(O)NH2 | 0.09 |
| 4-28 | H₂N | His-Aib-Glu-Gly-Thr-α-MePhe(2F)-Thr-Ser-Asp-Bip-Bip | Lys-NH2 | 16.1 |
| 4-29 | H₂N | His-Aib-Glu-Gly-Thr-α-MePhe(2F)-Thr-Ser-Asp-Bip-Bip | C(O)(AEEA)2- | 10.0 |
| | | | NH2 | |
| 4-30 | H₂N | His-Aib-Glu-Gly-Thr-α-MePhe(2F)-Thr-Ser-Asp-Bip-Bip | C(O)(AEEA)2- | 14.0 |
| | | | Lys-NH2 | |
| 4-31 | H₂N | His-Aib-Glu-Gly-Thr-α-MePhe(2F)-Thr-Ser-Asp-Bip-Bip | C(O)(AEEA)₃- | 31.2 |
| | | | Lys-NH2 | |
| 4-32 | H₂N | His-Aib-Glu-Gly-Thr-α-MePhe(2F)-Thr-Ser-Asp-Bip-Bip | (Gly)₃-Lys- | 7.7 |
| | | | NH2 | |
| 4-33 | H₂N | His-Aib-Glu-Gly-Thr-α-MePhe(2F)-Thr-Ser-Asp-Bip-Bip | Aib-(Gly)2- | 33.7 |
| | | | Lys-NH2 | |
| 4-34 | H₂N | His-Aib-Glu-Gly-Thr-α-MePhe(2-F)-Thr-Ser-Asp-Bip-Hph | C(O)NH₂ | 11 |
| 4-35 | H₂N | His-Aib-Glu-Gly-Thr-α-MePhe(2-F)-Thr-Ser-Asp-Bip-Hph | C(O)NH-R_{Y1} | 67.2 |
| 4-36 | H₂N | His-Aib-Glu-Gly-Thr-α-MePhe(2-F)-Thr-Ser-Asp-Bip-Hph | C(O)NH-R_{Y2} | 70.9 |
| 4-37 | H₂N | His-Aib-Glu-Gly-Thr-α-MePhe(2-F)-Thr-Ser-Asp-Bip-Hph | C(O)NH-R_{Y3} | 4.08 |
| 4-4 | H₂N | His-Aib-Glu-Gly-Thr-α-MePhe(2-F)-Thr-Ser-Asp-Bip-Hph(4- | C(O)NH₂ | 16.6 |
| | | OH) | | |
| 4-38 | H₂N | His-Aib-Glu-Gly-Thr-α-MePhe(2-F)-Thr-Ser-Asp-Bip-Hph(4- | C(O)NH₂ | 320 |
| | | OR4) | | |
| 4-39 | H₂N | His-Aib-Glu-Gly-Thr-α-MePhe(2-F)-Thr-Ser-Asp-Bip-Hph(4- | C(O)NH₂ | 232 |
| | | OR5) | | |
| 4-40 | H₂N | His-Aib-Glu-Gly-Thr-α-MePhe(2-F)-Thr-Ser-Asp-Bip-Hph(4- | C(O)NH₂ | 49.7 |
| | | OR6) | | |
| (D)-4-41 | H₂N | His-Aib-Glu-Gly-Thr-α-MePhe(2-F)-Thr-Ser-Asp-Bip-D-Tyr | C(O)NH₂ | 130 |
| (D)-4-42 | H₂N | His-Aib-Glu-Gly-Thr-α-MePhe(2-F)-Thr-Ser-Asp-Bip-D- | C(O)NH₂ | 791 |
| | | Tyr(O-R4) | | |
| (D)-4-43 | H₂N | His-Aib-Glu-Gly-Thr-α-MePhe(2-F)-Thr-Ser-Asp-Bip-D- | C(O)NH₂ | 693 |
| | | Tyr(O-R5) | | |
| (D)-4-44 | H₂N | His-Aib-Glu-Gly-Thr-α-MePhe(2-F)-Thr-Ser-Asp-Bip-D- | C(O)NH₂ | 299 |
| | | Tyr(O-R6) | | |
| 4-45 | H₂N | His-Aib-Glu-Gly-Thr-α-MePhe(2-F)-Thr-Ser-Asp-Bip-Bip | C(O)NH-R_{Y7} | 100.0 |
| 4-46 | H₂N | His-Aib-Glu-Gly-Thr-α-MePhe(2-F)-Thr-Ser-Asp-Bip-Bip | C(O)NH-R_{Y8} | 5.4 |
| 4-47 | H₂N | His-Aib-Glu-Gly-Thr-α-MePhe(2-F)-Thr-Ser-Asp-Bip-Bip | C(O)NH-R_{Y9} | 0.6 |
| 4-48 | H₂N | His-Aib-Glu-Gly-Thr-α-MePhe(2-F)-Thr-Ser-Asp-Bip-Bip | C(0)NH-R_{Y10} | 9.7 |
| 4-49 | H₂N | His-Aib-Glu-Gly-Thr-α-MePhe(2-F)-Thr-Ser-Asp-Bip-Bip | C(O)NH-R_{Y11} | 5.0 |
| 4-50 | H₂N | His-Aib-Glu-Gly-Thr-α-MePhe(2-F)-Thr-Ser-Asp-Bip-Bip | C(O)NH-R_{Y12} | 12.0 |
| 4-51 | H₂N | His-α-MePro-Glu-Gly-Thr-α-MePhe(2-F)-Thr-Ser-Asp-Bip- | C(O)NH-R_{Y9} | — |
| | | Bip | | |
| 4-52 | H₂N | His-α-MePro-Glu-Gly-Thr-α-MePhe(2-F)-Thr-Ser-Asp-Bip- | C(O)NH-R_{Y9} | — |
| | | Bip | | |
| 4-53 | H₂N | His-α-MePro-Glu-Gly-Thr-α-MePhe(2-F)-Thr-Ser-Asp-Bip- | C(O)NH-RY₉ | — |
| | | Bip | | |
| 4-54 | H₂N | His-α-MePro-Glu-Gly-Thr-α-MePhe(2-F)-Thr-Ser-Asp-Bip- | C(O)NH-R_{Y9} | — |
| | | Bip | | |
| 4-55 | H₂N | His-Aib-Glu-Gly-Thr-α-MePhe(2-F)-Thr-Ser-Asp-Phe(4-R10)- | C(O)NH₂ | — |
| | | Bip | | |
| 4-56 | H₂N | His-Aib-Glu-Gly-Thr-α-MePhe(2-F)-Thr-Ser-Asp-Bip-Phe(4- | C(O)NH₂ | — |
| | | R10) | | |
| 4-57 | H₂N | His-Aib-Glu-Gly-Thr-α-MePhe(2-F)-Thr-Ser-Asp-Bip-Bip | C(O)NH-R_{Y13} | 14.0 |
| 4-58 | H₂N | His-Aib-Glu-Gly-Thr-α-MePhe(2-F)-Thr-Ser-Asp-Bip-Bip | C(O)NH-R_{Y14} | 7.7 |
| 4-59 | H₂N | His-α-MePro -Glu-Gly-Thr-α-MePhe(2-F)-Thr-Ser-Asp-Bip- | C(O)NH-R_{Y15} | 1.3 |
| | | Bip | | |
| 4-60 | H₂N | His-α-MePro-Glu-Gly-Thr-α-MePhe(2-F)-Thr-Ser-Asp-Bip- | C(O)NH-R_{Y16} | 2.2 |
| | | Bip | | |
| 4-61 | H₂N | His-α-MePro-Glu-Gly-Thr-α-MePhe(2-F)-Thr-Ser-Asp-Bip- | C(O)NH-R_{Y17} | 272 |
| | | Bip | | |
| 4-62 | H₂N | His-α-MePro-Glu-Gly-Thr-α-MePhe(2-F)-Thr-Ser-Asp-Bip- | C(O)NH-R_{Y18} | 1.4 |
| | | Bip | | |
| 4-63 | H₂N | His-α-MePro-Glu-Gly-Thr-α-MePhe(2-F)-Thr-Ser-Asp-Bip- | C(O)NH-R_{Y19} | 1.2 |
| | | Bip | | |
| 4-64 | H₂N | His-α-MePro-Glu-Gly-Thr-α-MePhe(2-F)-Thr-Ser-Asp-Bip- | C(O)NH.R_{Y20} | 453 |
| | | Bip | | |
| 4-65 | H₂N | His-α-MePro-Glu-Gly-Thr-α-MePhe(2-F)-Thr-Ser-Asp-Bip- | C(O)NH-R_{Y21} | 3.8 |
| | | Bip | | |
| 4-66 | H₂N | His-α-MePro-Glu-Gly-Thr-α-MePhe(2-F)-Thr-Ser-Asp-Bip- | C(O)NH-R_{Y22} | 2.9 |
| | | Bip | | |
| 4-67 | H₂N | His-α-MePro-Glu-Gly-Thr-α-MePhe(2-F)-Thr-Ser-Asp-Bip- | C(O)NH-R_{Y23} | 2.4 |
| | | Bip | | |
| 4-68 | H₂N | His-α-MePro-Glu-Gly-Thr-α-MePhe(2-F)-Thr-Ser-Asp-Bip- | C(O)NH-R_{Y24} | 2.6 |
| | | Bip | | |
| 4-69 | H₂N | His-α-MePro-Glu-Gly-Thr-α-MePhe(2-F)-Thr-Ser-Asp-Bip- | C(O)NH-R_{Y25} | 5.1 |
| | | Bip | | |
| 4-70 | H₂N | His-α-MePro-Glu-Gly-Thr-α-MePhe(2-F)-Thr-Ser-Asp-Bip- | C(O)NH₂ | 0.004 |
| | | FN3(C18 acid) [NOTE: mixture of Click isomers 1 and 2) | | |
| 4-70-i1 | H₂N | His-α-MePro-Glu-Gly-Thr-α-MePhe(2-F)-Thr-Ser-Asp-Bip- | C(O)NH₂ | 0.04 |
| | | FN3(C18 acid) [Click isomer 1) | | (0% albumin) |
| 4-70-i2 | H₂N | His-α-MePro-Glu-Gly-Thr-α-MePhe(2-F)-Thr-Ser-Asp-Bip- | C(O)NH₂ | 0.7 |
| | | FN3(C18 acid) [Click isomer 2) | | (0% albumin) |
| 4-71 | H₂N | His-α-MePro-Glu-Gly-Thr-α-MePhe(2-F)-Thr-Ser-Asp- | C(O)NH₂ | 0.008 |
| | | FN3(C18 acid)-Bib [NOTE: mixture of Click isomers 1 | | |
| | | and 2) | | |
| 4-71-i1 | H₂N | His-α-MePro-Glu-Gly-Thr-α-MePhe(2-F)-Thr-Ser-Asp- | C(O)NH₂ | 0.009 |
| | | FN3(C18 acid)-Bip [Click isomer 1) | | (0% albumin) |

| | | | | |
|---|---|---|---|---|
| 4-71-i2 | H₂N | His-α-MePro-Glu-Gly-Thr-α-MePhe(2-F)-Thr-Ser-Asp- | C(O)NH₂ | 0.1 |
| | | FN3(C18 acid)-Bip [Click isomer 2) | | (0% albumin) |
| 4-72-i1 | H₂N | His-α-MePro-Glu-Gly-Thr-α-MePhe(2F)-Thr-Ser-Asp-α- | C(O)NH₂ | 0.2 |
| | | MeBip-Bip [Q-MeBip isomer 1] | | (0% albumin) |
| 4-72-i2 | H₂N | His-α-MePro-Glu-Gly-Thr-α-MePhe(2F)-Thr-Ser-Asp-α- | C(O)NH₂ | >1 |
| | | MeBip-Bip [D-α-MeBip isomer 2] | | (0% albumin) |
| 4-73-i1 | H₂N | His-α-MePro-Glu-Gly-Thr-α-MePhe(2F)-Thr-Ser-Asp-Bip-α- | C(O)NH₂ | 0.05 |
| | | MeBip [Q-MeBip isomer 1] | | (0% albumin) |
| 4-73-i2 | H₂N | His-α-MePro-Glu-Gly-Thr-α-MePhe(2F)-Thr-Ser-Asp-Bip-α- | C(O)NH₂ | 0.2 |
| | | MeBip [D-α-MeBip isomer 2] | | (0% albumin) |

### Example 3: Structure-Based Design and Computational Modeling Methods

The general approach to hypothesis-driven peptide design and many one of the computational methods described below have been described in recent publications (24-27) and have been successfully applied to diverse protein targets (e.g., IL2R, CXCR4, TLR2 and p53).

Comparative structural analysis and molecular modeling studies were carried out primarily using the cryo-EM structure of a 10-amino acid peptide in complex with full length GLP-1R [5nx2] (28). This structure was supplemented with complexes bound to Exendin P5 [6b3j] (29), GLP-1 [5vai] (30) and [6×18] (31), small-molecules [6×19 and 6×1a] (31), [6orv](32), [6xos] (33), [7c2e] (34), and [71ci, 71cj, 71ck] (35), and allosteric modulators [6vcb] (36).

Computational design of novel GLP-1 peptide analogs was accomplished using a hypothesis-driven design approach. This required that we build initial structural models of key 11-amino acid peptides positioned in the GLP-1R active site. This task was accomplished by building a model of the 11-amino acid lead peptide using the available X-ray and EM structures as templates. This was followed by constrained conformational optimization. Promising sites for peptide mutation were then identified and appropriate canonical or non-canonical amino acid libraries defined and built. Structures of novel peptides were then constructed by mutation of the peptide ligands from the above complexes using a computational program implemented in Python and employing the YASARA molecular modeling program (37). The resulting poses were refined either using molecular docking with VINA (38), a local conformational sampling routine in Python/YASARA (24) or using a proprietary Monte Carlo conformational search program (Sampler) written in C++ (24). When building analogs with considerable structural difference from the reference complexes, the initial poses created via the mutation program were subject to a short molecular dynamics relaxation step in which the protein backbone was held fixed. Designed analogs were subject to re-scoring, as appropriate. Rescoring was done by calculating MM and MM/PBSA binding energies. In addition, models were visually inspected to ensure they were not biased by artifacts of the calculation methods.

Models were qualitatively and quantitatively analyzed against experimentally measured cAMP EC50 results to enhance structural understanding of the GLP-1R binding to peptides and inform future design rounds. Structure-activity data was visually analyzed to identify patterns. Structure-activity data was also subject to quantitative structure-activity relationship (QSAR) analysis using ligand-based and receptor-ligand-based approaches. One ligand-based approach employed molecular field analysis using Cresset Forge (39). Another ligand-based approach employed molecular field analysis using Cresset Forge (https://www.cresset-group.com/sofware/forge/). For receptor-ligand QSAR analysis, receptor-ligand interface descriptors were calculated using proprietary YASARA scripts.

Initial QSAR analysis was performed on selected structure-activity relationship data. In particular, various receptor-based and ligand-based descriptors were calculated for 28 GLP-1 peptide/GLP-1R structural variants using an GLP-1 peptide/GLP-1R all-atom structural model based on the 5nx2 crystal structure. Multiple linear regression analysis was performed using pEC50 measurements as the dependent variables. The GLP-1 peptide variants covered Ala-scan, Aib-scan, truncated analogs, and N-terminal extension analogs (vide supra). The final descriptor-based regression equation includes three molecular descriptors and is given by,

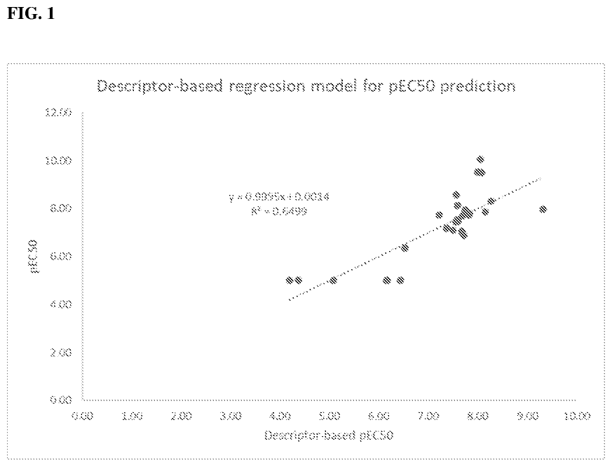

pEC50=−0.012YSCORE+−0.91Ion-IonEnergy+−0.19BackboneTorsions+Constant

where all descriptors were calculated using the YASARA molecular modeling software package, YSCORE refers the binding energy calculated using the NOVA2 forcefield, Ion-Ion refers to the electrostatic energy between ion-ion interface contacts, and BackboneTorsions refers to the total number of peptide phi/psi torsions (40). The overall model was found to be statistically significant. Individual terms were also found to make a statistically significant contribution to pEC50 estimation. The best fit line results are presented in FIG. 1. The results (R²=0.65) are encouraging, especially given that the QSAR equation is based on only three a priori physically and structurally reasonable descriptors that make statistically significant contributions to pEC50 estimation. Such work will focus on future training and testing of the QSAR model.

### Example 4: Peptide Synthesis

The polypeptides of the present invention were prepared using the below methods to couple the appropriate amino acids. Deprotection, cleavage and purification methods are also described.

### Peptide Synthesis, Purification, and Analysis:

The solid-phase peptide synthesis was achieved by standard methods. Typically, Amphispheres 40 RAM, 75-150 μM resin (Agilent Technologies) was used to generate peptides as C-terminal carboxamides. Amino acid coupling protocols using HCTU generally included the following four steps: (a) 1^{st} coupling—5 eq of amino acid (0.34 M), 10 eq DIEA (2 M), 5 eq of HCTU (0.5 M), 5 eq of 6-C1-HOBt (0.5 M), 30 minutes; (b) 2^{nd} coupling—5 eq of amino acid (0.34 M), 10 eq DIEA (2M), 5 eq of HCTU (0.5 M), 5 eq of 6-C1-HOBt (0.5 M), 90 minutes; (c) one DMF wash between couplings; and (d) nine DMF washes after second coupling. Amino acid coupling protocols using HATU generally included the following two steps: (a) single coupling −2 eq of amino acid (0.1 M), 4 eq DIEA (2M), 2 eq of HATU (0.5M), 5 eq of HOAt (0.5M), 240 minutes; and (b) nine DMF washes after coupling. Amino acid coupling protocols using PyOxim and HATU generally included the following four steps: (a) 1^{st} coupling—5 eq of amino acid (0.34 M), 10 eq DIEA (2 M), 5 eq of PyOxim (0.5 M), 120 minutes; (b) 2^{nd} coupling—5 eq of amino acid (0.34M), 10 eq DIEA (2M), 5 eq of HCTU (0.5M), 5 eq of HOAt (0.5M), 120 minutes; (c) one DMF wash between couplings; (d) nine DMF washes after second coupling. Fmoc deprotection protocols generally included the following three steps: (a) 20% piperdine in DMF, 10 minutes; (b) 20% piperdine in DMF, 15 minutes; and (c) Eight DMF washes. Cleavage of the amino acid side chain protecting groups and the peptide from the resins was typically accomplished by the following five steps: (a) 87.5% TFA, 2.5% anisole, 5% water, 5% triisopropylsilane, 3-4 hours, 10 mL of cleavage cocktail per 1 gram of resin; (b) a modified procedure for sulfur containing amino acids: 85% Tfa, 2.5% 3,6-dioxa-1,8-octanedithiol, 2.5% anisole, 5% water, 5% triisopropylsilane, 3-4 hours, 10 mL of cleavage cocktail per 1 gram of resin; (c) evaporate TFA; (d) precipitate with cold diethyl ether (minimum of 10:1, ether:cleavage cocktail) and centrifuge at 3000 rpm for 5 minutes, and then decant the ether (this was repeated three times); and e) peptide powder/pellets were then dried overnight. Purification by reversed-phase HPLC was achieved by the following four steps: (a) dissolve peptide; (b) chromatography using Biotage Selekt instrument and Biotage Sfar Bio C18 D (Duo, 300 Å, 20 μm): (c) pooling of desired fractions, freezing and lyophilization; and (d) 50% acetonitrile/water as then added to dry peptide and it was re-frozen and re-lyophilized. Analysis of the purified peptides was achieved by the following three steps: (a) a sample of peptide was dissolved and analyzed using an Agilent Infinity II LC/MS; (b) analysis for purity (214 and 280 nm absorbance detection) and retention time using an Agilent Zorbax 300SB—C18 (5 μm, 2.1×150 mm) and two solvent system consisting of A (0.1% TFA in water) and B (0.1% Tfa in acetonitrile), a gradient of 5-65% B over 20 minutes at 40° C.; and MS analysis using an Agilent Infinity Lab MSD, positive polarity (with Mass detection range is 100 to 1500).

### Amino Acids Exempiifying S-Perfluoroarylation (e.g., Cys-S-Biphenyl[F9]):

Dissolve the crude or purified Cys-containing peptide in DMF, to give a 1 mM concentration, that contains an excess of the electrophile (e.g., decafluorobiphenyl) and base such as N,N-diisopropylethylamine (DIEA) or TRIS. Upon completion of the reaction, quench with thiol, and purify by high-performance liquid chromatography (HPLC).

### Novel Amino Acids Exemplifying Azide/Alkyne Huisgen Cycloaddition (“Click”):

Procedure for peptide conjugation by azide/alkyne Huisgen cycloaddition: In a glass scintillation sealed with a septum cap add peptide azide and lipid-alkyne along with and copper bromide. Use nitrogen to purge the reaction mixture for 5 minutes to ensure the removal of oxygen and then add ˜1 mL of degassed DMF. Vortex the reaction mixture. Let the reaction continue for 2 hours and then purify by RP-HiPLC.

Resolute Bio's SOP—0002 specifies for the on-resin cycloaddition reaction using CuAAC reagent between the Phe(4-azide) at either position #10 or position #11 of the protected, fully assembled peptide-resin and an alkyne-fatty acid (e.g., 17-octadecynoic acid) at 0.1 mmol scale the following synthetic steps prior to purificaton by RP-HPLC: (1) Swell resin with DMF; (2) Wash resin 3 times with 20% 2,6-lutidine in DMF; (3) Add 1.5 equivalents of the alkyne reagent (or azido reagent if coupling to alkyne); (4) Add 49.5 mg of sodium ascorbate; (5) Add 45 μL of DIEA; (6) Add 47.5 mg CuI; and (7) Stir at ambient temperature overnight. Purification of RXL-4042-2 and RXL-4043-2 was achieved with the Agilent 1290 Preparative system with an Agilent 1260 Multiple Wavelength Detector. The fraction trigger was set to a wavelength of 214 nm. The reversed-phase chromatography column was an Agilent Prep, 100A, 5 um, C18, 50×21.2 mm. The structure of 17-octadecynoic acid is shown below:

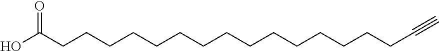 [SMILES: C#CCCCCCCCCCCCCCCCC(=O)O]

### Novel PK-Modified Peptides Exemplifying Albumin-Binding Functionality Conjugation:

Procedure for peptide conjugation with PK modifiers, e.g. serum albumin binding group: The N-terminus of the peptide requires acetylation or a Boc protecting group to prevents an amide formation at two primary amine locations (e.g., His¹ versus linker Dap [diaminoproprionic acid] or Glu amino group as represented within R_{Y2}-R_{Y12}. It is noted that such PK modifiers can be conjugated to either L- or D-enantiomers of Dap or Glu as well as other linker-related amino acids, including Lys, Om (ornithine) or Dab (diaminobutyric acid). In such cases, the primary amine moiety is protected with Mtt (methyltrityl) or Mmt (methoxytrityl) by the following protocol: (a) Wash the resin 3 times with 2% trifluoroacetic acid, 2% triisopropylsilane, and 96% DCM; (b) Shake with 2% trifluoroacetic acid, 2% triisopropylsilane, and 96% DCM for 30 minutes twice. Wash three times with DCM (dichloromethane). After Mtt or Mmt removal, wash the resin three times with 2% DIEA (diisopropylethylamine). Wash three times with DMF (N,N-dimethylforamide). Wash three times with NMP (N-methyl-2-pyrrolidinone). The amine can then be elaborated by direct conjugation with the PK modifiers (e.g., C18 fatty acid or aryl-halide) or linker groups (e.g., AEEA) generally using 2 equivalents (PK modifier or AEEA/amino acid linker) with 2 equivalents of PyOxim and 4 equivalents of DIEA. To test for completeness of coupling, a microcleave of peptide-resin may be performed to determine if further re-coupling steps are required.

### Example 5: In Vitro Pharmacology Screening Methods

Human embryonic kidney cells (HEK) co-expressing the hGLP1 receptor and CRE-Luciferase construct were used to determine agonist potency in this assay. The cells were thawed briefly at 37° C., transferred to a sterile tube and re-suspended in complete media at 37° C. Cells were centrifuged at 1000 rpm for 5 minutes and cells collected; cells were re-suspended in assay buffer consisting of DPBS (GIBCO) with 500 μM of the phosphodiesterase inhibitor IBMX. Assay medium could contain serum albumin (2%) to test for albumin affinity, or albumin-free as specified in particular protocols. The optimal cell density was determined to be 1000 cells/well; cells were added to wells in 384-well plates containing appropriate pre-prepared dilutions of compounds (test peptides or reference compound exendin-4), sealed and incubated with CO₂ for 30 min. Test peptide solutions were diluted from 10 mM stock solutions; for most peptides an initial run was performed in duplicate from a maximal concentration of 1.0 μM, with 11 concentrations tested for each peptide using serial 1:3 dilution from this maximal concentration. With peptides that were found to be particularly potent agonists, a subsequent assay was run using a maximal concentration of 1.0 nM (11 concentrations, 1:3 dilution from 1 nM). The agonist assay was a homogeneous time-resolved fluorescence (HTRF) assay (Cisbio).

Following incubation of the cells for 30 minutes with test or reference peptides, 5 μL of the cAMP acceptor cAMP-d2, prepared previously as a working solution from frozen stock (1:20 dilution), was added to each well of the assay plate, along with 5 μL of anti-cAMP antibody-cryptate working solution (diluted 1:20 from frozen stock). The wells were incubated for 1 hour at room temperature, and fluorescence was then read at 665 and 615 nm with an Envision reader with TRF laser. Data were saved and analyzed using Prism software (GraphPad). Concentration-response analysis was performed using 4-parameter logistic fits of the resulting data, and EC₅₀ values obtained for each test and reference compound.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES CITED

1. D. L. Williams. The diverse effects of brain GLP-1 receptors on ingestive behavior. Br. J. Pharmacol., 2021 (May 14, online ahead of print).
2. J. Ard et al. and L. Herman. Weight loss and maintainance related to the mechanism of action of glucagon-like peptide 1 receptor agonists. Adv. Ther., 2021 (May 11, online ahead of print).
3. A. Anderson, F. K. Knop and T. Vilsball. A pharmacological and clinical overview of oral semaglutide for the treatment of type 2 diabetes. Drugs, 2021 (May 8, online ahead of print).
4. B. Jones. The therapeutic potential of GLP-1 receptor biased agonism. Br. J. Pharmacol., 2021 (April 20, online ahead of print).
5. N. Tanday, P. R. Flatt and N. Irwin. Metabolic response and benefits of glucagon-like peptide-1 (GLP-1) receptor ligands. Br. J. Pharmacol., 2021 (April 7, online ahead of print).
6. J. M. Trujillo, W. Nuffer and B. A. Smith. GLP-1 receptor agonists: An updated review of head-to-head clinical studies. Ther. Adv. Endocrinol. Metab., 12, 1-15, 2021.
7. W. Widiarti et al. and F. F. Alkaff. Cardioprotective properties of glucagon-like peptide-1 receptor agonist in type 2 diabetes mellitus patients: A systematic review. Diabetes Metab. Syndr., 15, 837-843, 2021.
8. P. Richard, N. A. Thornberry and S. Pinto. The gut-brain axis: Identifying new therapeutic approaches for type 2 diabetes, obesity, and related disorders. Mol. Metab., 46, 1-19, 2021.
9. F. M. Gribble and F. Reimann. Metabolic messengers: Glucagon-like peptide 1. Nature Metab., 3, 142-148, 2021.
10. J. Helmstadter et al. and S. Steven. Glucagon-like peptide-1 (GLP-1) receptor agonists and their cardiovascular benefits: The role of the GLP-1 receptor. Br. J. Pharmacol., 2021 (March 25, online ahead of print).
11. J. E. Hunt et al. and H. Kissow. GLP-1 and intestinal diseases. Biomedicines, 9, 383, 2021.
12. A. Shandilya and S. Mehan. Dysregulation of IGF-1/GLP-1 signaling in the progression of ALS: Potential target activators and influences on neurological disfunctions. Neurol. Sci., 2021 (May 21, online ahead of print).
13. J. Detka and K. Glombik. Insights into possible role of glucagon-like peptide-1 receptor agonists in the treatment of depression. Pharmacol. Rep., 2021 (May 18, online ahead of print).
14. C. Holscher. Protective properties of GLP-1 and associated peptide hormones in neurodegenerative disorders. Br. J. Pharmacol., 2021 (April 26, online ahead of print).
15. C. M. Labandeira et al, and A. Rodriguez-Perez. Diabetes, insulin and new therapeutic strategies for Parkinson's disease: Focus on glucagon-like peptide-1 receptor agonists. Front. Neuroendocrinol., 62, 100914, 2021.
16. S. T. Ferreira. Brain insulin, insulin-like growth factor 1 and glucagon-like peptide 1 signalling in Alzheimer's disease. JNeuroendocrinol., 33, e12959, 2021.
17. L. B. Knudsen and J. Lau. The discovery and development of liraglutide and semaglutide. Frontiers in Endocrinology, 10 (article 155), 1-32, 2019.
18. L. B. Knudsen et al. and H. Agerso. Potent derivatives of glucagon-like-peptide 1 with pharmacokinetic properties suitable for once daily administration. J. Med. Chem., 43, 1664-1669, 2000.
19. J. Lau et al. and T. Kruse. Discovery of the once-weekly glucagon-like peptide-1 (GLP-1) analogue semaglutide. J. Med. Chem., 58, 7370-7380, 2015.
20. C. E. Dumelin et al. and D. Neri. A portable albumin binder from a DNA-encoded chemical library. Angew. Chm. Int. Ed., 47, 3196-3201, 2008.
21. C. Mapelli et al. and W. R. Ewsing. Eleven amino acid glucagon-like peptide-1 receptor agonists with antidiabetic activity. J. Med. Chem., 52, 7788-7799, 2009.
22. T. S. Haque et al. and J. Krupinski. Identification of potent 11-mer glucagon-like peptide-1 receptor peptides with novel C-terminal amino acids: Homohomophenylalanine analogs. Peptides, 31, 950-955, 2010.
23. T. S. Haque et al. and J. Krupinski. Exploration of structure-activity relationships at the two C-terminal residues of potent 11-mer glucagon-like peptide-1 receptor agonist peptides via parallel synthesis. Peptides, 31, 1353-1360, 2010.
24. D. J. Diller et al. and J. Audie. Rigorous computational and experimental investigations on MDM2/MDMX-targeted linear and macrocyclic peptides. Molecules, 24, 4586, 2019.
25. J. Swanson and J. Audie. An unexpected way forward: Towards a more accurate and rigorous protein-protein binding affinity scoring function by eliminating terms from an already simple scoring function. J. Biomol. Struct. Dyn., 36, 83-97, 2018.
26. D. J. Diller et al. and J Audie. Rational, computer-enabled peptide drug design: Principles, methods, applications and future directions. Future Med. Chem., 7, 2173-2193.
27. T. Price-Troska et al. and S. M. Ansell. Inhibiting IL-2 signaling and the regulatory T-cell pathway using computationally designed peptides. Invest. New Drugs, 37, 9-16, 2019.
28. A. Jazayeri et al. and F. H. Marshall. Crystal structure of the GLP-1 receptor bound to a peptide agonist. Nature, 546, 254-258.
29. Y-L. Liang et al. and D. Wootten. Phase-plate cryo-EM structure of a biased agonist-bound human GLP-1 receptor-Gs complex. Nature, 555, 121-125.
30. Y. Zhang et al. and G. Skiniotis. Cryo-EM structure of the activated GLP-1 receptor in complex with a G protein. Nature,546, 248-153.
31. X. Zhang et al. and D. Wooten. Differential GLP-1R binding and activation and peptide and non-peptide agonists. Mol. Cell., 80, 486-500, 2020.
32. P. Zhao et al. and D. Wooten. Activation of the GLP-1 receptor by a non-peptide agonist. Nature, 577, 432-436, 2020.
33. T. Kawai et al. and K. W. Sloop. Cryo-E of human GLP-1 bound to a non-peptide agonist LY3502970. Proc. Natl. Acad. Sci. U.S.A., 117, 29959-29967, 2020.
34. H. Ma et al. and H. E. Xu. GLP-1R-Gs complex structure with small molecule full agonist. Cell Res., 30, 1140-1142, 2020.
35. X. Zhang et al. and M. J. Belousoff. Evolving cryo-EM structural approaches for GPCR drug discovery. Structure, 29, 1-12, 2021.
36. A. B. Bueno et al. and K. W. Sloop. Structural insights into probe-dependent positive allosterism of the GLP-1 receptor. Nature Chem. Biol., 16, 1105-1110, 2020.
37. E. Krieger and G. Vriend. New ways to boost molecular dynamics simulation. J. Comput. Chem., 36, 996, 2015.
38. O. Trott and A. J. Olson. AutoDock Vina: Improving the speed and accuracy of docking with a new scoring function, efficient optimization, and mutlthreading. J. Comput. Chem., 31, 455-461, 2010.
39. T. J. Cheeseright, M. D. Mackey and R. A. Scoffin. High content pharmacophores from molecular fields: A biologically relevant method for comparing and understanding ligands. Curr. Comput. Aided Drug Des., 7, 190-205, 2011.
40. E. Krieger and G. Vriend. YASARA View: Molecular graphics for all devises from smartphones to workstations. Bioinformatics, 30, 2981-2982, 2014.

1. A polypeptide represented by the following sequence:
R_{XN}—Xₐₐ¹—Xₐₐ²—Xₐₐ³—Xₐₐ⁴—Xₐₐ⁵—Xₐₐ⁶—Xₐₐ⁷—Xₐₐ⁸—Xₐₐ⁹—Xₐₐ¹⁰—Xₐₐ¹¹—R_{YC}
wherein
R_{XN} is the N-terminal group of Xₐₐ^{l} selected from H (i.e., des-amino) and —N(Rx)₂, wherein Rx, independently for each occurrence, is H or an optionally substituted alkyl, arylalkyl, heteroarylalkyl, formyl, acetyl, alkanoyl, —C(O)-alkyloxy, —C(O)-aryloxy, —C(O)-aralkyloxy, —C(O)— heterocyclyloxy, —C(O)-heteroarylalkyloxy, —C(O)NH-alkyl, —C(O)NH-aryl, —C(O)NH-arylalkyl, —SO₂-heterocyclyl, —SO₂-alkyl, —SO₂-aryl, —SO₂-arylalkyl, —SO₂-heteroarylalkyl, —SO₂-heteroaryl, or ureido; or one occurrence of Rx is hydrogen and the other occurrence is an amino acid residue Xₐₐ⁰;
Xₐₐ⁰ is an optionally substituted amino acid residue selected from Gly, Pro, Arg, Glu, His, Phe, Trp, and Aib;
Xₐₐ^{l} is an optionally substituted amino acid residue comprising an amino acid side chain that comprises an alkyl, aryl or heteroaryl;
Xₐₐ² is an optionally substituted amino acid residue selected from Gly, Aib, Ala, D-Ala, N-methyl-Ala, N-methyl-D-Ala, Pro, α-methyl-Pro, Val, D-Val, and D-His;
Xₐₐ³ is an optionally substituted amino acid residue comprising an amino acid side chain that comprises a carboxyl or sulfonic acid group;
Xₐₐ⁴ is an amino acid residue selected from Gly, Ala, Aib, and β-Ala;
Xₐₐ⁵ is an optionally substituted amino acid selected from Thr, Ser, Ala, Aib, Val, α-MeSer, α-MeThr, and α-MeVal;
Xₐₐ⁶ is an optionally substituted amino acid residue that is disubstituted at the α carbon, provided that one of the substituents is an optionally substituted aryl or heteroaryl;
Xₐₐ⁷ is an optionally substituted amino acid residue comprising an amino acid side chain that comprises a hydroxyl;
Xₐₐ⁸ is an optionally substituted amino acid residue selected from Ser, His, and Asn;
Xₐₐ⁹ is an optionally substituted amino acid residue comprising an amino acid side chain that comprises a carboxyl or sulfonic acid group;
Xₐₐ¹⁰ is an optionally substituted amino acid residue comprising an amino acid side chain that comprises a sulfide and/or an optionally substituted aryl or heteroaryl;
Xₐₐ¹¹ is an optionally substituted amino acid residue comprising an amino acid side chain that comprises a sulfide and/or an optionally substituted aryl or heteroaryl; and
R_{YC} is the C-terminal group of Xₐₐ¹¹ having the structure —C(O)N(R_{Y})₂, wherein R_{Y}, independently for each occurrence, is hydrogen or a PK modifier group.

2. (canceled)

3. The polypeptide of claim 2, wherein
Xₐₐ^{l} is an optionally substituted amino acid residue selected from, Leu, His, and Tyr, wherein the amino acid residue, when substituted, is substituted with at least one halo, hydroxyl, or alkyl:
Xₐₐ² is an optionally substituted amino acid residue selected from Gly, Aib, Ala, D-Ala, N-methyl-Ala, N-methyl-D-Ala, Pro, (α-methyl-Pro, Val, and D-Val, wherein the amino acid residue is substituted with at least one halo or alkyl:
Xₐₐ³ is an amino acid residue selected from Asp, Glu, and cysteic acid, wherein the amino acid residue, when substituted, is substituted with at least one halo or alkyl:
Xₐₐ⁴ is an amino acid residue selected from Gly and Ala:
Xₐₐ⁵ is an optionally unsubstituted amino acid residue selected from Thr, Ser, Ala, Aib, Val, α-MeSer, α-MeThr, and α-MeVal, wherein Xₐₐ⁵ is substituted with at least one halo or alkyl:
Xₐₐ⁶ is selected from α-MePhe, α-MePhe(₂-F), and α-MePhe(2,6-DiF),
Xₐₐ⁷ is an optionally substituted amino acid residue selected from Thr, α-MeThr, Ser, and α-MeSer, wherein the amino acid residue, when substituted, is substituted with at least one halo or alkyl:
Xₐₐ⁸ is an optionally substituted amino acid residue selected from Ser, His, and Asn, wherein the amino acid residue, when substituted, is substituted with at least one halo or alkyl: and
Xₐₐ⁹ is an optionally substituted amino acid residue selected from Asp, Glu, and cysteic acid, wherein the amino acid residue, when substituted, is substituted with at least one halo or alkyl.

4.-33. (canceled)

34. The polypeptide of claim 1, wherein Xₐₐ¹⁰ is represented by: 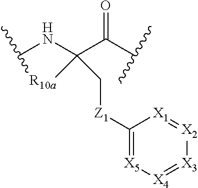
wherein
X₁ is N or CR_{10b};
X₂ is N or CR_{10c};
X₃ is N or CR_{10d};
X₄ is N or CR₁₀ₑ;
X₅ is N or CR_{10f};
Z₁ is absent or present, and when present is S or SO₂;
R₁₀ₐ is H or alkyl; and
R_{10b}, R_{10c}, R_{10d}, R₁₀ₑ, and R_{10f} are independently selected from H, halogen, and alkyl, or 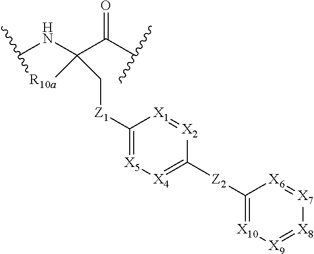
wherein
X₁ is N or CR_{10b};
X₂ is N or CR_{10c};
X₄ is N or CR₁₀ₑ;
X₅ is N or CR_{10f};
X₆ is N or CR_{10g};
X₇ is N or CR₁₀ₕ;
X₈ is N or CR₁₀ᵢ;
X₉ is N or CR₁₀ⱼ;
X₁₀ is N or R₁₀ₖ;
Z₁ is absent or present, and when present is S or SO₂:
Z₂ is absent or present, and when present is S or SO₂:
R₁₀ₐ is selected from H and alkyl; and
R_{10b}, R_{10c}, R₁₀ₑ, R_{10f}, R_{10g}, R₁₀ₕ, R₁₀ᵢ, R₁₀ᵢ, and R₁₀ₖ are independently selected from H, halogen, and alkyl.

35. (canceled)

36. The polypeptide of claim 34, wherein Xₐₐ¹⁰ is represented by:

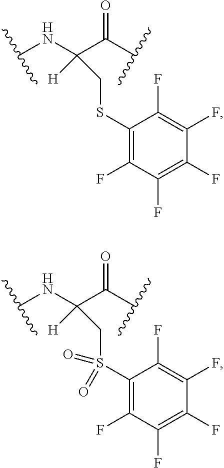 [SMILES: *NC(CSc1c(F)c(F)c(F)c(F)c1F)C(*)=O ; *NC(CS(=O)(=O)c1c(F)c(F)c(F)c(F)c1F)C(*)=O]

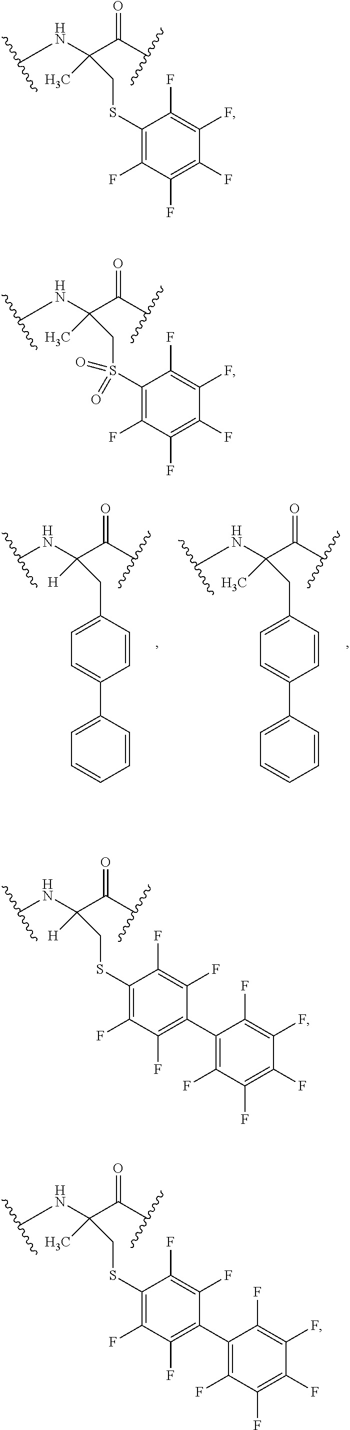 [SMILES: *NC(C)(CSc1c(F)c(F)c(F)c(F)c1F)C(*)=O ; *NC(C)(CS(=O)(=O)c1c(F)c(F)c(F)c(F)c1F)C(*)=O ; *NC(Cc1ccc(-c2ccccc2)cc1)C(*)=O ; *NC(C)(Cc1ccc(-c2ccccc2)cc1)C(*)=O ; *NC(CSc1c(F)c(F)c(-c2c(F)c(F)c(F)c(F)c2F)c(F)c1F)C(*)=O ; *NC(C)(CSc1c(F)c(F)c(-c2c(F)c(F)c(F)c(F)c2F)c(F)c1F)C(*)=O]

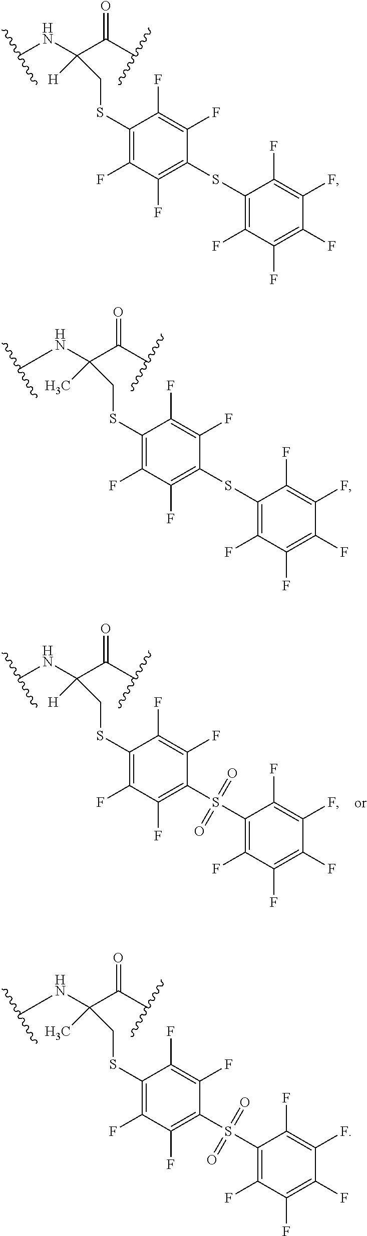 [SMILES: *NC(CSc1c(F)c(F)c(Sc2c(F)c(F)c(F)c(F)c2F)c(F)c1F)C(*)=O ; *NC(C)(CSc1c(F)c(F)c(Sc2c(F)c(F)c(F)c(F)c2F)c(F)c1F)C(*)=O ; *NC(CSc1c(F)c(F)c(S(=O)(=O)c2c(F)c(F)c(F)c(F)c2F)c(F)c1F)C(*)=O ; *NC(C)(CSc1c(F)c(F)c(S(=O)(=O)c2c(F)c(F)c(F)c(F)c2F)c(F)c1F)C(*)=O]

37.-39. (canceled)

40. The polypeptide of claim 1, wherein Xₐₐ¹⁰ is represented by: 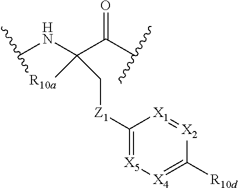
wherein
X₁ is N or CR_{10b};
X₂ is N or CR_{10c};
X₄ is N or CR₁₀ₑ;
X₅ is N or CR_{10f};
Z₁ is absent or present, and when present is selected from CH₂, S, O, NH, SO₂, SO₂—NH, NH—SO₂, NHC(O), and C(O)NH;
R₁₀ₐ is selected from H and alkyl;
R_{10d} is —L₁-L₂-L₃-R_{10d}′;
R₁₀ₐ′ is —NH₂, (C₁-C₂₀ alkyl)-CO₂H or optionally substituted (C₁-C₆ alkyl)-aryl;
L₁ is absent or present and when present is a linker;
L₂ is a linker which comprises an ether moiety;
L₃ is absent or present and when present is a linker that comprises an amino acid moiety; and
R_{10b}, R_{10c}, R_{10d}, R₁₀ₑ, and R_{10f} are independently selected from H, halogen, and alkyl, or 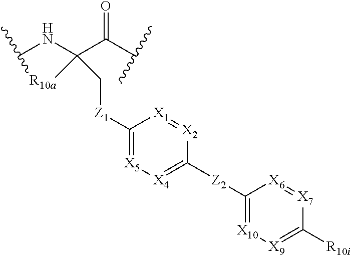
wherein
X₁ is N or CR_{10b};
X₂ is N or CR_{10c};
X₄ is N or CR₁₀ₑ;
X₈ is N or CR_{10f};
X₆ is N or CR_{10g};
X₇ is N or CR₁₀ₕ;
X₉ is N or CR₁₀ⱼ;
X₁₀ is N or CR₁₀ₖ:
Z₁ is absent or present, and when present is selected from CH₂, NH, S, So₂, O, SO₂—NH, NH—SO₂, NHC(O), and C(O)NH:
Z₂ is absent or present, and when present is selected from NH, S, So₂, O, SO₂—NH, NH—SO₂, NHC(O), and C(O)NH:
R₁₀ₐ is selected from H and alkyl:
R₁₀ᵢ is —L₁-L₂-L₃-R₁₀ᵢ′;
R₁₀ᵢ′ is —NH₂, (C₁-C₂₀ alkyl)-CO₂H or optionally substituted (C₁-C₆ alkyl)-aryl:
L₁ is absent or present and when present is a linker:
L₂ is a linker which comprises an ether moiety;
L₃ is absent or present and when present is a linker that comprises an amino acid moiety; and
R_{10b}, R_{10c}, R₁₀ₑ, R_{10f}, R_{10g}, R₁₀ₕ, R₁₀ᵢ, and R₁₀ₖ are independently selected from H, halogen, and alkyl.

41. (canceled)

42. The polypeptide of claim 1, Xₐₐ¹⁰ is represented by: 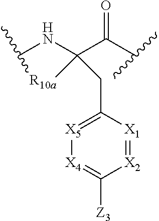
wherein
X₁ is N or CR_{10b};
X₂ is N or CR_{10c};
X₄ is N or CR₁₀ₑ;
X₈ is N or CR_{10f};
R₁₀ₐ is selected from H and alkyl;
R_{10b}, R_{10c}, R₁₀ₑ, and R_{10f} are independently selected from H, halogen, and alkyl; and
Z₃ is a substituted heteroaryl.

43.-45. (canceled)

46. The polypeptide of claim 1, wherein Xₐₐ¹⁰ is represented by:

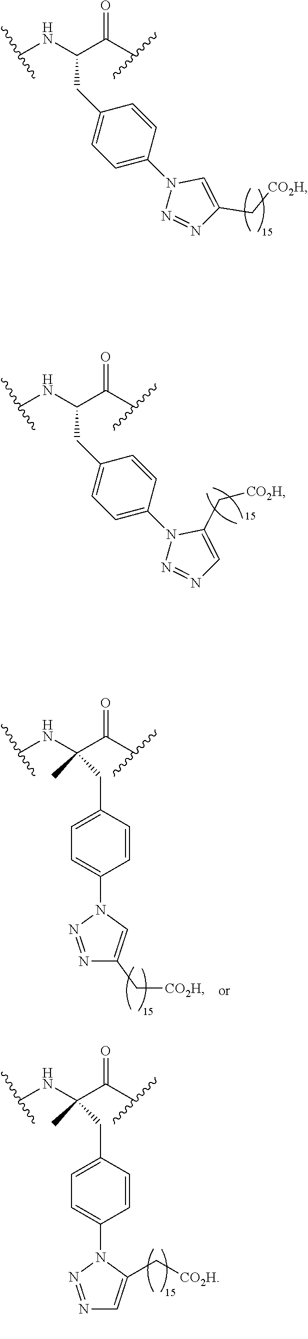

47. The polypeptide of claim 1, wherein Xₐₐ¹¹ is represented by: 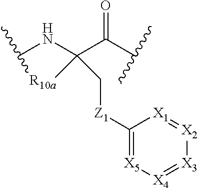
wherein
X₁ is N or CR_{10b};
X₂ is N or CR_{10c};
X₃ is N or CR_{10d};
X₄ is N or CR₁₀ₑ;
X₅ is N or CR_{10f};
Z₁ is absent or present, and when present is S or SO₂;
R₁₀ₐ is selected from H and alkyl; and
R_{10b}, R_{10c}, R_{10d}, R₁₀ₑ, and R_{10f} are independently selected from H, halogen, and alkyl, or 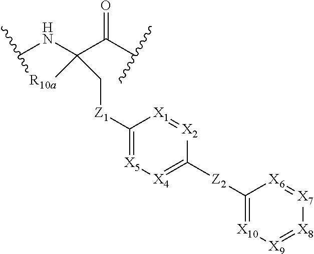
wherein
X₁ is N or CR_{10b};
X₂ is N or CR_{10c};
X₄ is N or CR₁₀ₑ;
X₈ is N or CR_{10f};
X₆ is N or CR_{10g};
X₇ is N or CR₁₀ₕ;
X₈ is N or CR₁₀ᵢ;
X₉ is N or CR₁₀ⱼ;
X₁₀ is N or CR₁₀ₖ;
Z₁ is absent or present, and when present is S or SO₂:
Z₂ is absent or present, and when present is S or SO₂:
R₁₀ₐ is selected from H and alkyl; and
R_{10b}, R_{10c}, R₁₀ₑ, R_{10f}, R_{10g}, R₁₀ₕ, R₁₀ᵢ, R₁₀ᵢ, and R₁₀ₖ are independently selected from H, halogen, and alkyl.

48. (canceled)

49. The polypeptide of claim 48, wherein Xₐₐ¹¹ is represented by:

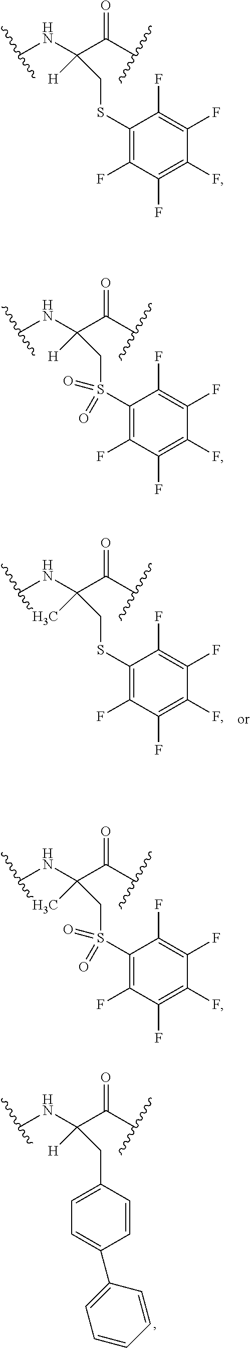 [SMILES: *NC(CSc1c(F)c(F)c(F)c(F)c1F)C(*)=O ; *NC(CS(=O)(=O)c1c(F)c(F)c(F)c(F)c1F)C(*)=O ; *NC(C)(CSc1c(F)c(F)c(F)c(F)c1F)C(*)=O ; *NC(C)(CS(=O)(=O)c1c(F)c(F)c(F)c(F)c1F)C(*)=O ; *NC(Cc1ccc(-c2ccccc2)cc1)C(*)=O]

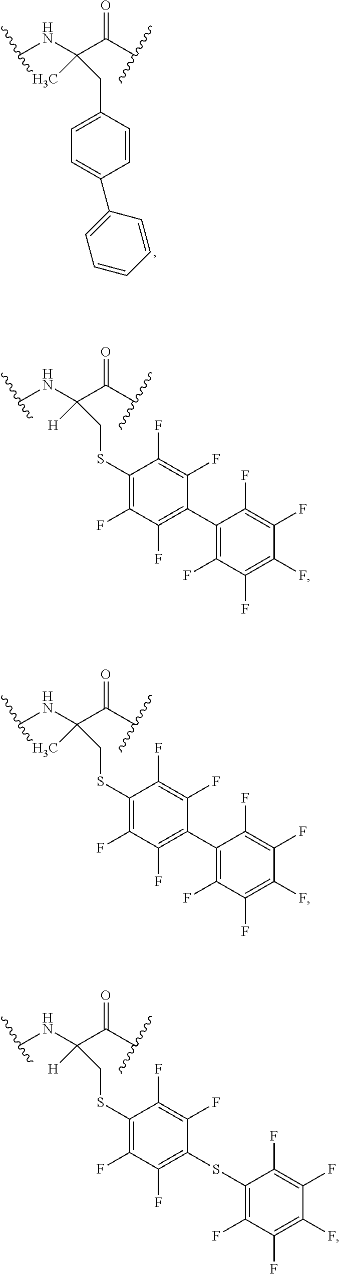 [SMILES: *NC(C)(Cc1ccc(-c2ccccc2)cc1)C(*)=O ; *NC(CSc1c(F)c(F)c(-c2c(F)c(F)c(F)c(F)c2F)c(F)c1F)C(*)=O ; *NC(C)(CSc1c(F)c(F)c(-c2c(F)c(F)c(F)c(F)c2F)c(F)c1F)C(*)=O ; *NC(CSc1c(F)c(F)c(Sc2c(F)c(F)c(F)c(F)c2F)c(F)c1F)C(*)=O]

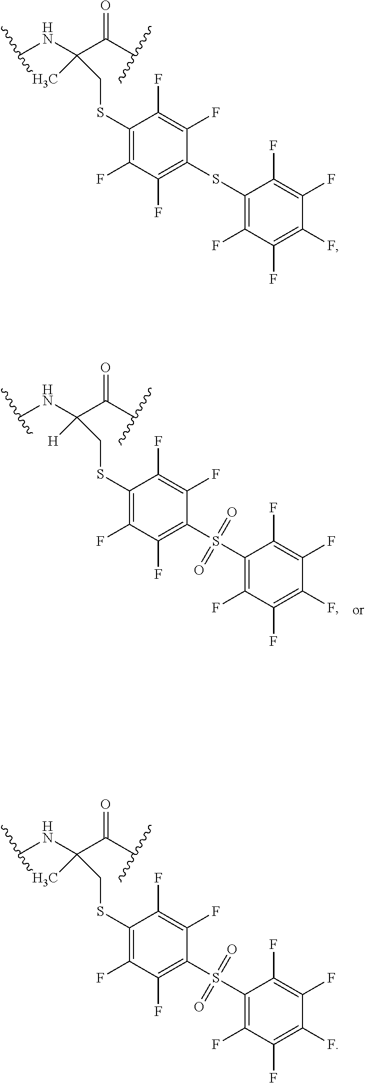 [SMILES: *NC(C)(CSc1c(F)c(F)c(Sc2c(F)c(F)c(F)c(F)c2F)c(F)c1F)C(*)=O ; *NC(CSc1c(F)c(F)c(S(=O)(=O)c2c(F)c(F)c(F)c(F)c2F)c(F)c1F)C(*)=O ; *NC(C)(CSc1c(F)c(F)c(S(=O)(=O)c2c(F)c(F)c(F)c(F)c2F)c(F)c1F)C(*)=O]

50.-52. (canceled)

53. The polypeptide of claim 1, wherein Xₐₐ¹¹ is represented by: 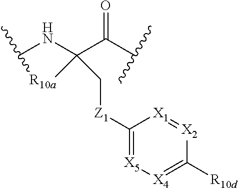
wherein
X₁ is N or CR_{10b};
X₂ is N or CR_{10c};
X₄ is N or CR₁₀ₑ;
X₅ is N or CR_{10f};
Z₁ is absent or present, and when present is selected from CH₂, S, O, NH, SO₂, SO₂—NH, NH—SO₂, NHC(O), and C(O)NH;
R₁₀ₐ is selected from H and alkyl;
R_{10d} is —L₁-L₂-L₃-R_{10d}′;
R_{10d}′ is —NH₂, (C₁-C₂₀ alkyl)-CO₂H or optionally substituted (C₁-C₆ alkyl)-aryl;
L₁ is absent or present and when present is a linker;
L₂ is a linker which comprises an ether moiety;
L₃ is absent or present and when present is a linker that comprises an amino acid moiety; and
R_{10b}, R_{10c}, R_{10d}, R₁₀ₑ, and R_{10f} are independently selected from H, halogen, and alkyl, or 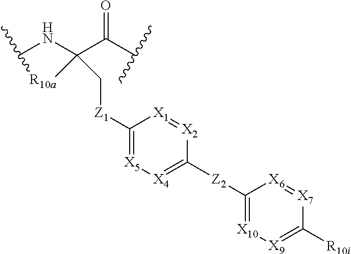
wherein
X₁ is N or CR_{10b};
X₂ is N or CR_{10c};
X₄ is N or CR₁₀ₑ;
X₅ is N or CR_{10f};
X₆ is N or CR_{10d};
X₇ is N or CR₁₀ₕ;
X₉ is N or CR₁₀ⱼ;
X₁₀ is N or CR₁₀ₖ;
Z₁ is absent or present, and when present is selected from CH₂, NH, S, SO₂, O, SO₂—NH, NH—SO₂, NHC(O), and C(O)NH:
Z₂ is absent or present, and when present is selected from NH, S, SO₂, SO₂—NH, NH—SO₂, NHC(O), and C(O)NH:
R₁₀ₐ is selected from H and alkyl:
R₁₀ᵢ is —L₁-L₂-L₃-R₁₀ᵢ′;
R₁₀ᵢ′ is —NH₂, (C₁-C₂₀ alkyl)-CO₂H or optionally substituted (C₁-C₆ alkyl)-aryl:
L₁ is absent or present and when present is a linker:
L₂ is a linker which comprises an ether moiety:
L₃ is absent or present and when present is a linker that comprises an amino acid moiety; and
R_{10b}, R_{10c}, R₁₀ₑ, R_{10f}, R_{10g}, R₁₀ₕ, R₁₀ᵢ, and R₁₀ₖ are independently selected from H, halogen, and alkyl.

54. (canceled)

55. The polypeptide of claim 1, Xₐₐ¹¹ is represented by: 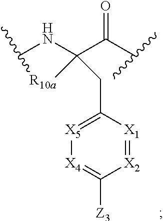
wherein
X₁ is N or CR_{10b};
X₂ is N or CR_{10c};
X₄ is N or CR₁₀ₑ;
X₅ is N or CR_{10f};
R₁₀ₐ is selected from H and alkyl;
R_{10b}, R_{10c}, R₁₀ₑ, and R_{10f} are independently selected from H, halogen, and alkyl; and
Z₃ is a substituted heteroaryl.

56.-58. (canceled)

59. The polypeptide of claim 1, wherein Xₐₐ¹¹ is represented by:

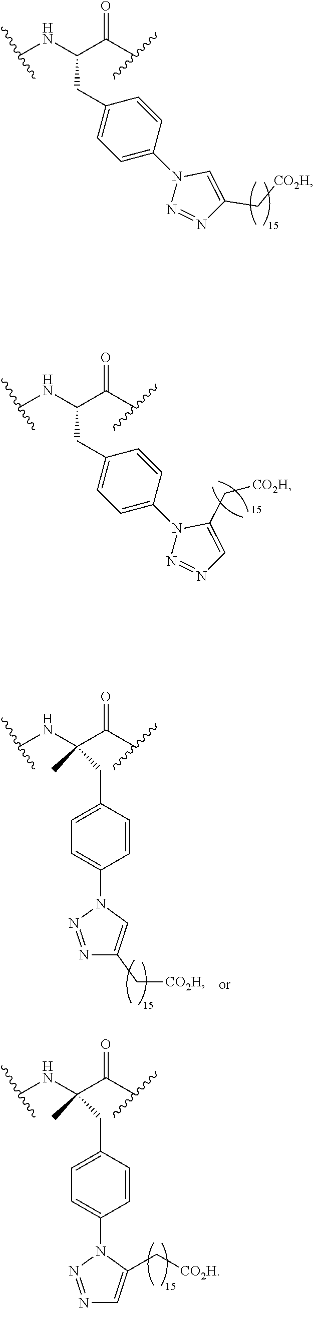

60.-70. (canceled)

71. The polypeptide of claim 1, wherein Xₐₐ¹⁰ is an optionally substituted amino acid residue selected from Phe, Tyr, Trp, homophenylalanine (Hph), homotyrosine (Hty), Bip, α-MeBip, 4-phenyl-3-pyridylalanine, 4-phenyl-4-pyridylalanine, α-MeHph, α-MeTyr, α-MeHty, Tyr(O-phenyl), Phe(4-S-phenyl), Phe(4—SO₂—NH-phenyl), Phe(4-CO—NH-phenyl), Cys(S-phenyl), Cys(S-phenyl[2,3,4,5,6-F₅]), Cys(S-pheny[2,3,4,5-F₄]-4-phenyl[2′,3′,4′,5′,6′-F₅]), Cys(S-pheny[2,3,4,5-F₄]-4—SO₂-phenyl[2′,3′,4′,5′,6′-F₅]), Cy(SO₂—NH-phenyl), Dap(3-C[═O]-phenyl), Dap(3-[C═O]-pyridyl), Asp(3-NH-phenyl), and Asp(3-NH-pyridyl).

72. The polypeptide of claim 1, wherein Xₐₐ¹¹ is an optionally substituted amino acid residue selected from Phe, Tyr, Trp, homophenylalanine (Hph), homotyrosine (Hty), Bip, α-MeBip, 4-phenyl-3-pyridylalanine, 4-phenyl-4-pyridylalanine, α-MeHph, α-MeTyr, α-MeHty, Tyr(O-phenyl), Phe(4-S-phenyl), Phe(4-SO₂—NH-phenyl), Phe(4-CO—NH-phenyl), Cys(S-phenyl), Cys(S-phenyl[2,3,4,5,6-F₅]), Cys(S-pheny[2,3,4,5-F₄]-4-phenyl[2′,3′,4′,5′,6′-F₅]), Cys(S-pheny[2,3,4,5-F₄]-4—SO₂-phenyl[2′,3′,4′,5′,6′-F₅]), Cy(SO₂—NH-phenyl), Dap(3-C[═O]-phenyl), Dap(3-[C═O]-pyridyl), Asp(3-NH-phenyl), and Asp(3-NH-pyridyl).

73. The polypeptide of claim 1, wherein R_{XN} is —N(Rx)₂, wherein each Rx is H.

74. (canceled)

75. The polypeptide of claim 1, wherein R_{YC} is —C(O)N(R_{Y})₂, wherein each R_{Y} is H.

76. The polypeptide of claim 1, wherein R_{YC} is —C(O)N(R_{Y})₂;
one occurrence of R_{Y} is hydrogen and the other occurrence of R_{Y} is —L₄-L₅-L₆-L₇-R_{Y}′;
R_{Y}′ is (C₁-C₂₀ alkyl)-CO₂H or optionally substituted (C₁-C₆ alkyl)-aryl;
L₄ is absent or present and when present is a linker that comprises an amino acid moiety;
L₅ is absent or present and when present is a linker that comprises an amino acid moiety;
L₆ is absent or present and when present is a linker that comprises an ether moiety; and
L₇ is a linker that comprises an amino acid moiety.

77.-84. (canceled)

85. The polypeptide of claim 1, wherein R_{YC} is —C(O)NHR_{Y}, wherein NHR_{Y} is selected from:

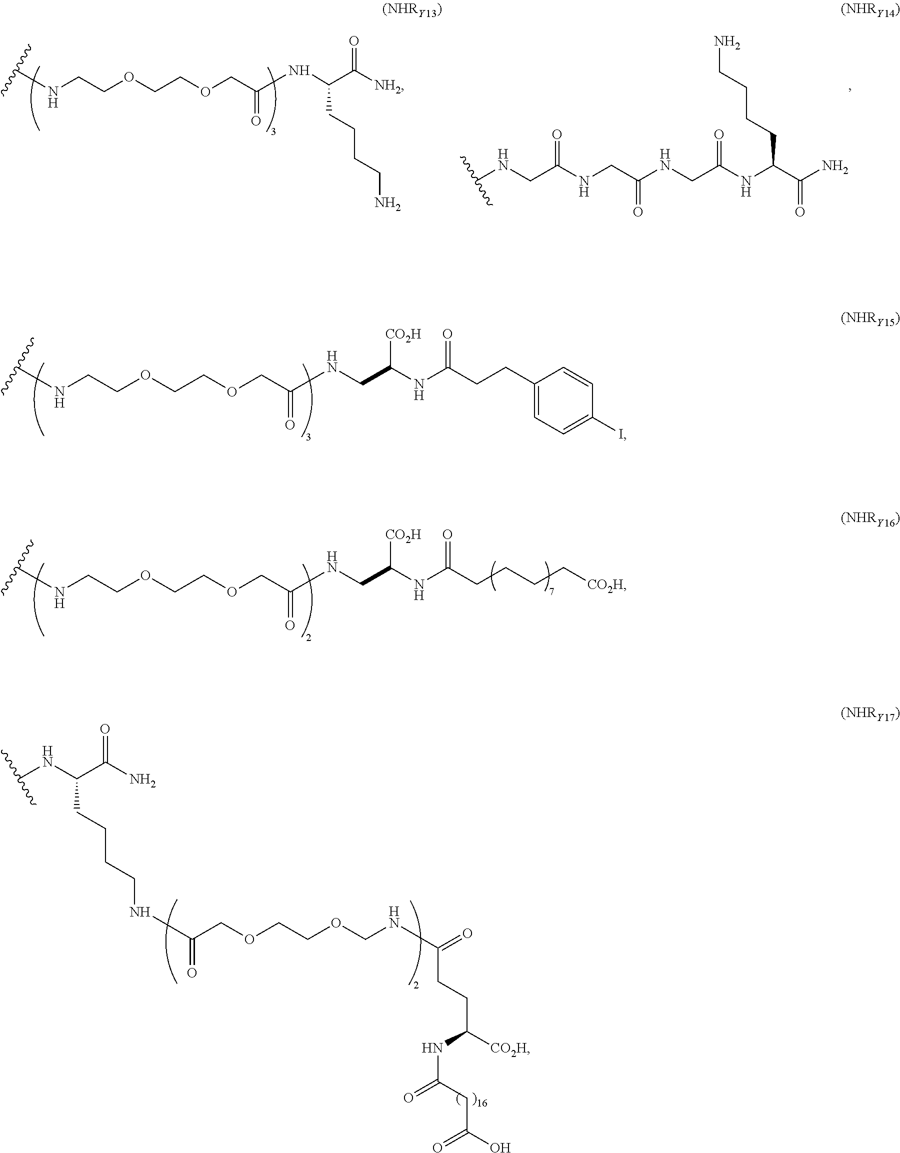

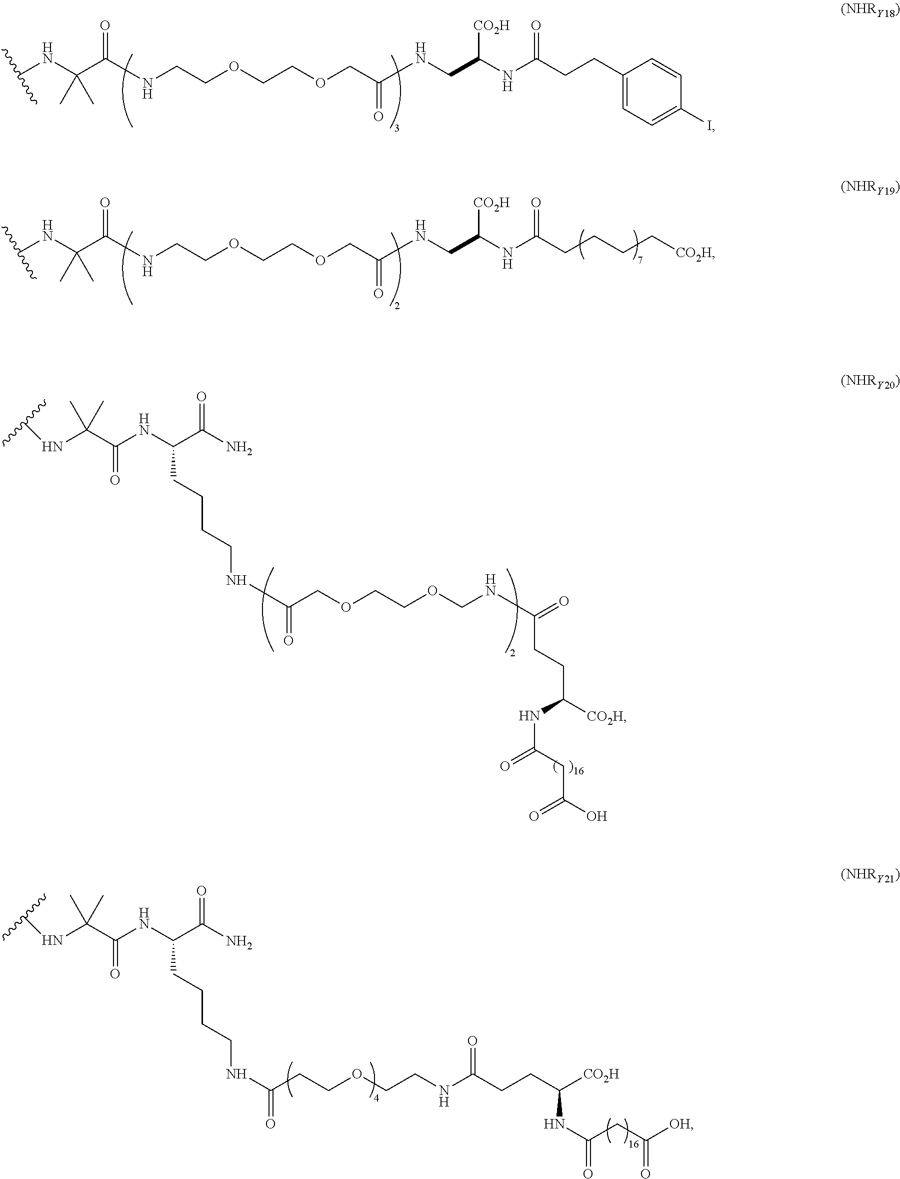

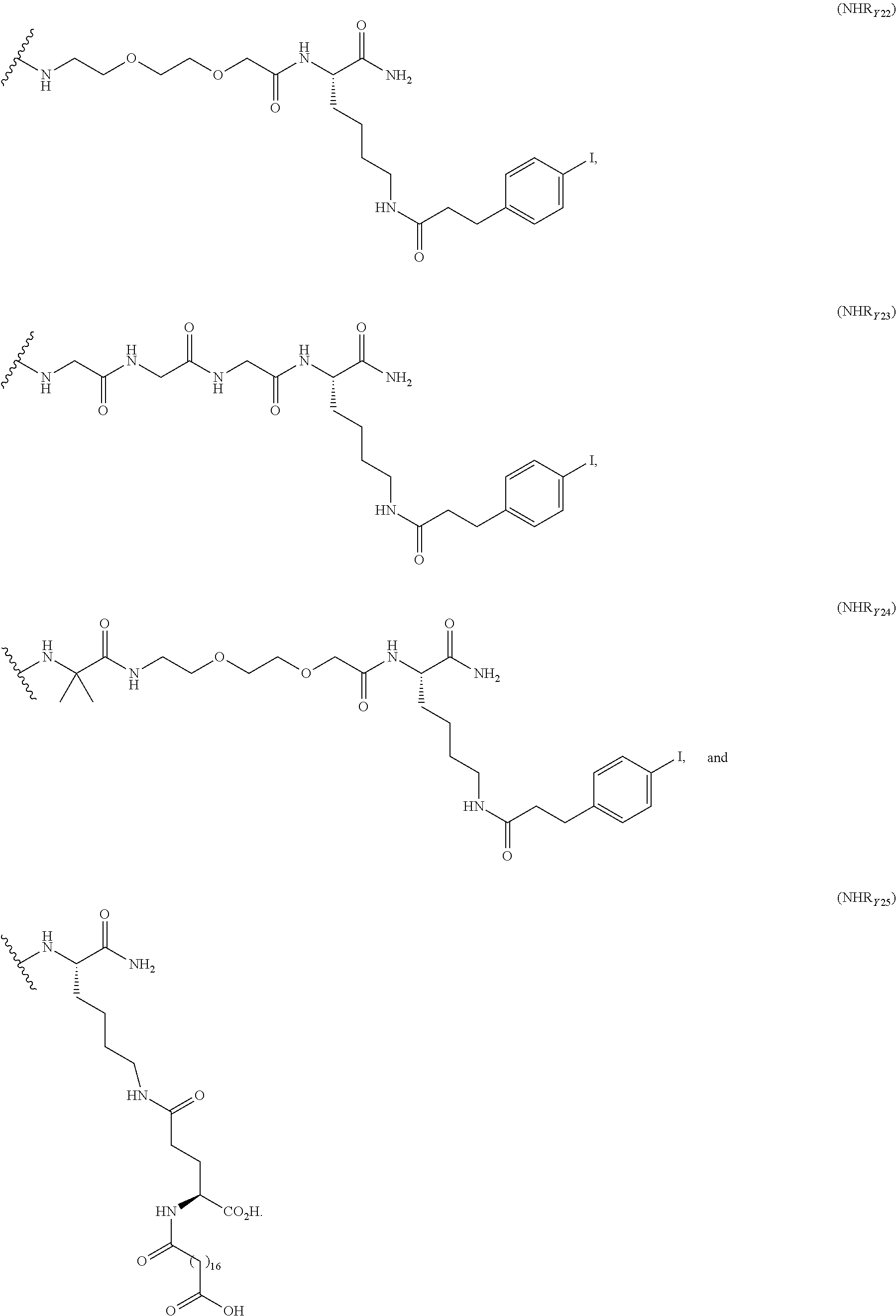

86. The polypeptide of claim 1 selected from the polypeptides recited in Table 4, Table 5 or Table 6.

87.-88. (canceled)

89. The polypeptide of claim 1 represented by:

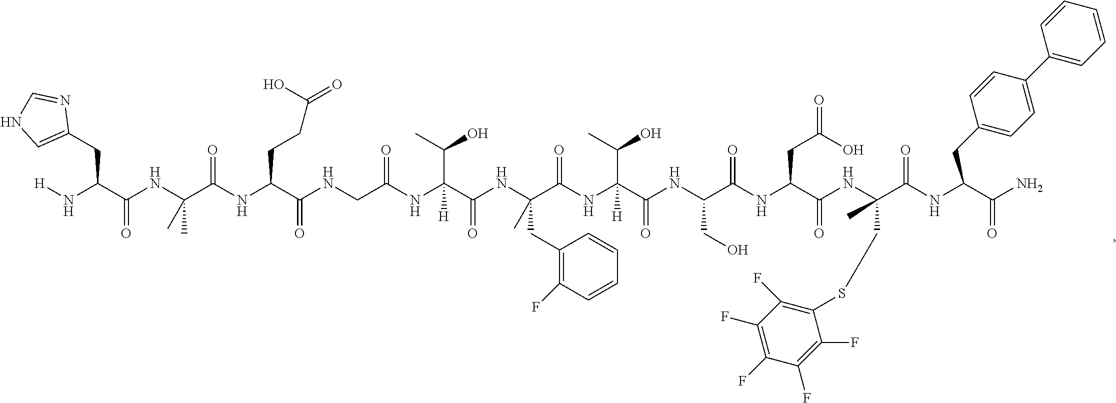

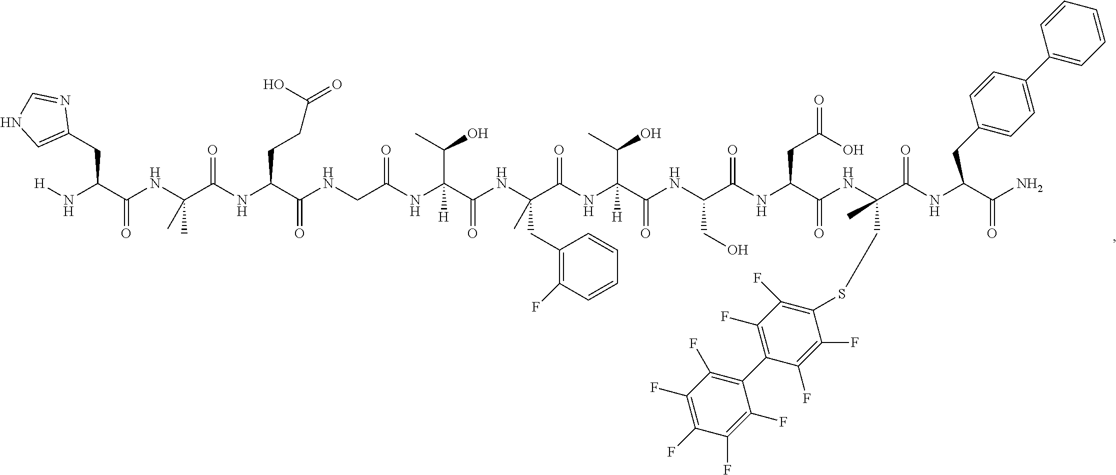

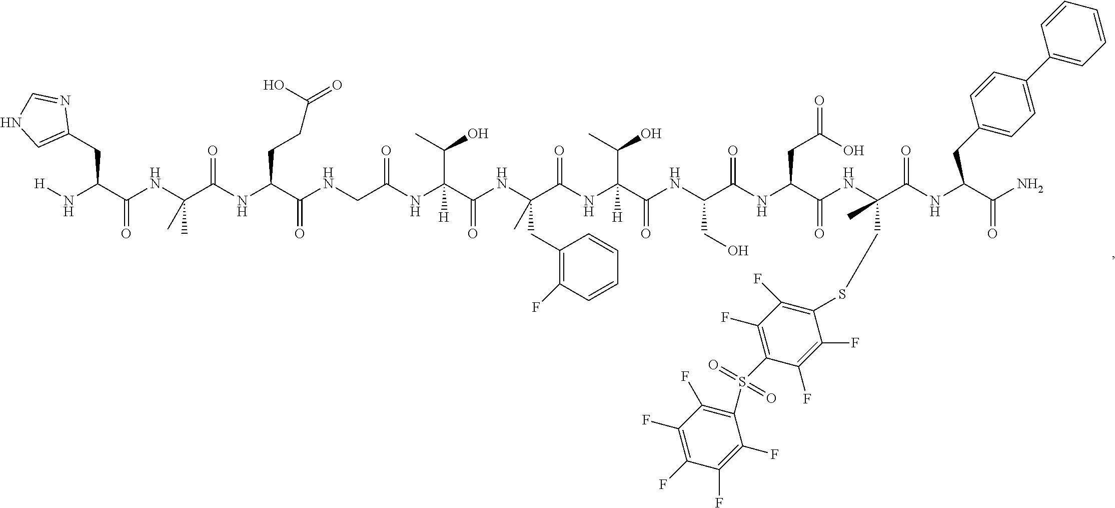

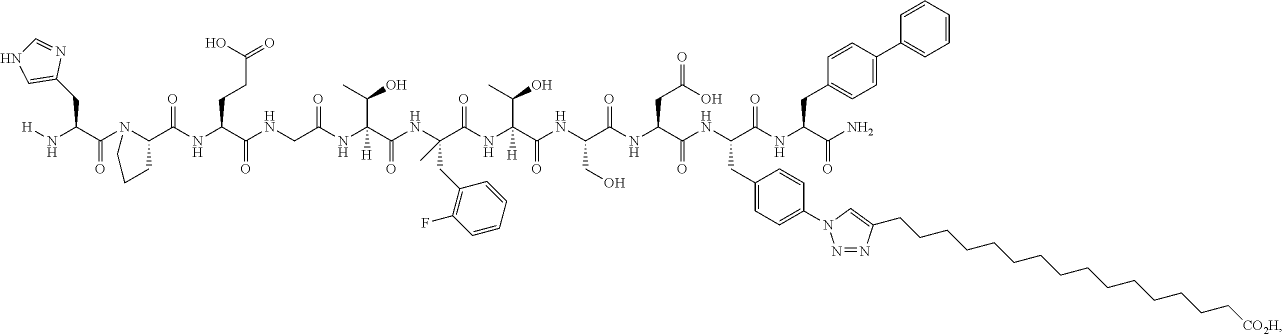 [SMILES: C[C@@H](O)[C@H](NC(=O)CNC(=O)[C@H](CCC(=O)O)NC(=O)[C@@H]1CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-n2cc(CCCCCCCCCCCCCCCC(=O)O)nn2)cc1)C(=O)N[C@@H](Cc1ccc(-c2ccccc2)cc1)C(N)=O)[C@@H](C)O]

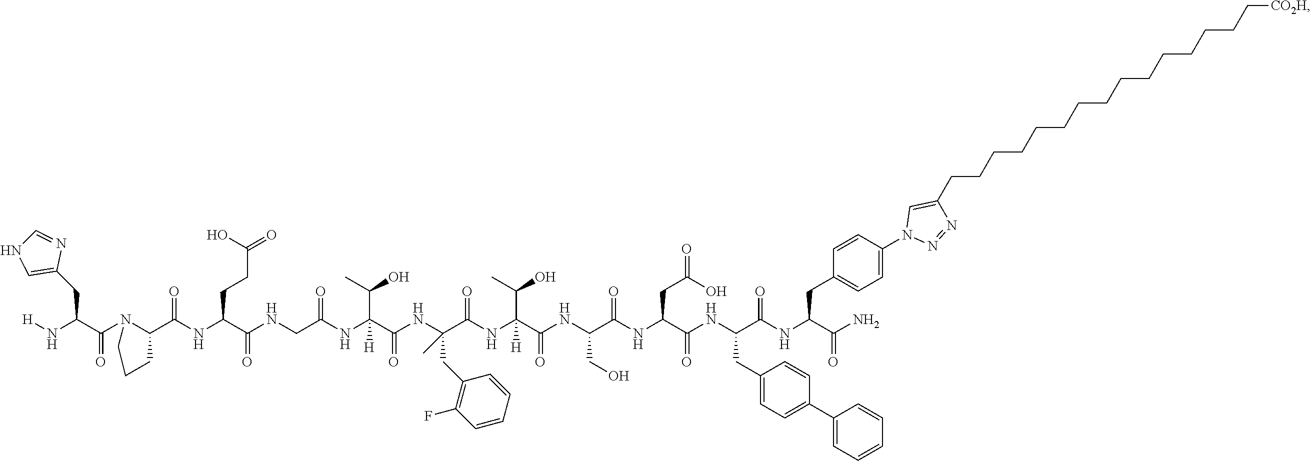 [SMILES: C[C@@H](O)[C@H](NC(=O)CNC(=O)[C@H](CCC(=O)O)NC(=O)[C@@H]1CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-c2ccccc2)cc1)C(=O)N[C@@H](Cc1ccc(-n2cc(CCCCCCCCCCCCCCCC(=O)O)nn2)cc1)C(N)=O)[C@@H](C)O]

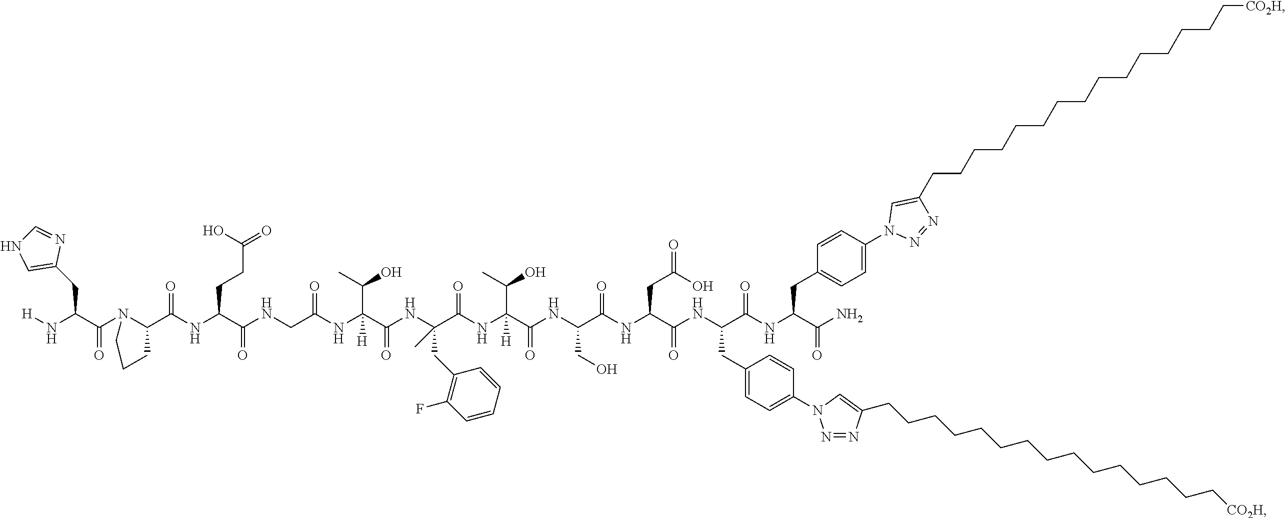 [SMILES: C[C@@H](O)[C@H](NC(=O)CNC(=O)[C@H](CCC(=O)O)NC(=O)[C@@H]1CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-n2cc(CCCCCCCCCCCCCCCC(=O)O)nn2)cc1)C(=O)N[C@@H](Cc1ccc(-n2cc(CCCCCCCCCCCCCCCC(=O)O)nn2)cc1)C(N)=O)[C@@H](C)O]

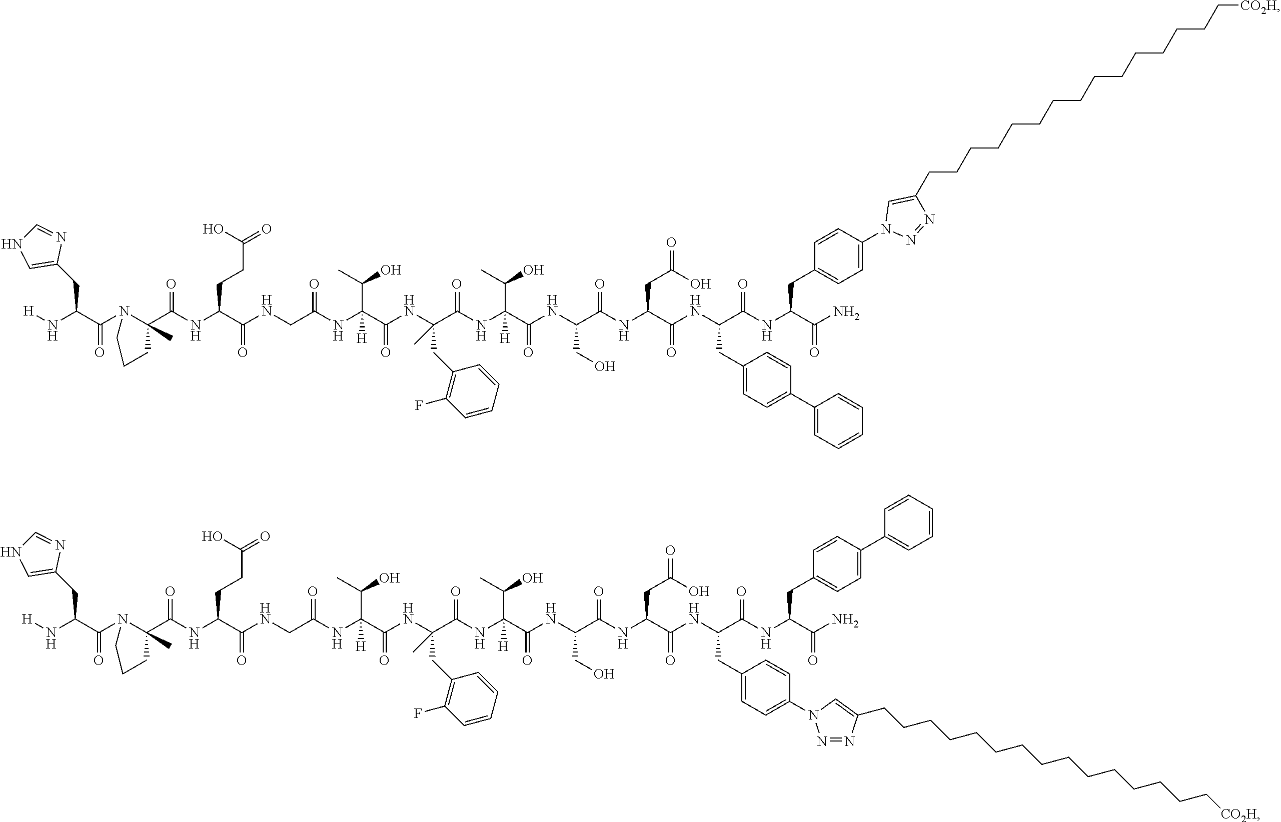

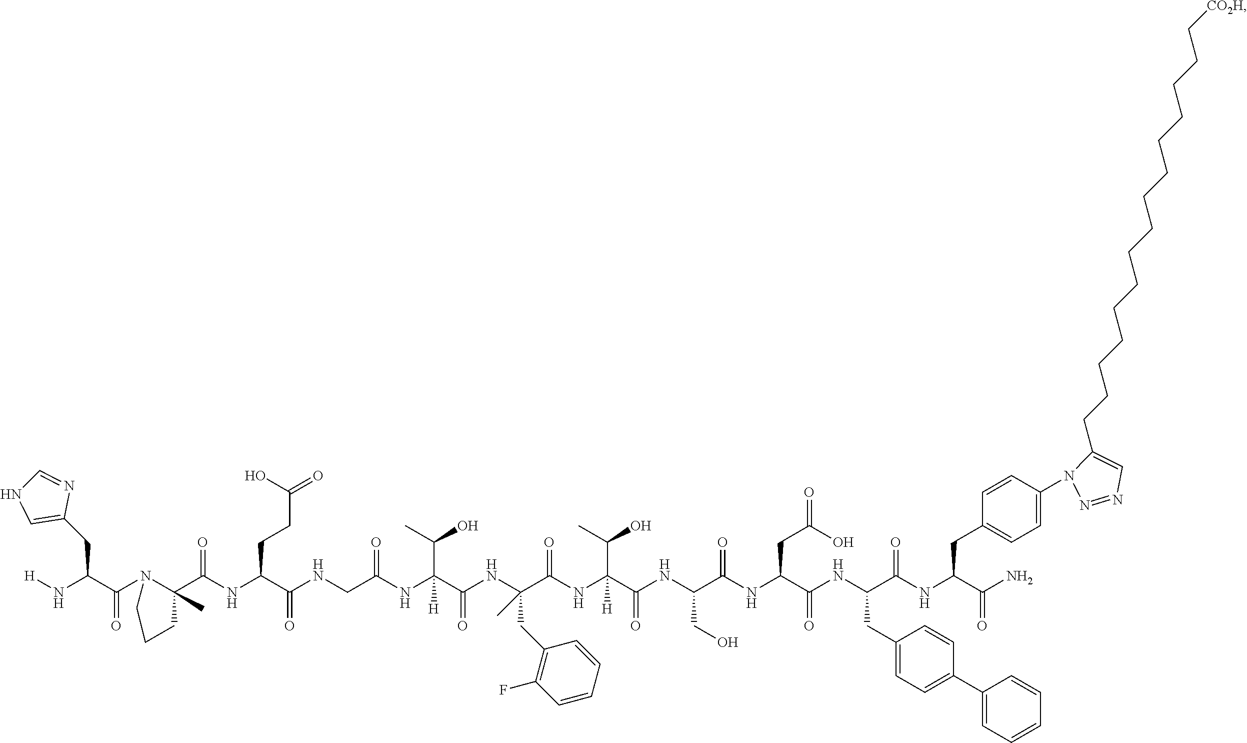 [SMILES: C[C@@H](O)[C@H](NC(=O)CNC(=O)[C@H](CCC(=O)O)NC(=O)[C@]1(C)CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-c2ccccc2)cc1)C(=O)N[C@@H](Cc1ccc(-n2nncc2CCCCCCCCCCCCCCCC(=O)O)cc1)C(N)=O)[C@@H](C)O]

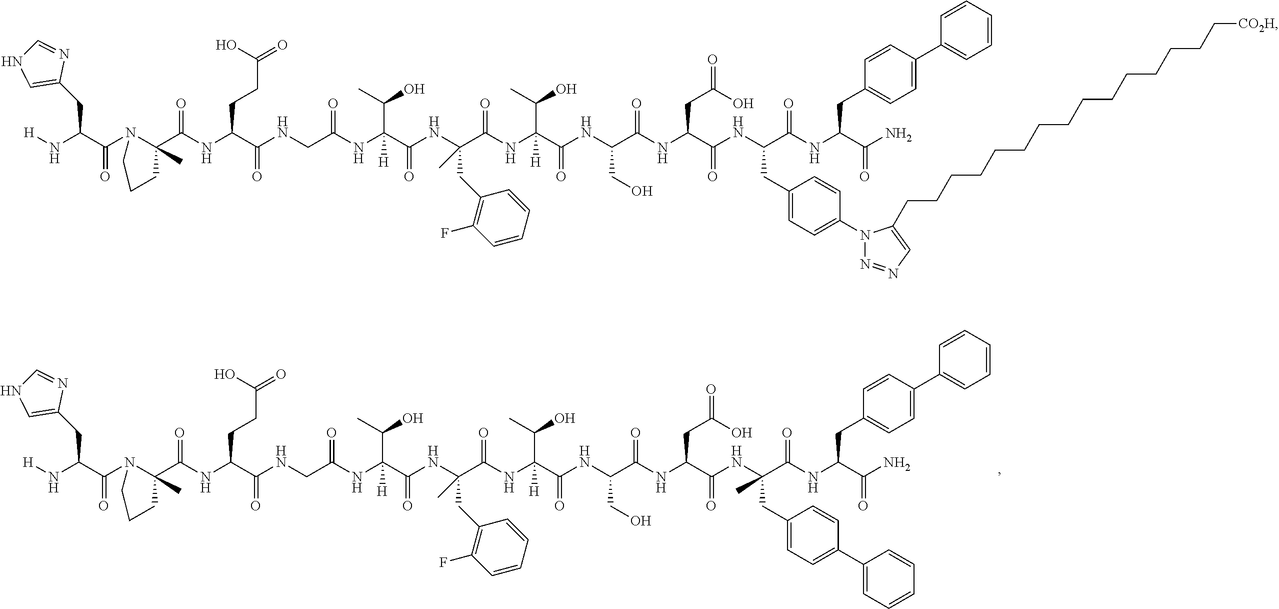

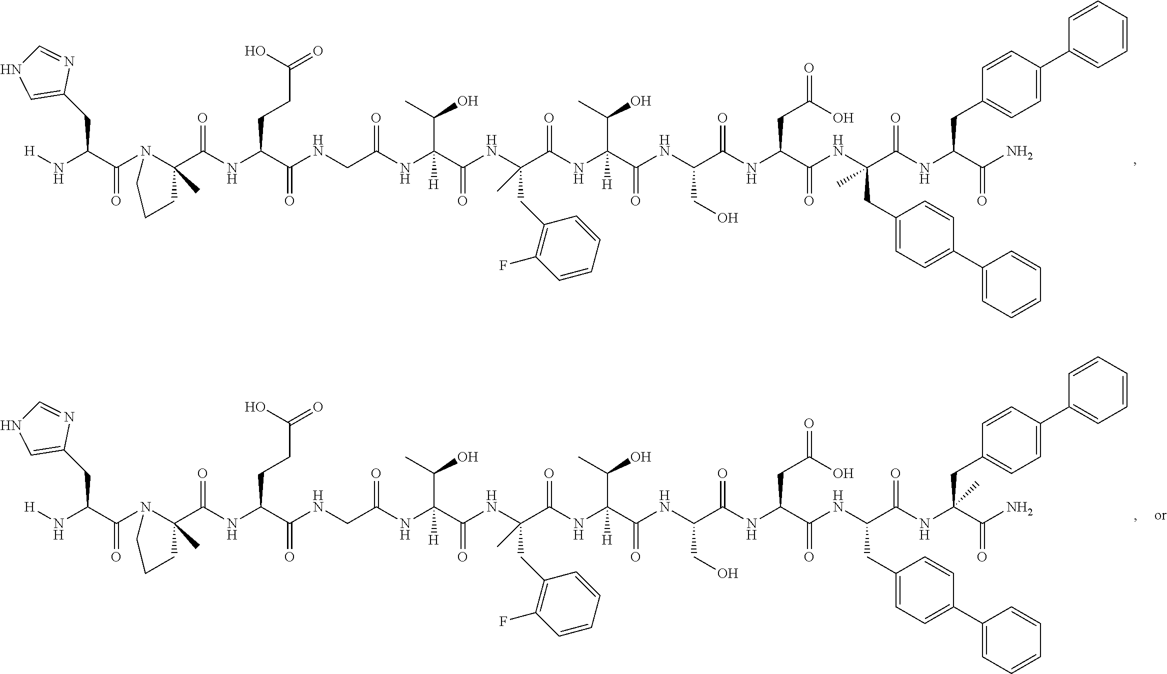

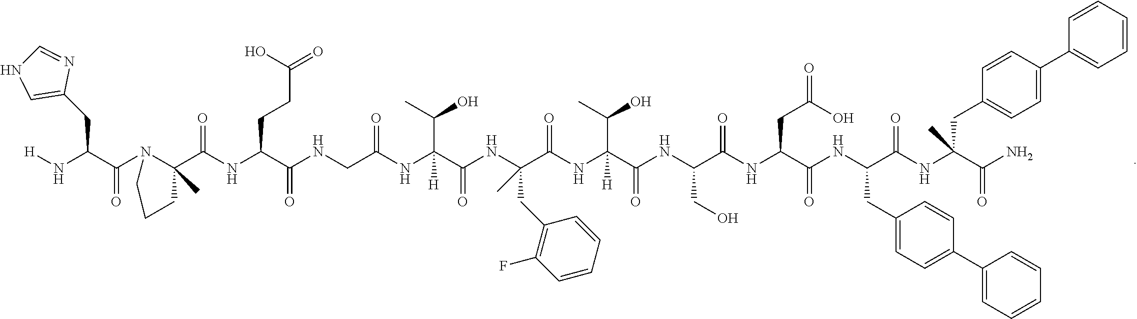 [SMILES: C[C@@H](O)[C@H](NC(=O)CNC(=O)[C@H](CCC(=O)O)NC(=O)[C@]1(C)CCCN1C(=O)[C@@H](N)Cc1c[nH]cn1)C(=O)N[C@@](C)(Cc1ccccc1F)C(=O)N[C@H](C(=O)N[C@@H](CO)C(=O)N[C@@H](CC(=O)O)C(=O)N[C@@H](Cc1ccc(-c2ccccc2)cc1)C(=O)N[C@](C)(Cc1ccc(-c2ccccc2)cc1)C(N)=O)[C@@H](C)O]

90. The polypeptide of claim 1 represented by: 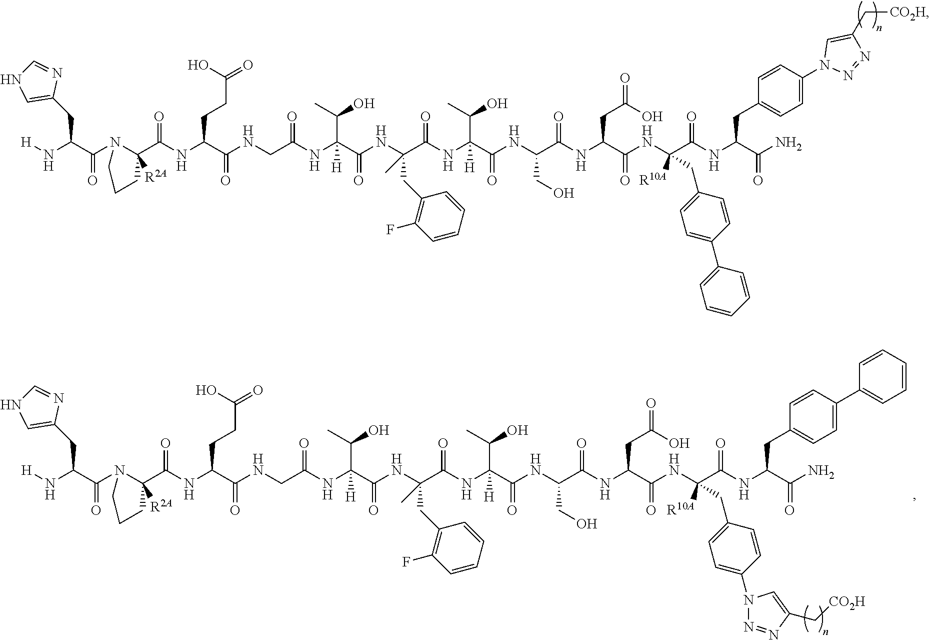 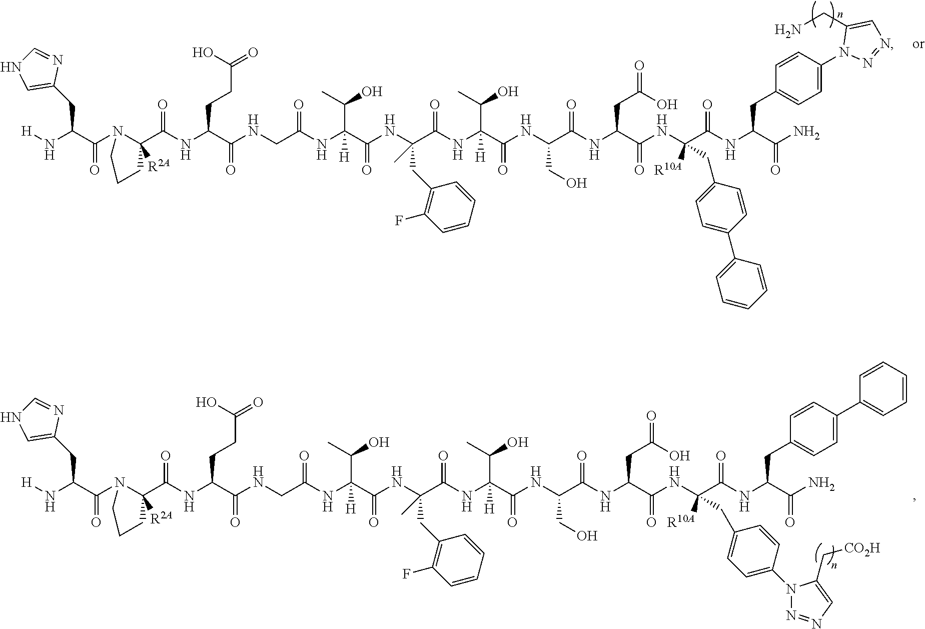
wherein
each n is independently 10 to 20 (e.g., 15);
each R^{2A} is independently —H or alkyl; and.
each R^{10A} is independently —H or alkyl.

91. The polypeptide of claim 1 represented by: 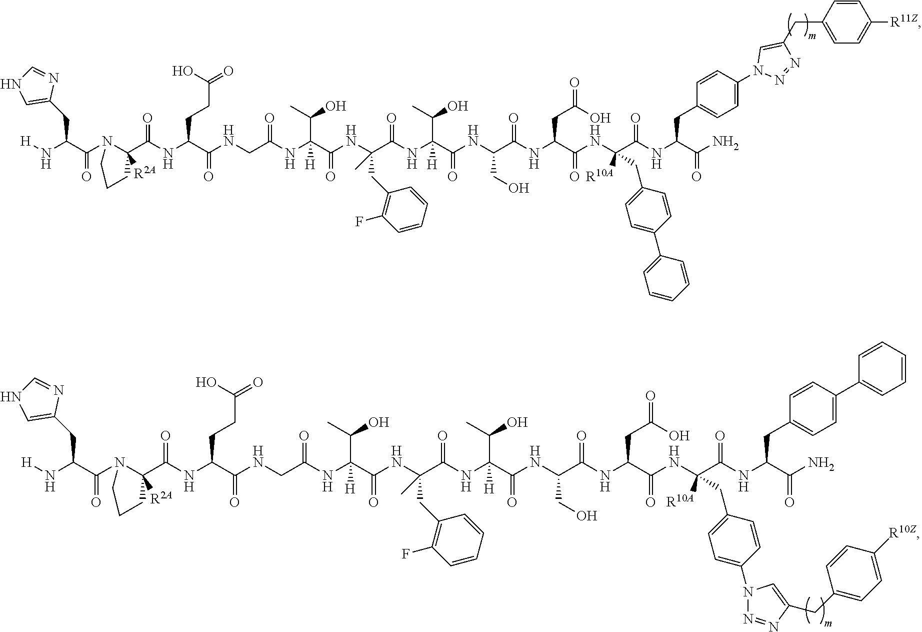 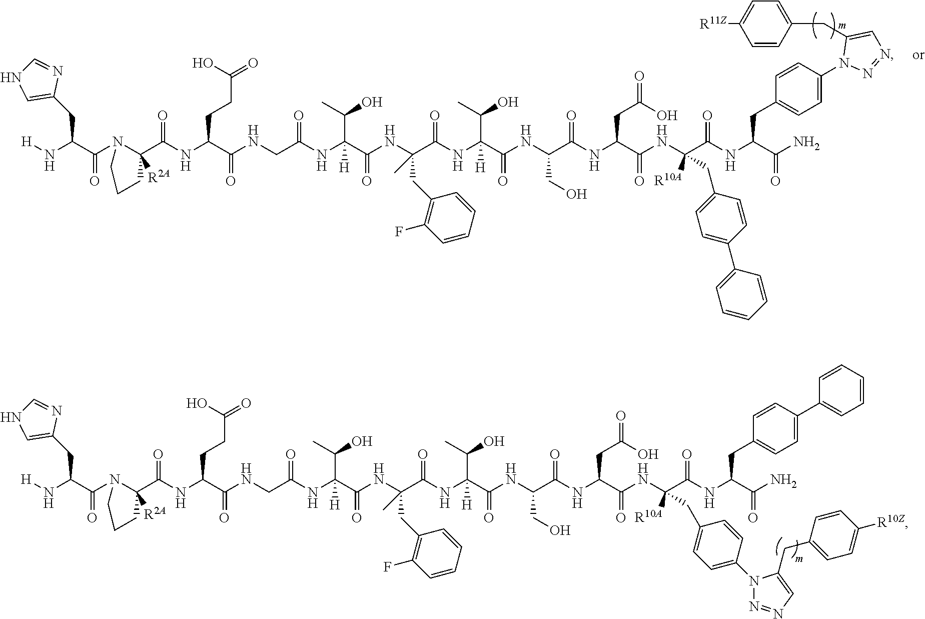
wherein
each m is independently 1 to 10;
each R^{2A} is independently —H or alkyl;
each R^{10A} is independently —H or alkyl;
each R^{10Z} is independently halo (e.g., I); and
each R^{11Z} is independently halo (e.g., I).

92.-94. (canceled)

95. A pharmaceutical composition, comprising a polypeptide of claim 1; and a pharmaceutical acceptable excipient.

96. (canceled)

97. A method of treating or preventing diabetes, obesity, or a neurodegenerative disease or disorder, comprising administering to a subject in need thereof an effective amount of a polypeptide of claim 1,
wherein the neurodegenerative disease or disorder is at least partially mediated by glucagon-like peptide 1 (GLP-1).

98. The method of claim 97, wherein the diabetes is type-II diabetes; and the neurodegenerative disease or disorder is selected from Alzheimer's disease, Parkinson's disease, multiple sclerosis, amyotrophic lateral sclerosis, Huntington's disease, and prion diseases.

99.-103. (canceled)
